(19) United States
(12) Patent Application Publication
(10) Pub. No.: US 2025/0346576 A1
(43) Pub. Date: Nov. 13, 2025

(54) KCNT1 INHIBITORS COMPRISING A PYRAZOLE CORE AND METHODS OF USE

(71) Applicant: PRAXIS PRECISION MEDICINES, INC., Boston, MA (US)

(72) Inventors: Andrew Mark GRIFFIN, L’Île Bizard (CA); Ricardo LIRA, East Lyme, CT (US)

(73) Assignee: PRAXIS PRECISION MEDICINES, INC., Boston, MA (US)

(21) Appl. No.: 18/859,830

(22) PCT Filed: Apr. 24, 2023
(86) PCT No.: PCT/US2023/019653

§ 371 Date: Oct. 24, 2024

Related U.S. Application Data
(60) Provisional application No. 63/334,322, filed on Apr. 25, 2022.
(60) Provisional application No. 63/386,013, filed on Dec. 5, 2022.

(51) Int. Cl. C07D 401/14; A61K 31/4155; A61K 31/4439; A61K 31/454; A61K 31/506; C07D 231/14; C07D 401/04; C07D 403/14

(52) U.S. Cl. CPC C07D 401/14; A61K 31/4155; A61K 31/4439; A61K 31/454; A61K 31/506; C07D 231/14; C07D 401/04; C07D 403/14

(57) ABSTRACT

Disclosed herein are compounds comprising a pyrazole core and pharmaceutically acceptable salts thereof, and compositions useful for preventing and/or treating a neurological disorder, a disorder associated with excessive neuronal excitability, or disorder associated with a gain-of-function mutation in a gene (e.g., KCNT1). Methods of treating a neurological disorder, a disorder associated with excessive neuronal excitability, or disorder associated with a gain-of-function mutation in a gene such as KCNT1 are also provided herein.

### CROSS-REFERENCE TO RELATED APPLICATIONS

This application claims priority to U.S. Provisional Patent Application No. 63/334,322, filed Apr. 25, 2022, and U.S. Provisional Patent Application No. 63/386,013, filed Dec. 5, 2022, the contents of which are incorporated herein by reference in their entireties.

### FIELD OF THE DISCLOSURE

The present disclosure is generally directed to KCNT1 inhibitors comprising a pyrazole core, as well as pharmaceutical compositions and methods of treatment involving the use of such compounds.

### BACKGROUND OF THE DISCLOSURE

Potassium sodium-activated channel subfamily T member 1 (“KCNT1”) is one of the genes in a family of genes responsible for providing the instructions to make potassium channels. KCNT1 encodes sodium-activated potassium channels known as Slack (Sequence like a calcium-activated K⁺ channel). These channels are found in neurons throughout the brain and can mediate a sodium-activated potassium current I_{KNa}. This delayed outward current can regulate neuronal excitability and the rate of adaptation in response to maintained stimulation. Abnormal Slack activity has been associated with development of early onset epilepsies and intellectual impairment. Accordingly, pharmaceutical compounds that selectively regulate sodium-activated potassium channels, e.g., abnormal KCNT1 or abnormal I_{KNa}, are useful in treating a neurological disease or disorder or a disease or condition related to excessive neuronal excitability and/or KCNT1 gain-of-function mutations.

### SUMMARY OF THE DISCLOSURE

Described herein are compounds and compositions useful for preventing and/or treating a disease, disorder, or condition, e.g., a neurological disorder, a disorder associated with excessive neuronal excitability, or disorder associated with a gain-of-function mutation in a gene, for example, KCNT1.

In some aspects, provided is a compound of Formula (I) having a pyrazole core:

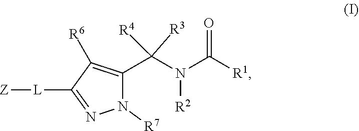

or a pharmaceutically acceptable salt thereof, wherein:
R¹ is chosen from a 5- or 6-membered heteroaryl or aryl, wherein the heteroaryl or aryl optionally comprises at least one substituent independently chosen from an alkyl, a haloalkyl, a carbocyclyl, or —CN;
R² is —H;
R³ is chosen from —H or an alkyl;
R⁴ is chosen from —H or an alkyl,
or R³ and R⁴ are taken together with the carbon atom to which they are attached to form an optionally substituted 3- to 6-membered carbocyclyl or heterocyclyl;
Z is chosen from

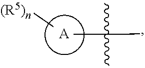

a haloalkyl, or an alkoxy;
ring A is chosen from a 5- or 6-membered heteroaryl, an aryl, a heterocyclyl or a carbocyclyl;
R⁵ is independently chosen from an alkyl, a carbocyclyl, an alkoxy, —C(O)NH₂, —CN, or a halogen, wherein the alkyl, carbocyclyl, or alkoxy optionally comprises at least one halogen substituent, or wherein the alkyl optionally comprises at least one —OH substituent;
n is 0, 1, 2, 3, or 4;
L is absent or is chosen from —NR^{a}—, —CH₂—, or —O—,
R^{a} is chosen from —H or an alkyl,
R⁶ is chosen from —H or an alkyl; and
R⁷ is an alkyl.

In certain embodiments, disclosed herein is a compound of Formula (II) having a pyrazole core:

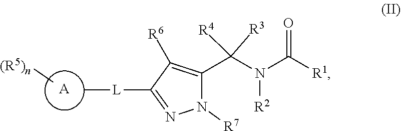

or a pharmaceutically acceptable salt thereof, wherein
R¹ is chosen from a pyrazolyl or a phenyl, wherein the pyrazolyl or phenyl optionally comprises at least one substituent independently chosen from a C₁₋₄ alkyl, a C₁₋₄ haloalkyl, or a C₃₋₅ carbocyclyl;
R² is —H;
R³ is chosen from —H or a C₁₋₄ alkyl;
R⁴ is chosen from —H or a C₁₋₄ alkyl,
or R³ and R⁴ are taken together with the carbon atom to which they are attached to form an optionally substituted 3-5 membered carbocyclyl or heterocyclyl;
ring A is chosen from a pyridyl, a phenyl, a pyrimidinyl, a piperidinyl, or a cyclopentyl,
R⁵ is chosen from a C₁₋₄ alkyl, a C₃₋₅ carbocyclyl, a C₁₋₄ alkoxy, —C(O)NH₂, —CN, or a halogen, wherein the alkyl, carbocyclyl or alkoxy optionally comprises at least one halogen substituent, or wherein the alkyl optionally comprises at least one —OH substituent;
n is 0, 1, 2, 3, or 4;
L is absent or is chosen from —NR^{a}—, —CH₂—, or —O—,
R^{a} is chosen from —H or a C₁₋₄ alkyl,
R⁶ is chosen from —H or a C₁₋₄ alkyl; and
R⁷ is a C₁₋₄ alkyl.

In certain embodiments, R¹ is a pyrazolyl comprising at least one substituent chosen from —CH₃, —CF₃, —C(CH₃)₃, —CHF₂, —CH(CH₃)₂, or a cyclopropyl, and in certain embodiments, R¹ is a phenyl. In certain embodiments, R⁴ is chosen from —H or —CH₃, and in certain embodiments, R³ and R⁴ are taken together with the carbon atom to which they are attached to form an optionally substituted cyclopropyl, cyclobutyl, or oxetanyl. In certain aspects, ring A is a pyridyl, and in certain aspects, R⁵ at each occurrence is independently chosen from —CH₃, —CH₂CH₃, —CF₃, —OCH₃, —OCH₂CH₃, —OCH(CH₃)₂, —CH₂OH, —CN, —C(O)NH₂, or a cyclopropyl. In certain embodiments of the disclosure, n is 0, 1, or 2, and in certain embodiments, L is absent. In certain embodiments, R⁶ is chosen from —H or —CH₃, and in certain embodiments, R⁷ is chosen from —CH₃ or —CH₂CH₃.

In various aspects of the disclosure, the compound of Formula (I) is chosen from a compound of Formula (II-A), (II-B), or (II-C):

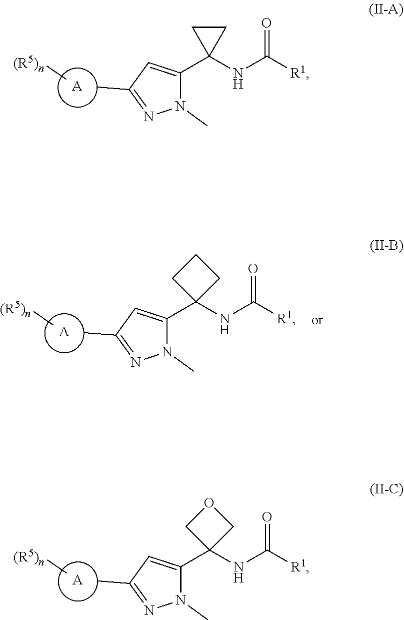

or a pharmaceutically acceptable salt thereof.

In certain embodiments, the compound of Formula (I) is chosen from a compound of Formula (III-A), (III-B), or (III-C):

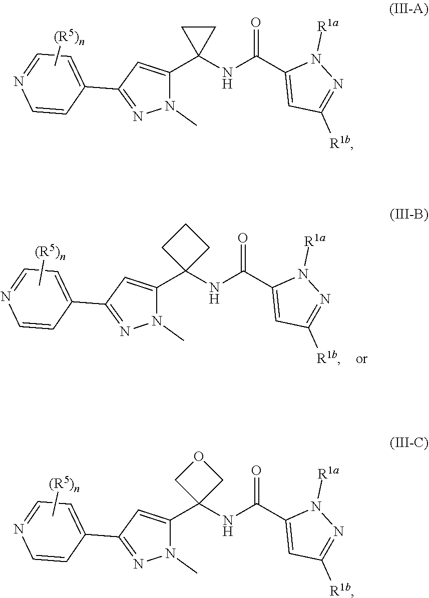

or a pharmaceutically acceptable salt thereof, wherein:
R^{1a} is chosen from —CH₃, —C(CH₃)₃, —CHF₂, —CH(CH₃)₂, or a cyclopropyl;
R^{1b} is chosen from —CH₃, —CF₃, —C(CH₃)₃, —CHF₂, —CH(CH₃)₂, or a cyclopropyl; and
R⁵ at each occurrence is independently chosen from a C₁₋₄ alkyl, a C₁₋₄ haloalkyl, a C₁₋₄ alkoxy, or a C₃₋₅ carbocyclyl.

In certain embodiments, the compound of Formula (I) is chosen from a compound of Formula (III-A-i), (III-B-i), or (III-C-i):

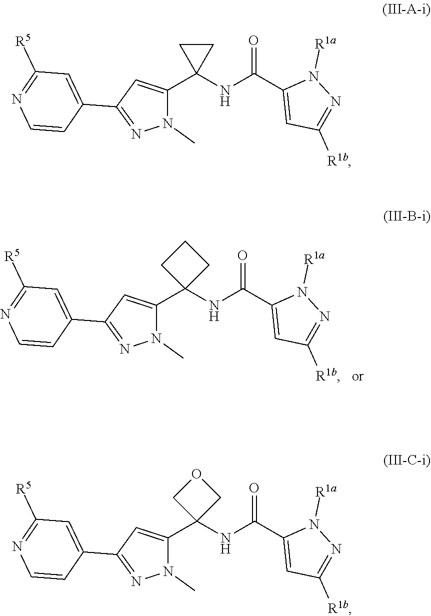

or a pharmaceutically acceptable salt thereof, wherein:
R^{1a} is chosen from —CH₃, —C(CH₃)₃, —CHF₂, —CH(CH₃)₂, or a cyclopropyl;
R^{1b} is chosen from —CH₃, —CF₃, —C(CH₃)₃, —CHF₂, —CH(CH₃)₂, or a cyclopropyl; and
R⁵ is chosen from —CF₃, —CH₃, —CH₂CH₃, —OCH₃, —OCH₂CH₃, or a cyclopropyl.

In certain embodiments, disclosed herein is a compound of Formula (I) chosen from a compound of Formula (III-D-i) or (III-F-i):

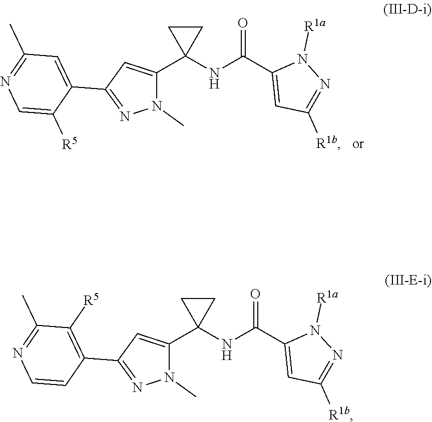

or a pharmaceutically acceptable salt thereof, wherein:
R^{1a} is chosen from —CH₃, —C(CH₃)₃, —CHF₂, —CH(CH₃)₂, or a cyclopropyl;
R^{1b} is chosen from —CH₃, —CF₃, —C(CH₃)₃, —CHF₂, —CH(CH₃)₂, or a cyclopropyl; and
R⁵ is chosen from —F or —CN.

In certain embodiments, the compound of Formula (I) is chosen from:

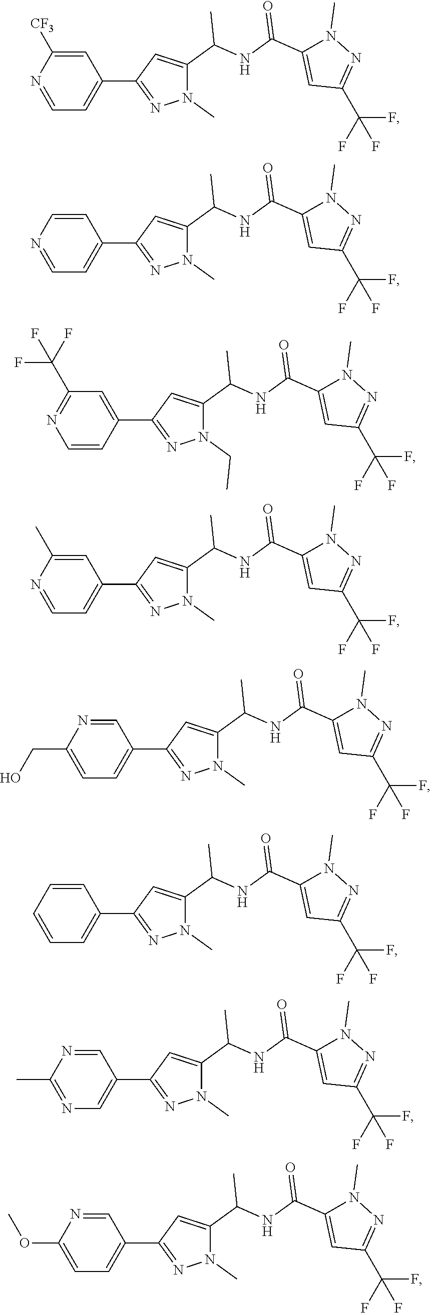

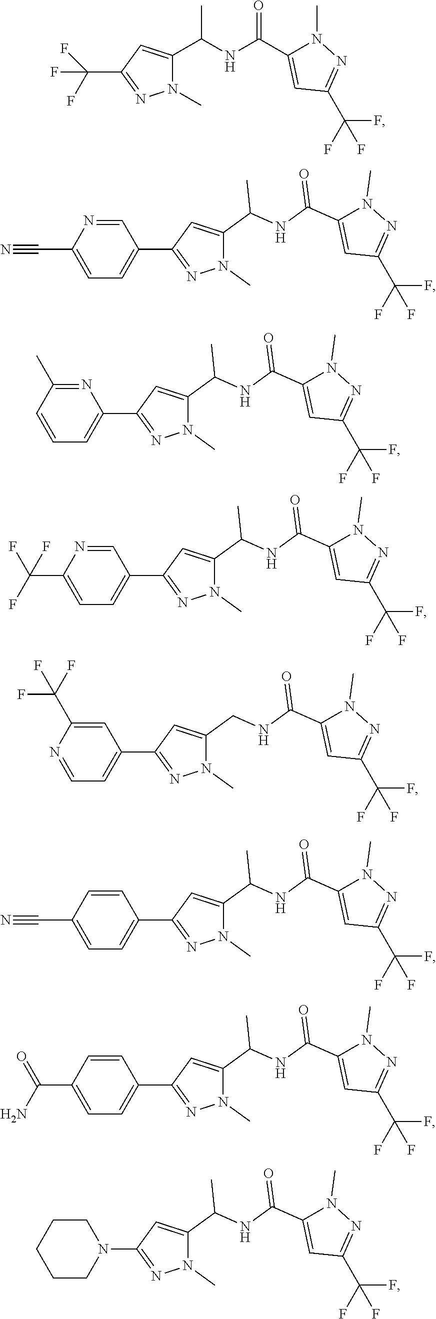

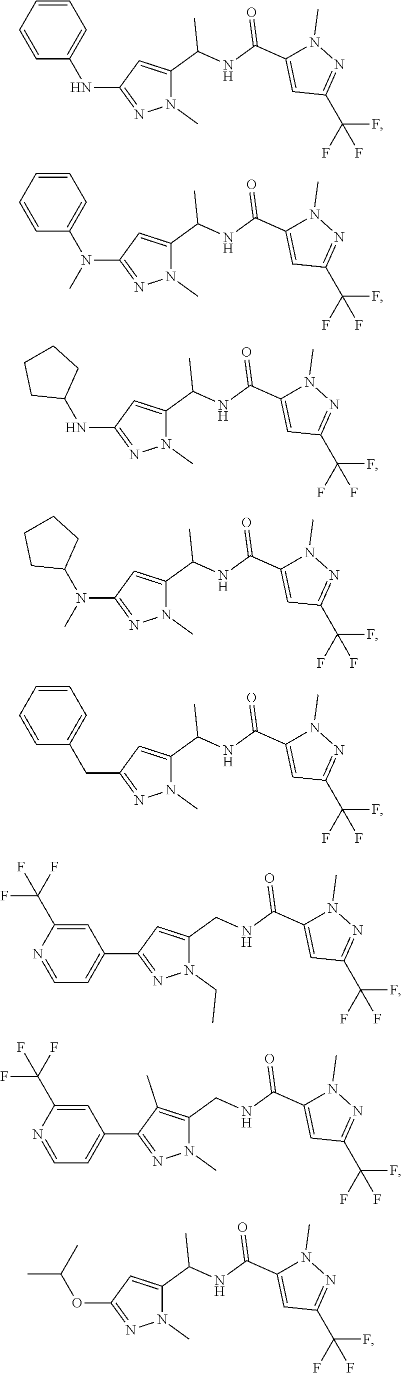

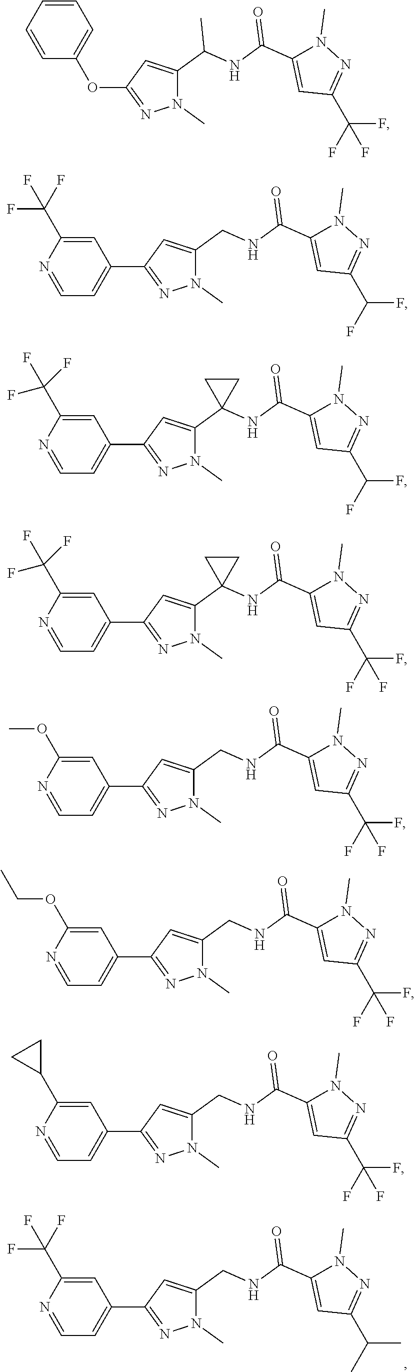

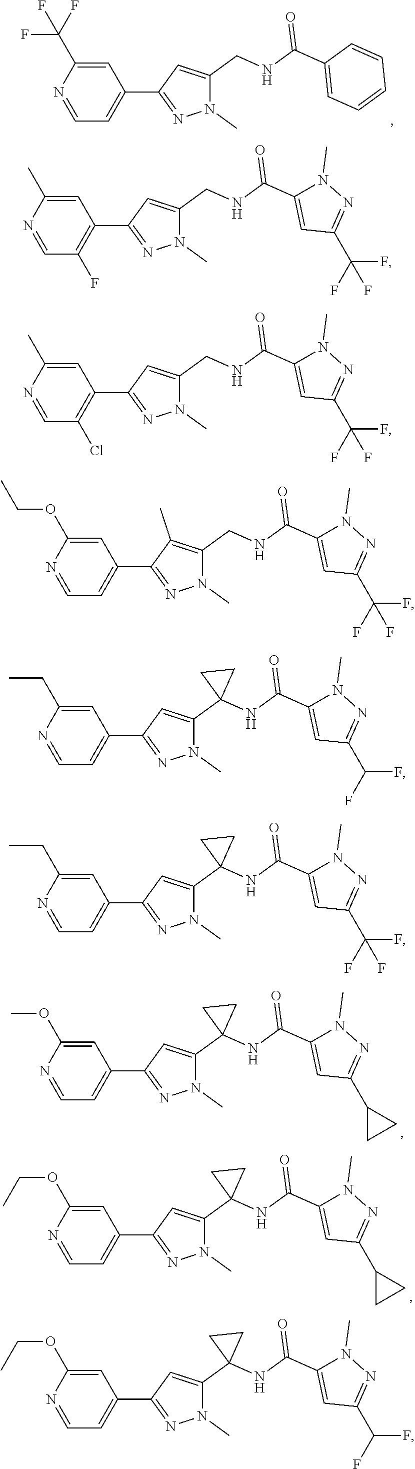

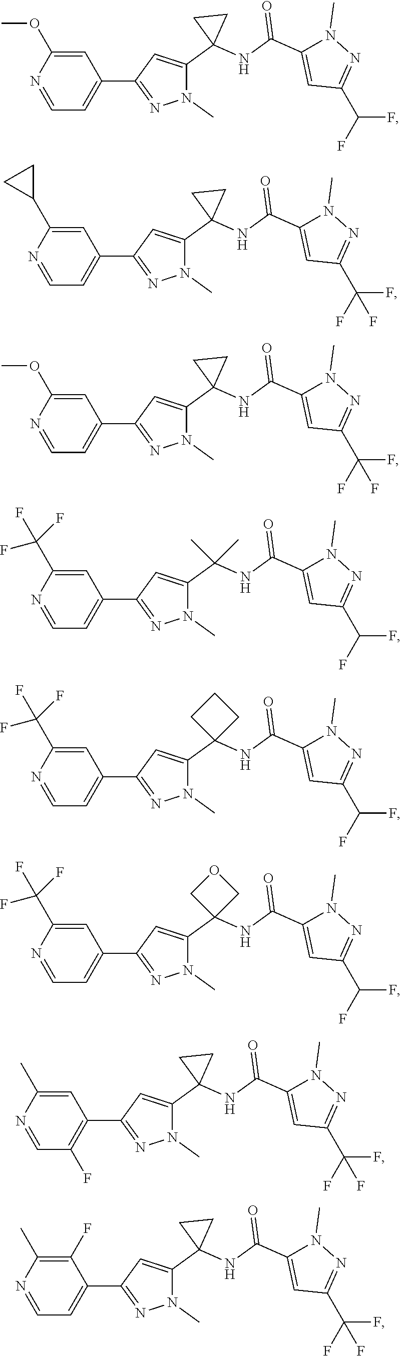

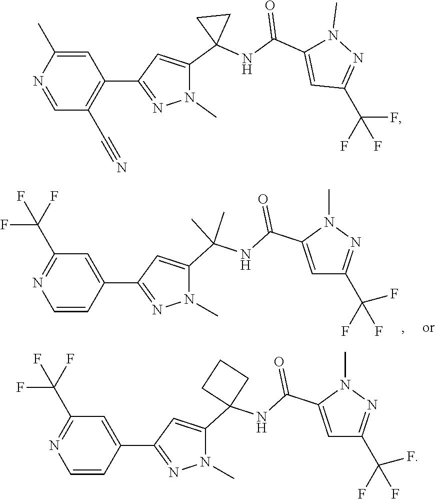

In other aspects, provided is a method of treating a neurological disorder, a disorder associated with excessive neuronal excitability, or a disorder associated with a gain-of-function mutation of a gene, by administering to a subject in need thereof an effective amount of any of the compounds described herein or a pharmaceutically acceptable salt thereof, or pharmaceutical compositions described herein comprising such compounds or a pharmaceutically acceptable salt thereof.

In some embodiments, the method provided involves treating a disorder associated with a gain-of-function mutation of KCNT1.

In some variations, the neurological disorder, the disorder associated with excessive neuronal excitability, or the disorder associated with a gain-of-function mutation of a gene (e.g., KCNT1) is epilepsy, an epilepsy syndrome, or an encephalopathy.

In some variations, the neurological disorder, the disorder associated with excessive neuronal excitability, or the disorder associated with a gain-of-function mutation of a gene (e.g., KCNT1) is a genetic or pediatric epilepsy or a genetic or pediatric epilepsy syndrome.

In some variations, the neurological disorder, the disorder associated with excessive neuronal excitability, or the disorder associated with a gain-of-function mutation of a gene (e.g., KCNT1) is a cardiac dysfunction.

In some variations, the neurological disorder, the disorder associated with excessive neuronal excitability, or the disorder associated with a gain-of-function mutation of a gene (e.g., KCNT1) is selected from the group consisting of epilepsy and other encephalopathies (e.g., malignant migrating focal seizures of infancy (MMFSI) or epilepsy of infancy with migrating focal seizures (EIMFS), autosomal dominant nocturnal frontal lobe epilepsy (ADNFLE), West syndrome, infantile spasms, epileptic encephalopathy, focal epilepsy, Ohtahara syndrome, developmental and epileptic encephalopathy, Lennox-Gastaut syndrome, seizures (e.g., Generalized tonic clonic seizures, Asymmetric Tonic Seizures), leukodystrophy, leukoencephalopathy, intellectual disability, Multifocal Epilepsy, Drug resistant epilepsy, Temporal lobe epilepsy, or cerebellar ataxia.

In some variations, the neurological disorder, the disorder associated with excessive neuronal excitability, or the disorder associated with a gain-of-function mutation of a gene (e.g., KCNT1) is chosen from cardiac arrhythmia, Brugada syndrome, or myocardial infarction.

In some variations, the neurological disorder, the disorder associated with excessive neuronal excitability, or the disorder associated with a gain-of-function mutation of a gene (e.g., KCNT1) is selected from pain and related conditions (e.g., neuropathic pain, acute/chronic pain, migraine).

In some variations, the neurological disorder, the disorder associated with excessive neuronal excitability, or the disorder associated with a gain-of-function mutation of a gene (e.g., KCNT1) is a muscle disorder (e.g., myotonia, neuromyotonia, cramp muscle spasms, spasticity).

In some variations, the neurological disorder, the disorder associated with excessive neuronal excitability, or the disorder associated with a gain-of-function mutation of a gene (e.g., KCNT1) is selected from itch and pruritis, ataxia, or cerebellar ataxias.

In some variations, the neurological disorder, the disorder associated with excessive neuronal excitability, or the disorder associated with a gain-of-function mutation of a gene (e.g., KCNT1) is a psychiatric disorder (e.g., major depression, anxiety, bipolar disorder, schizophrenia).

In other variations, the neurological disorder, the disorder associated with excessive neuronal excitability, or the disorder associated with a gain-of-function mutation in a gene (e.g., KCNT1) is chosen from a learning disorder, Fragile X, neuronal plasticity, or an autism spectrum disorder.

In yet other variations, the neurological disorder, the disorder associated with excessive neuronal excitability, or the disorder associated with a gain-of-function mutation of a gene (e.g., KCNT1) is chosen from epileptic encephalopathy with SCN1A, SCN2A, and/or SCN8A mutations, early infantile epileptic encephalopathy, Dravet syndrome, Dravet syndrome with SCN1A mutation, generalized epilepsy with febrile seizures, intractable childhood epilepsy with generalized tonic-clonic seizures, infantile spasms, benign familial neonatal-infantile seizures, SCN2A epileptic encephalopathy, focal epilepsy with SCN3A mutation, cryptogenic pediatric partial epilepsy with SCN3A mutation, SCN8A epileptic encephalopathy, Rasmussen encephalitis, malignant migrating partial seizures of infancy, autosomal dominant nocturnal frontal lobe epilepsy, KCNQ2 epileptic encephalopathy, or KCNT1 epileptic encephalopathy.

Other objects and advantages will become apparent to those skilled in the art from consideration of the ensuing description.

### DESCRIPTION OF THE DISCLOSURE

Provided herein, in certain aspects, are compounds and compositions useful for preventing and/or treating a disease, disorder, or condition described herein, e.g., a neurological disorder, a disorder associated with excessive neuronal excitability, or a disorder associated with gain-of-function mutations in a gene (e.g., KCNT1). Exemplary diseases, disorders, or conditions include epilepsy and other encephalopathies (e.g., MMFSI or EIMFS, ADNFLE, West syndrome, infantile spasms, epileptic encephalopathy, focal epilepsy, Ohtahara syndrome, developmental and epileptic encephalopathy, Lennox-Gastaut syndrome, seizures, leukodystrophy, leukoencephalopathy, Intellectual disability, Multifocal Epilepsy, Generalized tonic clonic seizures, Drug resistant epilepsy, Temporal lobe epilepsy, cerebellar ataxia, Asymmetric Tonic Seizures); cardiac dysfunctions (e.g., cardiac arrhythmia, Brugada syndrome, myocardial infarction); pain and related conditions (e.g., neuropathic pain, acute/chronic pain, migraine, etc.); muscle disorders (e.g., myotonia, neuromyotonia, cramp muscle spasms, spasticity); itch and pruritis; ataxia and cerebellar ataxias; and psychiatric disorders (e.g., major depression, anxiety, bipolar disorder, schizophrenia).

### I. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For instance, where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure. In some embodiments, two opposing and open-ended ranges are provided for a feature, and in such description it is envisioned that combinations of those two ranges are provided herein. For example, in some embodiments, it is described that a feature is greater than about 10 units, and it is described (such as in another sentence) that the feature is less than about 20 units, and thus, the range of about 10 units to about 20 units is described herein.

The term “about” as used herein refers to the usual error range, for the respective value readily known in this technical field. Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise. For example, “a” or “an” means “at least one” or “one or more.” It is understood that aspects and variations described herein include embodiments “consisting” and/or “consisting essentially of” such aspects and variations.

The terms “disease,” “disorder,” and “condition” are used interchangeably herein.

As used herein, the term “in some embodiments,” “in other embodiments,” or the like, refers to embodiments of all aspects of the disclosure, unless the context clearly indicates otherwise.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described, for example, in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The following terms are intended to have the meanings presented therewith below and are useful in understanding the description and intended scope of the present disclosure. When describing certain aspects of the disclosure, which may include compounds, pharmaceutical compositions containing such compounds, and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein. The articles “a” and “an” may be used herein to refer to one or to more than one (i.e., at least one) of the grammatical objects of the article. By way of example “an analogue” means one analogue or more than one analogue.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Alkyl” refers to a radical of a straight-chain or branched saturated hydrocarbon group, e.g., having 1 to 20 carbon atoms (“C₁₋₂₀ alkyl”). In some embodiments, an alkyl group has 1 to 10 carbon atoms (“C₁₋₁₀ alkyl”). In some embodiments, an alkyl group has 1 to, 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). Examples of C₁₋₆ alkyl groups include methyl, ethyl, propyl, isopropyl, butyl, isobutyl, pentyl, hexyl, and the like.

“Alkenyl” refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds) (“C₂₋₂₀ alkenyl”). In certain embodiments, alkenyl does not contain any triple bonds. In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like.

“Alkoxy” refers to a radical of a straight-chain or branched hydrocarbon group, e.g., having 1 to 20 carbon atoms, having a single bond to oxygen. In some embodiments, an alkoxy has 1-2 carbon atoms, such as —OCH₃ or —OCH₂CH₃.

“Alkynyl” refers to a radical of a straight-chain or branched hydrocarbon group having from 2 to 20 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds) (“C₂₋₂₀ alkynyl”). In certain embodiments, alkynyl does not contain any double bonds. In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like.

“Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particularly aryl groups include phenyl, naphthyl, indenyl, and tetrahydronaphthyl.

“Hetero” when used to describe a compound or a group present on a compound means that one or more carbon atoms in the compound or group have been replaced by a nitrogen, oxygen, or sulfur heteroatom. Hetero may be applied to any of the alkyl groups described above such as alkyl, e.g., heteroalkyl; alkenyl, e.g., heteroalkenyl; alkynyl, e.g., heteroalkynyl; carbocyclyl, e.g., heterocyclyl; aryl, e.g., heteroaryl, and the like having from 1 to 5, and particularly from 1 to 3 heteroatoms.

“Heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur.

“Carbocyclyl,” or “carbocyclic” refers to a radical of a non-aromatic cyclic hydrocarbon group having from 3 to 10 ring carbon atoms (“C₃₋₁₀ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₅ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system.

“Heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spire ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing three heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Cyano” refers to —CN.

“Halo” or “halogen” refers to a fluorine atom (i.e., fluoro or —F), a chlorine atom (i.e., chloro or —Cl), a bromine atom (i.e., bromo or —Br), and an iodine atom (i.e., iodo or —I). In certain embodiments, the halo group is fluoro or chloro.

“Haloalkyl” refers to an alkyl group substituted with one or more halogen atoms.

In general, the term “substituted,” whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position.

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. The general concept of pharmaceutically acceptable salts has been discussed in the art, including, for example, Berge et al., which describes pharmaceutically acceptable salts in detail in J Pharmaceutical Sciences (1977) 66: 1-19. Pharmaceutically acceptable salts of the compounds described herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “modified-release polymer” refers to a polymer that is used in a formulation (e.g., tablets and capsules) to modify the release rate of the drug upon administration to a subject. For example, a modified-release polymer is used to dissolve a drug over time in order to be released slower and steadier into the bloodstream. For example, a modified-release polymer is a controlled-release polymer. For example, a modified-release polymer or a controlled-release polymer is an HPMC polymer. In some embodiments, a modified-release polymer may include hydrophilic matrix polymers (e.g., hypromellose, hydroxyl-propyl methylcellulose (HPMC)), hydrophobic matrix polymers (e.g., ethyl cellulose, ethocel), or polyacrylate polymers (e.g., Eudragit® RL100, Eudragit® RS100).

The term “diluent” as used herein refers to an excipient used to increase weight and improve content uniformity. For example, diluents include cellulose derivatives (e.g., microcrystalline cellulose), starches (e.g., hydrolyzed starches, and partially pregelatinized starches), anhydrous lactose, lactose monohydrate, di-calcium phosphate (DCP), sugar alcohols (e.g., sorbitol, xylitol and mannitol)).

The term “glidant” as used herein refers to an excipient used to promote powder flow by reducing interparticle friction and cohesion. For example, glidants include fumed silica (e.g., colloidal silicon dioxide), talc, and magnesium carbonate.

The term “lubricant” as used herein refers to an excipient used to prevent ingredients from clumping together and from sticking to the tablet punches or capsule filling machine. Lubricants are also used to ensure that tablet formation and ejection can occur with low friction between the solid and die wall. For example, lubricants include magnesium stearate, calcium stearate, stearic acid, talc, silica, and fats (e.g., vegetable stearin).

The term “coating” as used herein refers to an excipient to protect tablet ingredients from deterioration by moisture in the air and make large or unpleasant-tasting tablets easier to swallow.

The embodiments disclosed herein are not intended to be limited in any manner by the above exemplary listing of chemical groups and substituents. Those skilled in the art will recognize that several embodiments are possible within the scope and spirit of the present disclosure. The following description illustrates the disclosure and, of course, should not be construed in any way as limiting the scope of the inventions described herein.

### II. Compounds and Compositions

In one aspect, provided is a compound of Formula (I) having a pyrazole core:

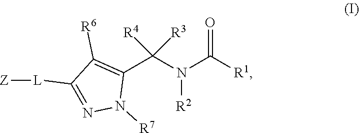

or a stereoisomer thereof, or a pharmaceutically acceptable salt thereof, wherein:
R¹ is chosen from a 5- or 6-membered heteroaryl or an aryl, wherein the heteroaryl or aryl optionally comprises at least one substituent independently chosen from an alkyl, a haloalkyl, a carbocyclyl, or —CN;
R² is —H;
R³ is chosen from —H or an alkyl;
R⁴ is chosen from —H or an alkyl,
or R³ and R⁴ are taken together with the carbon atom to which they are attached to form an optionally substituted 3- to 6-membered carbocyclyl or heterocyclyl;
Z is chosen from

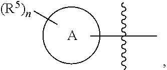

a haloalkyl, or an alkoxy;
ring A is chosen from a 5- or 6-membered heteroaryl, an aryl, a heterocyclyl, or a carbocyclyl;
R⁵ is independently chosen from an alkyl, a carbocyclyl, an alkoxy, —C(O)NH₂, —CN, or a halogen, wherein the alkyl, carbocyclyl or alkoxy optionally comprises at least one halogen substituent, or wherein the alkyl optionally comprises at least one —OH substituent;
n is 0, 1, 2, 3, or 4;
L is absent or is chosen from —NR^{a}—, —CH₂—, or —O—,
R^{a} is chosen from —H or an alkyl,
R⁶ is chosen from —H or an alkyl; and
R⁷ is alkyl.

In some embodiments, provided is a compound of Formula (II) having a pyrazole core:

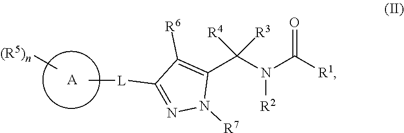

or a stereoisomer thereof, or a pharmaceutically acceptable salt thereof, wherein:
R¹ is chosen from a pyrazolyl or a phenyl, wherein the pyrazolyl or phenyl optionally comprises at least one substituent independently chosen from a C₁₋₄ alkyl, a C₁₋₄ haloalkyl, or a C₃₋₅ carbocyclyl;
R² is —H;
R³ is chosen from —H or an alkyl;
R⁴ is chosen from —H or a C₁₋₄ alkyl,
or R³ and R⁴ are taken together with the carbon atom to which they are attached to form an optionally substituted 3-5 membered carbocyclyl or heterocyclyl;
ring A is chosen from a pyridyl, a phenyl, a pyrimidinyl, a piperidinyl, or a cyclopentyl,
R⁵ is chosen from a C₁₋₄ alkyl, a C₃₋₅ carbocyclyl, a C₁₋₄ alkoxy, —C(O)NH₂, —CN, or a halogen, wherein the alkyl, carbocyclyl, or alkoxy optionally comprises at least one halogen substituent, or wherein the alkyl optionally comprises at least one —OH substituent;
n is 0, 1, 2, 3, or 4;
L is absent or is chosen from —NR^{a}—, —CH₂—, or —O—,
R^{a} is chosen from —H or a C₁₋₄ alkyl,
R⁶ is chosen from —H or a C₁₋₄ alkyl; and
R⁷ is a C₁₋₄ alkyl.

In some variations of all of the foregoing, the compound is an optically active compound. In some variations, the compound is a single enantiomer. In certain variations, the compound is the (R)-enantiomer. In other variations, the compound is the (S)-enantiomer.

In some embodiments, R¹ is a pyrazolyl comprising at least one substituent independently chosen from —CH₃, —CF₃, —C(CH₃)₃, —CHF₂, —CH(CH₃)₂, or a cyclopropyl. In certain variations, R¹ is a phenyl.

In some embodiments, R⁴ is chosen from —H or —CH₃. In other embodiments, R³ and R⁴ are taken together with the carbon atom to which they are attached to form an optionally substituted cyclopropyl, cyclobutyl, or oxetanyl.

In some embodiments, ring A is a pyridyl. In certain embodiments, R⁵ is at each occurrence independently —CH₃, —CH₂CH₃, —CF₃, —OCH₃, —OCH₂CH₃, —OCH(CH₃)₂, —CH₂OH, —CN, —C(O)NH₂, or a cyclopropyl.

In some embodiments, n is 0, 1, or 2. In some embodiments, L is absent.

In some embodiments, R⁶ is chosen from —H or —CH₃. In other embodiments, R⁷ is chosen from —CH₃ or —CH₂CH₃.

In certain aspects, provided is a compound of Formula (II-A), (II-B) or (II-C) having a pyrazole core:

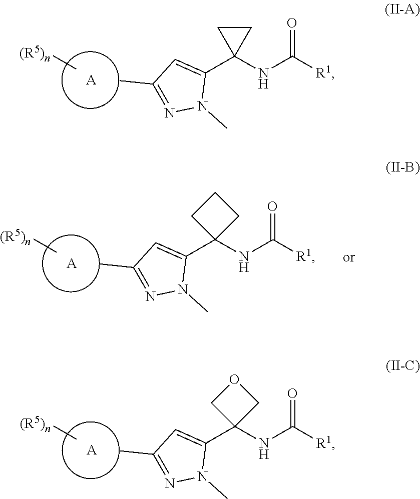

or a pharmaceutically acceptable salt thereof.

In one aspect, provided is a compound of Formula (III-A), (III-B) or (III-C) having a pyrazole core:

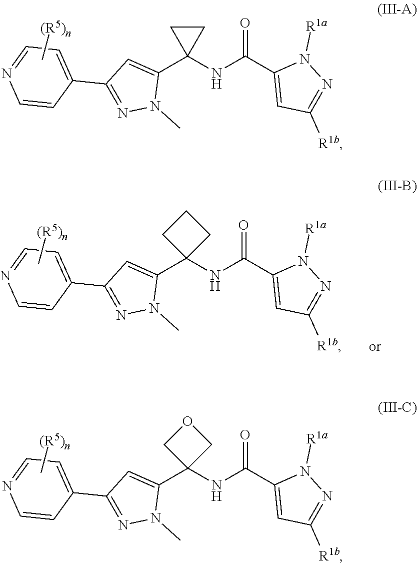

or a pharmaceutically acceptable salt thereof, wherein:
each of R^{1a} and R^{1b} is independently chosen from a C₁₋₄ alkyl, a C₁₋₄ haloalkyl, or a C₃₋₅ carbocyclyl; and
R⁵ at each occurrence is independently chosen from a C₁₋₄ alkyl, a C₁₋₄ haloalkyl, a C₁₋₄ alkoxy, or a C₃₋₅ carbocyclyl.

In another aspect, provided is a compound of Formula (III-A-i), (III-B-i), or (III-C-i) having a pyrazole core:

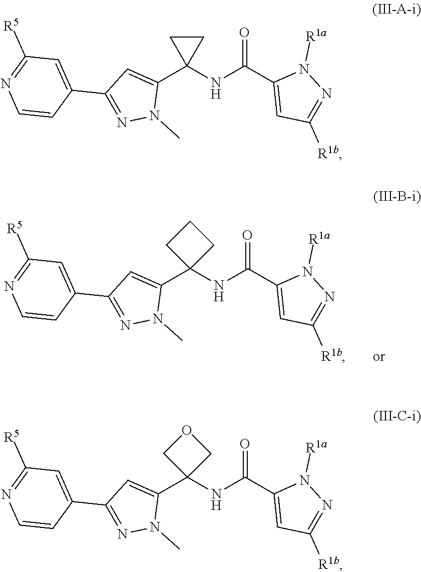

or a pharmaceutically acceptable salt thereof, wherein:
each of R^{1a} and R^{1b} is independently chosen from —CH₃, —CF₃, —C(CH₃)₃, —CHF₂, —CH(CH₃)₂, or a cyclopropyl; and
R⁵ is chosen from —CF₃, —CH₃, —CH₂CH₃, —OCH₃, —OCH₂CH₃, or a cyclopropyl.

In another aspect, provided is a compound of Formula (III-D-i) or (III-E-i):

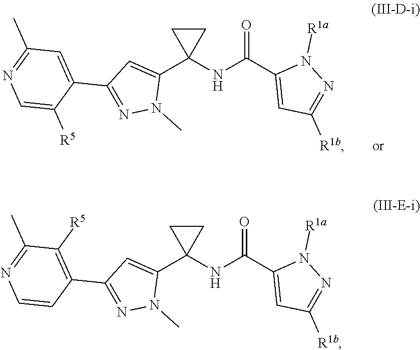

or a pharmaceutically acceptable salt thereof, wherein:
each of R^{1a} and R^{1b} is independently chosen from —CH₃, —CF₃, —C(CH₃)₃, —CHF₂, —CH(CH₃)₂, or a cyclopropyl; and
R⁵ is chosen from —F or —CN.

In some variations of Formula (III-A), (III-B) (III-C), (III-A-i), (III-B-i), (III-C-i), (III-D-i), or (III-E-i), R^{1a} is chosen from —CH₃, —C(CH₃)₃, —CHF₂, —CH(CH₃)₂, or a cyclopropyl. In other variations, R^{1a} is chosen from —CH₃, —C(CH₃)₃, or —CH(CH₃)₂. In yet other variations, R^{1a} is a cyclopropyl.

In another aspect, provided is a compound of Formula (IV) having a pyrazole core:

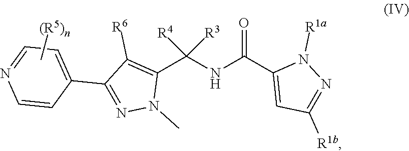

or a stereoisomer thereof, or a pharmaceutically acceptable salt thereof, wherein:
each of R^{1a} and R^{1b} is independently chosen from —CH₃, —CF₃, or —CHF₂;
R³ is —H;
R⁴ is chosen from —H or a C₁₋₄ alkyl;
n is 1 or 2.

In some embodiments of Formula (IV), R⁴ is a C₁₋₄ alkyl. In certain embodiments, R⁴ is a methyl. In some variations of the foregoing, the compound is an optically active compound. In some variations, the compound is a single enantiomer. In certain variations, the compound is the (R)-enantiomer. In other variations, the compound is the (S)-enantiomer.

In some variations of the compound of Formula (IV) when n is 1, R⁵ is chosen from —CF₃, —OCH₃, or —OCH₂CH₃. In other variations of the compound of Formula (IV) when n is 2, one R⁵ is —CH₃ and the other R⁵ is chosen from —F or —Cl; and R⁶ is chosen from —H or —CH₃.

In one aspect, provided is a compound, or a pharmaceutically acceptable salt thereof, selected from the compounds in Table A below.

TABLE A

| Compound No. | Compound Structure |
|---|---|
| 1020 | 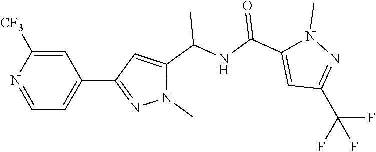 [SMILES: CC(NC(=O)c1cc(C(F)(F)F)nn1C)c1cc(-c2ccnc(C(F)(F)F)c2)nn1C] |
| 1001 | Compound 1020 Enantiomer A |
| 1002 | Compound 1020 Enantiomer B |
| 1058 | 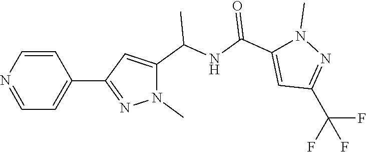 [SMILES: CC(NC(=O)c1cc(C(F)(F)F)nn1C)c1cc(-c2ccncc2)nn1C] |
| 1003 | Compound 1058 Enantiomer A |
| 1008 | Compound 1058 Enantiomer B |
| 1059 | 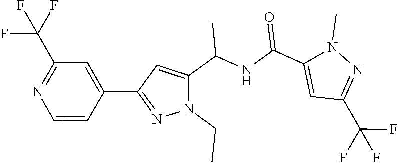 [SMILES: CCn1nc(-c2ccnc(C(F)(F)F)c2)cc1C(C)NC(=O)c1cc(C(F)(F)F)nn1C] |
| 1004 | Compound 1059 Enantiomer A |
| 1007 | Compound 1059 Enantiomer B |
| 1060 | 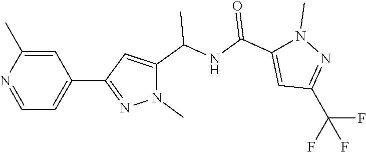 [SMILES: Cc1cc(-c2cc(C(C)NC(=O)c3cc(C(F)(F)F)nn3C)n(C)n2)ccn1] |
| 1005 | Compound 1060 Enantiomer A |
| 1006 | Compound 1060 Enantiomer B |
| 1009 | 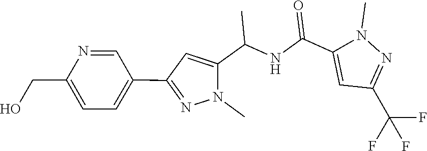 [SMILES: CC(NC(=O)c1cc(C(F)(F)F)nn1C)c1cc(-c2ccc(CO)nc2)nn1C] |
| 1061 | 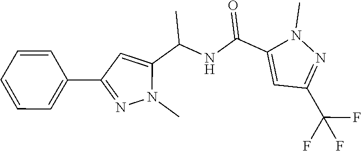 [SMILES: CC(NC(=O)c1cc(C(F)(F)F)nn1C)c1cc(-c2ccccc2)nn1C] |
| 1010 | Compound 1061 Enantiomer A |
| 1011 | Compound 1061 Enantiomer B |
| 1012 | 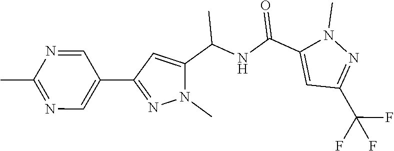 [SMILES: Cc1ncc(-c2cc(C(C)NC(=O)c3cc(C(F)(F)F)nn3C)n(C)n2)cn1] |
| 1013 | 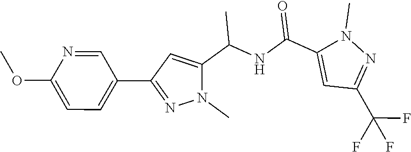 [SMILES: COc1ccc(-c2cc(C(C)NC(=O)c3cc(C(F)(F)F)nn3C)n(C)n2)cn1] |
| 1062 | 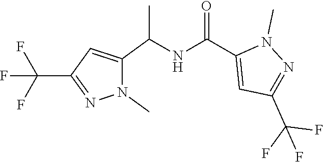 [SMILES: CC(NC(=O)c1cc(C(F)(F)F)nn1C)c1cc(C(F)(F)F)nn1C] |
| 1014 | Compound 1062 Enantiomer A |
| 1015 | Compound 1062 Enantiomer B |
| 1016 | 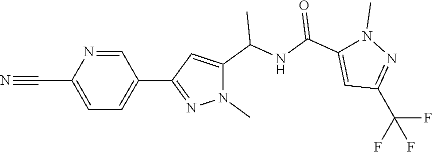 [SMILES: CC(NC(=O)c1cc(C(F)(F)F)nn1C)c1cc(-c2ccc(C#N)nc2)nn1C] |
| 1017 | 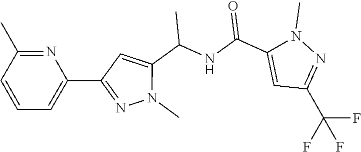 [SMILES: Cc1cccc(-c2cc(C(C)NC(=O)c3cc(C(F)(F)F)nn3C)n(C)n2)n1] |
| 1018 | 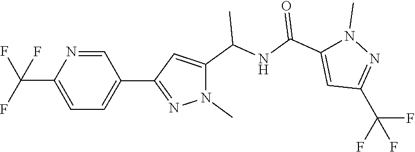 [SMILES: CC(NC(=O)c1cc(C(F)(F)F)nn1C)c1cc(-c2ccc(C(F)(F)F)nc2)nn1C] |
| 1019 | 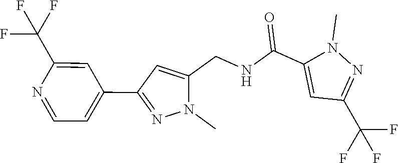 [SMILES: Cn1nc(-c2ccnc(C(F)(F)F)c2)cc1CNC(=O)c1cc(C(F)(F)F)nn1C] |
| 1021 | 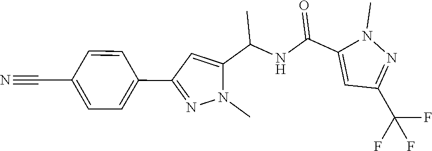 [SMILES: CC(NC(=O)c1cc(C(F)(F)F)nn1C)c1cc(-c2ccc(C#N)cc2)nn1C] |
| 1022 | 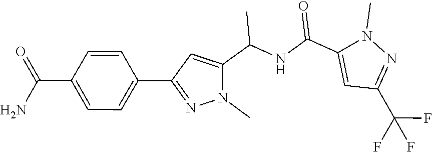 [SMILES: CC(NC(=O)c1cc(C(F)(F)F)nn1C)c1cc(-c2ccc(C(N)=O)cc2)nn1C] |
| 1023 | 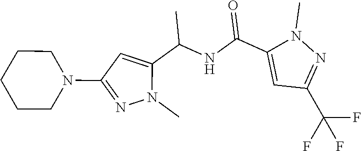 [SMILES: CC(NC(=O)c1cc(C(F)(F)F)nn1C)c1cc(N2CCCCC2)nn1C] |
| 1024 | 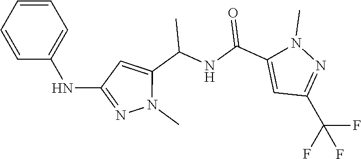 [SMILES: CC(NC(=O)c1cc(C(F)(F)F)nn1C)c1cc(Nc2ccccc2)nn1C] |
| 1025 | 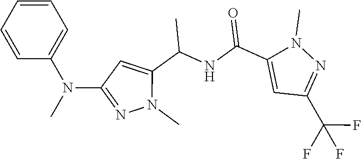 [SMILES: CC(NC(=O)c1cc(C(F)(F)F)nn1C)c1cc(N(C)c2ccccc2)nn1C] |
| 1026 | 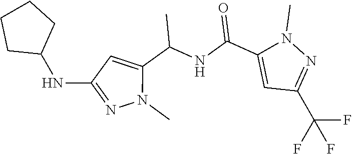 [SMILES: CC(NC(=O)c1cc(C(F)(F)F)nn1C)c1cc(NC2CCCC2)nn1C] |
| 1027 | 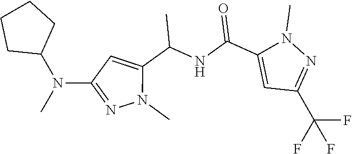 [SMILES: CC(NC(=O)c1cc(C(F)(F)F)nn1C)c1cc(N(C)C2CCCC2)nn1C] |
| 1028 | 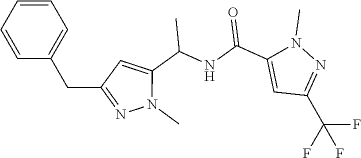 [SMILES: CC(NC(=O)c1cc(C(F)(F)F)nn1C)c1cc(Cc2ccccc2)nn1C] |
| 1029 | 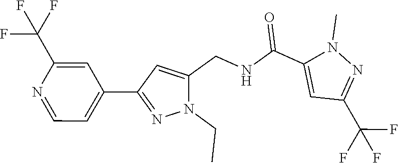 [SMILES: CCn1nc(-c2ccnc(C(F)(F)F)c2)cc1CNC(=O)c1cc(C(F)(F)F)nn1C] |
| 1030 | 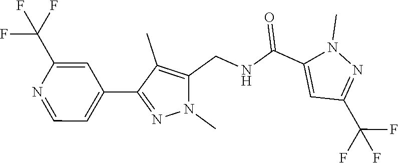 [SMILES: Cc1c(-c2ccnc(C(F)(F)F)c2)nn(C)c1CNC(=O)c1cc(C(F)(F)F)nn1C] |
| 1031 | 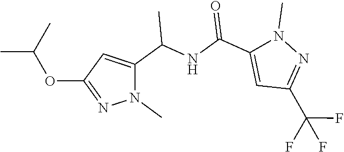 [SMILES: CC(C)Oc1cc(C(C)NC(=O)c2cc(C(F)(F)F)nn2C)n(C)n1] |
| 1032 | 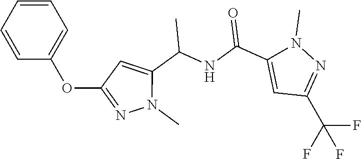 [SMILES: CC(NC(=O)c1cc(C(F)(F)F)nn1C)c1cc(Oc2ccccc2)nn1C] |
| 1033 | 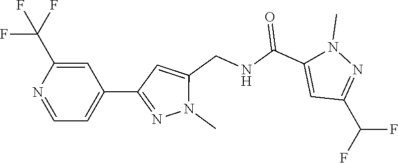 [SMILES: Cn1nc(-c2ccnc(C(F)(F)F)c2)cc1CNC(=O)c1cc(C(F)F)nn1C] |
| 1034 | 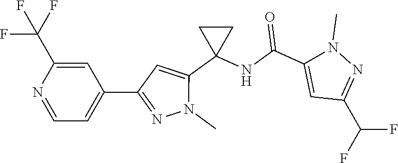 [SMILES: Cn1nc(C(F)F)cc1C(=O)NC1(c2cc(-c3ccnc(C(F)(F)F)c3)nn2C)CC1] |
| 1035 | 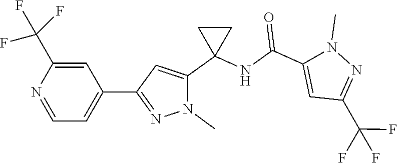 [SMILES: Cn1nc(C(F)(F)F)cc1C(=O)NC1(c2cc(-c3ccnc(C(F)(F)F)c3)nn2C)CC1] |
| 1036 | 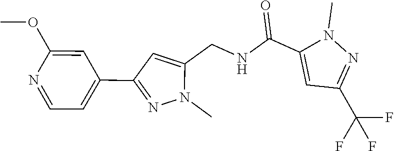 [SMILES: COc1cc(-c2cc(CNC(=O)c3cc(C(F)(F)F)nn3C)n(C)n2)ccn1] |
| 1037 | 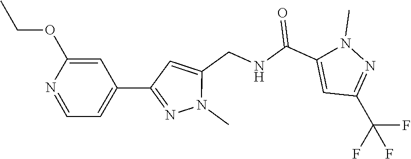 [SMILES: CCOc1cc(-c2cc(CNC(=O)c3cc(C(F)(F)F)nn3C)n(C)n2)ccn1] |
| 1038 | 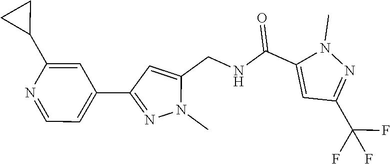 [SMILES: Cn1nc(-c2ccnc(C3CC3)c2)cc1CNC(=O)c1cc(C(F)(F)F)nn1C] |
| 1039 | 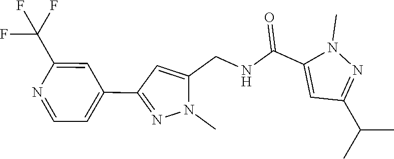 [SMILES: CC(C)c1cc(C(=O)NCc2cc(-c3ccnc(C(F)(F)F)c3)nn2C)n(C)n1] |
| 1040 | 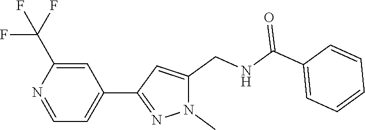 [SMILES: Cn1nc(-c2ccnc(C(F)(F)F)c2)cc1CNC(=O)c1ccccc1] |
| 1041 | 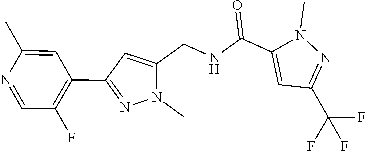 [SMILES: Cc1cc(-c2cc(CNC(=O)c3cc(C(F)(F)F)nn3C)n(C)n2)c(F)cn1] |
| 1042 | 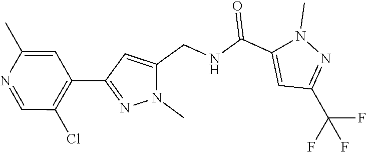 [SMILES: Cc1cc(-c2cc(CNC(=O)c3cc(C(F)(F)F)nn3C)n(C)n2)c(Cl)cn1] |
| 1043 | 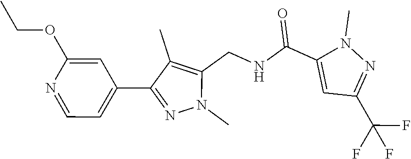 [SMILES: CCOc1cc(-c2nn(C)c(CNC(=O)c3cc(C(F)(F)F)nn3C)c2C)ccn1] |
| 1044 | 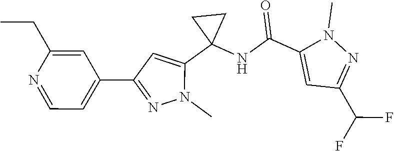 [SMILES: CCc1cc(-c2cc(C3(NC(=O)c4cc(C(F)F)nn4C)CC3)n(C)n2)ccn1] |
| 1045 | 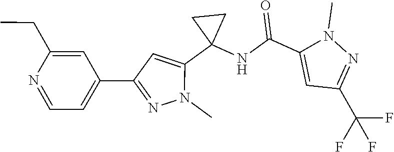 [SMILES: CCc1cc(-c2cc(C3(NC(=O)c4cc(C(F)(F)F)nn4C)CC3)n(C)n2)ccn1] |
| 1046 | 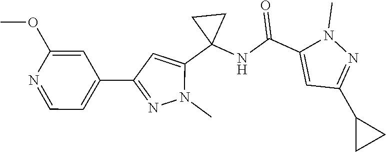 [SMILES: COc1cc(-c2cc(C3(NC(=O)c4cc(C5CC5)nn4C)CC3)n(C)n2)ccn1] |
| 1047 | 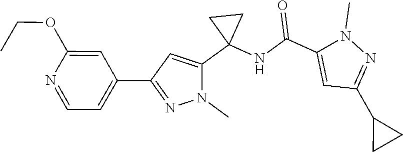 [SMILES: CCOc1cc(-c2cc(C3(NC(=O)c4cc(C5CC5)nn4C)CC3)n(C)n2)ccn1] |
| 1048 | 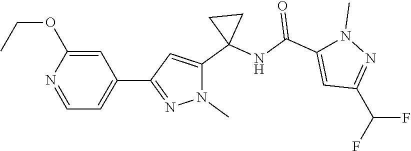 [SMILES: CCOc1cc(-c2cc(C3(NC(=O)c4cc(C(F)F)nn4C)CC3)n(C)n2)ccn1] |
| 1049 | 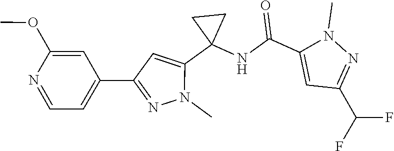 [SMILES: COc1cc(-c2cc(C3(NC(=O)c4cc(C(F)F)nn4C)CC3)n(C)n2)ccn1] |
| 1050 | 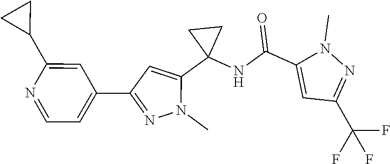 [SMILES: Cn1nc(C(F)(F)F)cc1C(=O)NC1(c2cc(-c3ccnc(C4CC4)c3)nn2C)CC1] |
| 1051 | 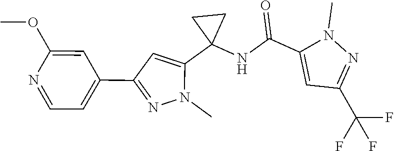 [SMILES: COc1cc(-c2cc(C3(NC(=O)c4cc(C(F)(F)F)nn4C)CC3)n(C)n2)ccn1] |
| 1052 | 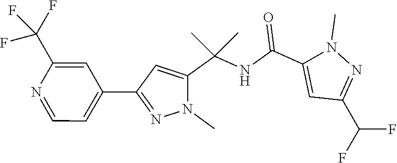 [SMILES: Cn1nc(C(F)F)cc1C(=O)NC(C)(C)c1cc(-c2ccnc(C(F)(F)F)c2)nn1C] |
| 1053 | 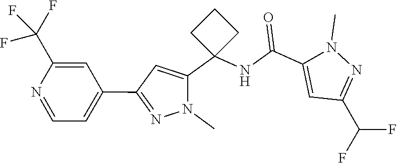 [SMILES: Cn1nc(C(F)F)cc1C(=O)NC1(c2cc(-c3ccnc(C(F)(F)F)c3)nn2C)CCC1] |
| 1054 | 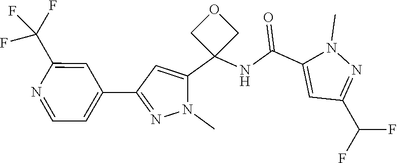 [SMILES: Cn1nc(C(F)F)cc1C(=O)NC1(c2cc(-c3ccnc(C(F)(F)F)c3)nn2C)COC1] |
| 1055 | 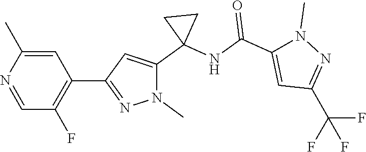 [SMILES: Cc1cc(-c2cc(C3(NC(=O)c4cc(C(F)(F)F)nn4C)CC3)n(C)n2)c(F)cn1] |
| 1056 | 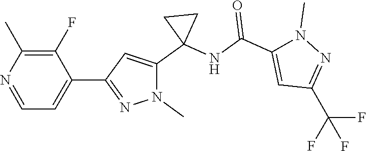 [SMILES: Cc1nccc(-c2cc(C3(NC(=O)c4cc(C(F)(F)F)nn4C)CC3)n(C)n2)c1F] |
| 1057 | 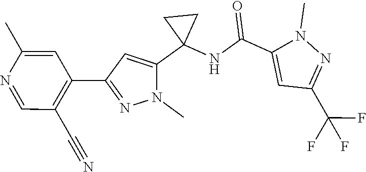 [SMILES: Cc1cc(-c2cc(C3(NC(=O)c4cc(C(F)(F)F)nn4C)CC3)n(C)n2)c(C#N)cn1] |
| 1063 | 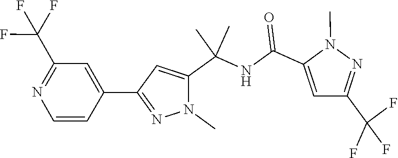 [SMILES: Cn1nc(C(F)(F)F)cc1C(=O)NC(C)(C)c1cc(-c2ccnc(C(F)(F)F)c2)nn1C] |
| 1064 | 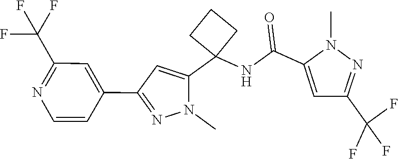 [SMILES: Cn1nc(C(F)(F)F)cc1C(=O)NC1(c2cc(-c3ccnc(C(F)(F)F)c3)nn2C)CCC1] |

In some embodiments, the compound is Compound No. 1001, 1002, 1019, 1033-1037, 1039, 1041-1051, or 1053-1057, or a pharmaceutically acceptable salt thereof. In some variations, the compound is Compound No. 1034, 1035, 1039, 1044-1051, or 1054-1057, or a pharmaceutically acceptable salt thereof.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). Embodiments disclosed herein additionally encompasses compounds described herein as individual isomers substantially free of other isomers, and alternatively, as mixtures of various isomers.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, such as more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight, or more than 99.9% by weight, of the enantiomer. In certain embodiments, the weights are based upon total weight of all enantiomers or stereoisomers of the compound.

In certain aspects, provided are compositions comprising the compounds described herein. In some embodiments, an enantiomerically pure compound can be present in the compositions with other active or inactive ingredients. For example, a pharmaceutical composition comprising enantiomerically pure R-compound can comprise, for example, about 90% excipient and about 10% enantiomerically pure R-compound. In certain embodiments, the enantiomerically pure R-compound in such compositions can, for example, comprise at least about 95% by weight R-compound and at most about 5% by weight S-compound, by total weight of the compound. For example, a pharmaceutical composition comprising enantiomerically pure S-compound can comprise, for example, about 90% excipient and about 10% enantiomerically pure S-compound. In certain embodiments, the enantiomerically pure S-compound in such compositions can, for example, comprise at least about 95% by weight S-compound and at most about 5% by weight R-compound, by total weight of the compound. In certain embodiments, the active ingredient can be formulated with little or no excipient or carrier.

Compound described herein may also comprise one or more isotopic substitutions. For example, H may be in any isotopic form, including ¹H, ²H (D or deuterium), and ³H (T or tritium); C may be in any isotopic form, including ¹²C, ¹¹C, and ¹⁴C. O may be in any isotopic form, including ¹⁶O and ¹⁸O, and F may be in any isotopic form, including ¹⁸F and ¹⁹F.

### III. Methods of Treatment

The compounds and compositions described above and herein can be used to treat a neurological disorder, a disorder associated with excessive neuronal excitability, or a disorder associated with a gain-of-function mutation in a gene (e.g., KCNT1).

In some aspects, provided are methods of treating a neurological disorder, a disorder associated with excessive neuronal excitability, or a disorder associated with a gain-of-function mutation of a gene, by administering to a subject in need thereof an effective amount of any of the compounds described herein or a pharmaceutically acceptable salt thereof, or pharmaceutical compositions comprising such compounds or a pharmaceutically acceptable salt thereof.

Exemplary diseases, disorders, or conditions include epilepsy and other encephalopathies (e.g., MMFSI or EIMFS, ADNFLE, West syndrome, infantile spasms, epileptic encephalopathy, developmental and epileptic encephalopathy (DEE), early infantile epileptic encephalopathy (EIEE), generalized epilepsy, focal epilepsy, multifocal epilepsy, temporal lobe epilepsy, Ohtahara syndrome, early myoclonic encephalopathy, Lennox-Gastaut syndrome, drug resistant epilepsy, seizures (e.g., frontal lobe seizures, generalized tonic clonic seizures, asymmetric tonic seizures, focal seizures), leukodystrophy, hypomyelinating leukodystrophy, and leukoencephalopathy), cardiac dysfunctions (e.g., cardiac arrhythmia, Brugada syndrome, myocardial infarction), pulmonary vasculopathy/hemorrhage, pain and related conditions (e.g., neuropathic pain, acute/chronic pain, migraine, etc.), muscle disorders (e.g., myotonia, neuromyotonia, cramp muscle spasms, spasticity), itch and pruritis, movement disorders (e.g., ataxia and cerebellar ataxias), psychiatric disorders (e.g., major depression, anxiety, bipolar disorder, schizophrenia, attention-deficit hyperactivity disorder), neurodevelopmental disorder, learning disorders, intellectual disability, Fragile X, neuronal plasticity, and autism spectrum disorders.

In some embodiments, the neurological disorder, the disorder associated with excessive neuronal excitability, or the disorder associated with a gain-of-function mutation in a gene (e.g., KCNT1) is selected from EIMFS, ADNFLE, or West syndrome. In some embodiments, the neurological disorder, the disorder associated with excessive neuronal excitability, or the disorder associated with a gain-of-function mutation in a gene (e.g., KCNT1) is selected from infantile spasms, epileptic encephalopathy, focal epilepsy, Ohtahara syndrome, developmental and epileptic encephalopathy, or Lennox-Gastaut syndrome. In some embodiments, the neurological disorder, the disorder associated with excessive neuronal excitability, or the disorder associated with a gain-of-function mutation in a gene (e.g., KCNT1) is seizure. In some embodiments, the neurological disorder, the disorder associated with excessive neuronal excitability, or the disorder associated with a gain-of-function mutation in a gene (e.g., KCNT1) is selected from cardiac arrhythmia, Brugada syndrome, or myocardial infarction.

In some embodiments, the neurological disorder, the disorder associated with excessive neuronal excitability, or the disorder associated with a gain-of-function mutation in a gene (e.g., KCNT1) is selected from a learning disorder, Fragile X, intellectual function, neuronal plasticity, a psychiatric disorder, or an autism spectrum disorder.

Accordingly, the compounds, pharmaceutically acceptable salts thereof, and compositions disclosed herein can be administered to a subject with a neurological disorder, a disorder associated with excessive neuronal excitability, or a disorder associated with a gain-of-function mutation in a gene such as KCNT1 (e.g., EIMFS, ADNFLE, West syndrome, infantile spasms, epileptic encephalopathy, focal epilepsy, Ohtahara syndrome, developmental and epileptic encephalopathy, Lennox-Gastaut syndrome, seizures, cardiac arrhythmia, Brugada syndrome, and myocardial infarction).

EIMFS is a rare and debilitating genetic condition characterized by an early onset (before 6 months of age) of almost continuous heterogeneous focal seizures, where seizures appear to migrate from one brain region and hemisphere to another. Patients with EIMFS are generally intellectually impaired, non-verbal and non-ambulatory. While several genes have been implicated to date, the gene that is most commonly associated with EIMFS is KCNT1. Several de novo mutations in KCNT1 have been identified in patients with EIMFS, including V271F, G288S, R428Q, R474Q, R474H, R474C, 1760M, A934T, P924L, G243S, H257D, A259D, R262Q, Q270E, L2741, F346L, C377S, R398Q, P409S, A477T, F502V, M516V, Q550del, K629E, K629N, I760F, E893K, M896K, R933G, R950Q, and K1154Q. Barcia et al. (2012) Nat Genet. 44: 1255-1260; Ishii et al. (2013) Gene 531:467-471; McTague et al. (2013) Brain. 136: 1578-1591; Epi4K Consortium & Epilepsy Phenome/Genome Project. (2013) Nature 501:217-221; Lim et al. (2016) Neurogenetics; Ohba et al. (2015) Epilepsia 56:e121-e128; Zhou et al. (2018) Genes Brain Behav. e12456; Moller et al. (2015) Epilepsia. e114-20; Numis et al. (2018) Epilepsia. 1889-1898; Madaan et al. Brain Dev. 40(3):229-232; McTague et al. (2018) Neurology. 90(1):e55-e66; Kawasaki et al. (2017) J Pediatr. 191:270-274; Kim et al. (2014) Cell Rep. 9(5):1661-1672; Ohba et al. (2015) Epilepsia. 56(9):e121-8; Rizzo et al. (2016) Mol Cell Neurosci. 72:54-63; Zhang et al. (2017) Clin Genet. 91(5):717-724; Mikati et al. (2015) Ann Neurol. 78(6):995-9; Baumer et al. (2017) Neurology. 89(21):2212; Dilena et al. (2018) Neurotherapeutics. 15(4):1112-1126. These mutations may be gain-of-function, missense mutations that are dominant (i.e., present on only one allele) and result in change-in-function of the encoded potassium channel that causes a marked increase in whole cell current when tested in Xenopus oocyte or mammalian expression systems (see e.g. Milligan et al. (2015) Ann Neurol. 75(4): 581-590; Barcia et al. (2012) Nat Genet. 44(11): 1255-1259; and Mikati et al. (2015) Ann Neurol. 78(6): 995-999).

ADNFLE has a later onset than EIMFS, generally in mid-childhood, and is generally a less severe condition. It is characterized by nocturnal frontal lobe seizures and can result in psychiatric, behavioral and cognitive disabilities in patients with the condition. While ADNFLE is associated with genes encoding several neuronal nicotinic acetylcholine receptor subunits, mutations in the KCNT1 gene have been implicated in more severe cases of the disease (Heron et al. (2012) Nat Genet. 44: 1188-1190). Functional studies of the mutated KCNT1 genes associated with ADNFLE indicated that the underlying mutations (M896I, R398Q, Y796H, and R928C) were dominant, gain-of-function mutations (Milligan et al. (2015) Ann Neurol. 75(4): 581-590; Mikati et al. (2015) Ann Neurol. 78(6): 995-999).

West syndrome is a severe form of epilepsy composed of a triad of infantile spasms, an interictal electroencephalogram (EEG) pattern termed hypsarrhythmia, and mental retardation, although a diagnosis can be made one of these elements is missing. Mutations in KCNT1, including G652V and R474H, have been associated with West syndrome (Fukuoka et al. (2017) Brain Dev 39:80-83 and Ohba et al. (2015) Epilepsia 56:e121-e128). Treatment targeting the KCNT1 channel suggests that these mutations are gain-of-function mutations (Fukuoka et al. (2017) Brain Dev 39:80-83).

In one aspect, disclosed herein is a method of treating treat a disorder associated with excessive neuronal excitability or a disorder associated with a gain-of-function mutation in a gene such as KCNT1 (for example, epilepsy and other encephalopathies (e.g., MMFSI or EIMFS), ADNFLE, West syndrome, infantile spasms, epileptic encephalopathy, focal epilepsy, Ohtahara syndrome, DEE, Lennox-Gastaut syndrome, seizures, leukodystrophy, leukoencephalopathy, intellectual disability, Multifocal Epilepsy, Generalized tonic clonic seizures, Drug resistant epilepsy, Temporal lobe epilepsy, cerebellar ataxia, Asymmetric Tonic Seizures), cardiac dysfunctions (e.g., cardiac arrhythmia, Brugada syndrome, myocardial infarction), pain and related conditions (e.g., neuropathic pain, acute/chronic pain, migraine, etc.), muscle disorders (e.g., myotonia, neuromyotonia, cramp muscle spasms, spasticity), itch and pruritis, ataxia and cerebellar ataxias, psychiatric disorders (e.g., major depression, anxiety, bipolar disorder, schizophrenia), learning disorders, Fragile X, neuronal plasticity, and autism spectrum disorders), comprising administering to a subject in need thereof a compound disclosed herein or a pharmaceutically acceptable salt thereof or a pharmaceutical composition disclosed herein.

In some examples, the subject presenting with a disorder that may be associated with a gain-of-function mutation in KCNT1 is genotyped to confirm the presence of a known gain-of-function mutation in KCNT1 prior to administration of the compounds or a pharmaceutically acceptable salt thereof or compositions disclosed herein. For example, whole exome sequencing can be performed on the subject. Gain-of-function mutations associated with EIMFS may include, but are not limited to, V271F, G288S, R428Q, R474Q, R474H, R474C, 1760M, A934T, P924L, G243S, H257D, A259D, R262Q, Q270E, L2741, F346L, C₃₇₇S, R398Q, P409S, A477T, F502V, M516V, Q550del, K629E, K629N, I760F, E893K, M896K, R933G, R950Q, and K1154Q. Gain-of-function mutations associated with ADNFLE may include, but are not limited to, M896I, R398Q, Y796H, R928C, and G288S. Gain-of-function mutations associated with West syndrome may include, but are not limited to, G652V and R474H. Gain-of-function mutations associated with temporal lobe epilepsy may include, but are not limited to, R133H and R565H. Gain-of-function mutations associated with Lennox-Gastaut may include, but are not limited to, R209C. Gain-of-function mutations associated with seizures may include, but are not limited to, A259D, G288S, R474C, and R474H. Gain-of-function mutations associated with leukodystrophy may include, but are not limited to, G288S and Q906H. Gain-of-function mutations associated with Multifocal Epilepsy may include, but are not limited to, V340M. Gain-of-function mutations associated with early-onset epilepsy (EOE) may include, but are not limited to, F346L and A934T. Gain-of-function mutations associated with Early-onset epileptic encephalopathies (EOEE) may include, but are not limited to, R428Q. Gain-of-function mutations associated with developmental and epileptic encephalopathies may include, but are not limited to, F346L, R474H, and A934T. Gain-of-function mutations associated with epileptic encephalopathies may include, but are not limited to, L437F, Y796H, P924L, and R961H. Gain-of-function mutations associated with Early Infantile Epileptic Encephalopathy (EIEE) may include, but are not limited to, M896K. Gain-of-function mutations associated with drug-resistant epilepsy and generalized tonic-clonic seizure may include, but are not limited to, F346L. Gain-of-function mutations associated with migrating partial seizures of infancy may include, but are not limited to, R428Q. Gain-of-function mutations associated with Leukoencephalopathy may include, but are not limited to, F932I. Gain-of-function mutations associated with NFLE may include, but are not limited to, A934T and R950Q. Gain-of-function mutations associated with Ohtahara syndrome may include, but are not limited to, A966T. Gain-of-function mutations associated with infantile spasms may include, but are not limited to, P924L. Gain-of-function mutations associated with Brugada Syndrome may include, but are not limited to, R¹ 106Q. Gain-of-function mutations associated with Brugada Syndrome may include, but are not limited to, R474H.

In other examples, the subject is first genotyped to identify the presence of a mutation in KCNT1, and this mutation is then confirmed to be a gain-of-function mutation using standard in vitro assays, such as those described in Milligan et al. (2015) Ann Neurol. 75(4): 581-590. Typically, the presence of a gain-of-function mutation is confirmed when the expression of the mutated KCNT1 allele results an increase in whole cell current compared to the whole cell current resulting from expression of wild-type KCNT1, as may be assessed using whole-cell electrophysiology (such as described in Milligan et al. (2015) Ann Neurol. 75(4): 581-590; Barcia et al. (2012) Nat Genet. 44(11): 1255-1259; Mikati et al. (2015) Ann Neurol. 78(6): 995-999; or Rizzo et al. Mol Cell Neurosci. (2016) 72:54-63). This increase of whole cell current can be, for example, an increase of at least or about 50%, 100%, 150%, 200%, 250%, 300%, 350%, 400%, or more. The subject can then be confirmed to have a disease or condition associated with a gain-of-function mutation in KCNT1.

In particular examples, the subject is confirmed as having a KCNT1 allele containing a gain-of-function mutation (e.g., V271F, G288S, R398Q, R428Q, R474Q, R474H, R474C, G652V, I760M, Y796H, M896I, P924L, R928C, or A934T).

The compounds or pharmaceutically acceptable salts thereof disclosed herein or the pharmaceutical composition disclosed herein (e.g., a pharmaceutical composition comprising a compound or pharmaceutically acceptable salt thereof disclosed herein, and a pharmaceutically acceptable excipient) can also be used therapeutically for conditions associated with excessive neuronal excitability where the excessive neuronal excitability is not necessarily the result of a gain-of-function mutation in KCNT1. Even in instances where the disease is not the result of increased KCNT1 expression and/or activity, inhibition of KCNT1 expression and/or activity can nonetheless result in a reduction in neuronal excitability, thereby providing a therapeutic effect. Thus, the compounds or pharmaceutically acceptable salts thereof disclosed herein or the pharmaceutical compositions disclosed herein can be used to treat a subject with conditions associated with excessive neuronal excitability, for example, epilepsy and other encephalopathies (e.g., EIMFS, ADNFLE, West syndrome, infantile spasms, epileptic encephalopathy, focal epilepsy, Ohtahara syndrome, developmental and epileptic encephalopathy, Lennox-Gastaut syndrome, seizures) or cardiac dysfunctions (e.g., cardiac arrhythmia, Brugada syndrome, myocardial infarction), regardless of whether or not the disorder is associated with a gain-of-function mutation in KCNT1.

In some variations of the foregoing, a “subject” to which administration is contemplated includes, but is not limited to, humans (i.e., a male or female of any age group, e.g., a pediatric subject (e.g., an infant, child, adolescent) or an adult subject (e.g., a young adult, middle-aged adult, or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal.

Some variations of the foregoing, “treating” or “treatment”, as used herein, contemplate an action that occurs while a subject is suffering from the specified disease, disorder or condition, which reduces the severity of the disease, disorder or condition, or retards or slows the progression of the disease, disorder or condition (also “therapeutic treatment”). In some variations, “treating” or “treatment” refers to a method or procedure for obtaining beneficial or desired results—for example, clinical results. Beneficial or desired results may include: (1) alleviating one or more symptoms caused by or associated with a disease, disorder, or condition; (2) reducing the extent of the disease, disorder, or condition; (3) slowing or stopping the development or progression of one or more symptoms caused by or associated with the disease, disorder, or condition (for example, stabilizing the disease, disorder, or condition); and (4) relieving the disease, for example, by causing the regression of one or more clinical symptoms (e.g., ameliorating the disease state, enhancing the effect of another medication, delaying or stopping the progression of the disease, increasing the quality of life, and/or prolonging survival rates).

In some variations of the foregoing, an “effective amount” of a compound or pharmaceutically acceptable salt thereof refers to an amount sufficient to elicit the desired biological response. As will be appreciated by those of ordinary skill in this art, the effective amount of a compound or pharmaceutically acceptable salt thereof may vary depending on such factors as the desired biological endpoint, the pharmacokinetics of the compound or pharmaceutically acceptable salt thereof, the disease being treated, the mode of administration, and the age, weight, health, and condition of the subject.

In some embodiments, a therapeutically effective amount of the compound or pharmaceutically acceptable salt thereof disclosed herein is administered to the subject (e.g., a human). In some variations of the foregoing, a “therapeutically effective amount” of a compound or pharmaceutically acceptable salt thereof is an amount sufficient to provide a therapeutic benefit in the treatment of a disease, disorder or condition, or to delay or minimize one or more symptoms associated with the disease, disorder or condition. A therapeutically effective amount of a compound or pharmaceutically acceptable salt thereof means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of the disease or condition, or enhances the therapeutic efficacy of another therapeutic agent.

In some embodiments, the method provided involves treating a disorder associated with a gain-of-function mutation of KCNT1. In some variations, a “disorder associated with a gain-of-function mutation in KCNT1” refers to a disorder that is associated with, is partially or completely caused by, or has one or more symptoms that are partially or completely caused by, a mutation in KCNT1 that results in a gain-of-function phenotype, i.e., an increase in activity of the potassium channel encoded by KCNT1 resulting in an increase in whole cell current. In some variations, a “gain-of-function mutation of KCNT1” is a mutation in KCNT1 that results in an increase in activity of the potassium channel encoded by KCNT1. Activity can be assessed by, for example, ion flux assay or electrophysiology (e.g., using the whole cell patch clamp technique). Typically, a gain-of-function mutation results in an increase of at least or about 20%, 30%, 40%, 50%, 60%, 70%, 80%, 90%, 100%, 125%, 150%, 175%, 200%, 225%, 250%, 275%, 300%, 325%, 350%, 375%, 400%, or more compared to the activity of a potassium channel encoded by a wild-type KCNT1.

### IV. Pharmaceutical Compositions and Routes of Administration

Compounds or pharmaceutically acceptable salts thereof provided in accordance with the present invention are usually administered in the form of pharmaceutical compositions. Therefore, disclosed herein are pharmaceutical compositions that contain, as the active ingredient, one or more of the compounds described, or a pharmaceutically acceptable salt or ester thereof, and one or more pharmaceutically acceptable excipients, carriers, including inert solid diluents and fillers, diluents, including sterile aqueous solution and various organic solvents, permeation enhancers, solubilizers and adjuvants. The pharmaceutical compositions may be administered alone or in combination with other therapeutic agents. Such compositions may be prepared in a manner disclosed in the pharmaceutical art, including, for example, in Remington's Pharmaceutical Sciences, Mace Publishing Co., Philadelphia, Pa. 17th Ed. (1985) and Modern Pharmaceutics, Marcel Dekker, Inc. 3rd Ed. (G. S. Banker & C. T. Rhodes, Eds.).

The pharmaceutical compositions may be administered in either single or multiple doses by any of the accepted modes of administration of agents having similar utilities, for example as described in those patents and patent applications incorporated by reference, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer.

One mode for administration is parenteral, particularly by injection. The forms in which the novel compositions disclosed herein may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersions, and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating a compound or pharmaceutically acceptable salt thereof as disclosed herein in the required amount in the appropriate solvent with various other ingredients as enumerated above, as desired, followed by filtered sterilization. Generally, dispersions are prepared by incorporating the various sterilized active ingredients into a sterile vehicle which contains the basic dispersion medium and the desired other ingredients from those enumerated above. In the case of sterile powders for the preparation of sterile injectable solutions, exemplary methods of preparation include vacuum-drying and freeze-drying techniques which yield a powder of the active ingredient plus any additional desired ingredient from a previously sterile-filtered solution thereof.

Oral administration is another route for administration of the compounds or pharmaceutically acceptable salts thereof as disclosed herein. Administration may be via capsule or enteric coated tablets, or the like. In making the pharmaceutical compositions that include at least one compound or pharmaceutically acceptable salt thereof described herein, the active ingredient may be diluted by an excipient and/or enclosed within such a carrier that can be in the form of a capsule, sachet, paper or other container. When the excipient serves as a diluent, it can be in the form of a solid, semi-solid, or liquid material (as above), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. In certain embodiments, the compositions disclosed herein can additionally include lubricating agents such as talc, magnesium stearate, and mineral oil; wetting agents; emulsifying and suspending agents; preserving agents such as methyl and propylhydroxy-benzoates; sweetening agents; and flavoring agents.

The compositions disclosed herein can be formulated so as to provide quick, sustained or delayed release of the active ingredient after administration to the patient by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another embodiment for use in the methods disclosed herein may employ transdermal delivery devices (“patches”). Such transdermal patches may be used to provide continuous or discontinuous infusion of the compounds or pharmaceutically acceptable salts thereof as disclosed herein in controlled amounts. The construction and use of transdermal patches for the delivery of pharmaceutical agents is described, for example, in U.S. Pat. Nos. 5,023,252, 4,992,445 and 5,001,139. Such patches may be constructed for continuous, pulsatile, or on-demand delivery of pharmaceutical agents.

The compositions disclosed herein may be formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient (e.g., a tablet, capsule, ampoule). The compounds are generally administered in a pharmaceutically effective amount. Preferably, for oral administration, each dosage unit contains from about 1 mg to about 2 g of a compound or pharmaceutically acceptable salt thereof as described herein, and for parenteral administration, preferably from about 0.1 to about 700 mg of a compound or pharmaceutically acceptable salt thereof as described herein. It will be understood, however, that the amount of the compound or pharmaceutically acceptable salt thereof actually administered usually will be determined by a physician, in the light of the relevant circumstances, including the condition to be treated, the chosen route of administration, the actual compound or pharmaceutically acceptable salt thereof administered and its relative activity, the age, weight, and response of the individual patient, the severity of the patient's symptoms, and the like.

For preparing solid compositions such as tablets, the principal active ingredient may be mixed with a pharmaceutical excipient to form a solid preformulation composition containing a homogeneous mixture of a compound or pharmaceutically acceptable salt thereof as disclosed herein. When referring to these preformulation compositions as homogeneous, it is meant that the active ingredient is dispersed evenly throughout the composition so that the composition may be readily subdivided into equally effective unit dosage forms such as tablets, pills and capsules.

The tablets or pills disclosed herein may be coated or otherwise compounded to provide a dosage form affording the advantage of prolonged action, or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described herein. In certain embodiments, the compositions are administered by the oral or nasal respiratory route for local or systemic effect. Compositions in pharmaceutically acceptable solvents may be nebulized by use of inert gases. Nebulized solutions may be inhaled directly from the nebulizing device or the nebulizing device may be attached to a facemask tent or intermittent positive pressure breathing machine. Solution, suspension, or powder compositions may be administered, such as orally or nasally, from devices that deliver the formulation in an appropriate manner.

In some embodiments, there is provided a pharmaceutical composition comprising a compound, or pharmaceutically acceptable salt thereof, as disclosed herein and at least one pharmaceutically acceptable excipient and/or carrier.

Those skilled in the art will recognize or be able to ascertain using no more than routine experimentation many equivalents to the specific embodiments described herein. The scope of the present embodiments described herein is not intended to be limited to the above Description or the Examples that follow, but rather is as set forth in the appended claims. Those of ordinary skill in the art will appreciate that various changes and modifications to this description may be made without departing from the spirit or scope of the embodiments disclosed herein, as defined in the claims.

### EXAMPLES

In order that the embodiments described herein may be more fully understood, the following examples are set forth. The synthetic and biological examples described in this application are offered to illustrate the compounds, pharmaceutical compositions, and methods provided herein and are not to be construed in any way as limiting their scope.

The compounds provided herein can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimal reaction conditions may vary with the particular reactants or solvent used, but such conditions can be determined by one skilled in the art by routine optimization.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are described in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

The compounds provided herein may be isolated and purified by known standard procedures. Such procedures include recrystallization, filtration, flash chromatography, trituration, high performance liquid chromatography (HPLC), or supercritical fluid chromatography (SFC). Note that flash chromatography may either be performed manually or via an automated system. The compounds provided herein may be characterized by known standard procedures, such as nuclear magnetic resonance spectroscopy (NMR) or liquid chromatography mass spectrometry (LCMS). NMR chemical shifts are reported in part per million (ppm) and are generated using methods described in the art.

### Abbreviations

CDCl₃ Deuterated chloroform
DCM Dichloromethane
DEAD Diethyl azodicarboxylate
DIPEA N,N-Diisopropylethylamine
DMF Dimethylformamide
DMSO Dimethylsulfoxide
DMSO-d6 Deuterated dimethylsulfoxide-d6
EtOAc Ethyl acetate
EtOH Ethanol
HATU 2-(7-Azabenzotriazol-1-yl)-N,N,N′,N′-tetramethyluronium hexafluorophosphate
IPA Isopropyl alcohol
MeOH Methanol
T3P Propanephosphonic acid anhydride
TFA Trifluoroacetic acid
THF Tetrahydrofuran

### I. Synthesis and Characterization of Exemplary Compounds

Exemplary methods for preparing compounds described herein are illustrated in the following synthetic schemes. These schemes are given for the purpose of illustration, and should not be regarded in any manner as limiting the scope or the spirit of the embodiments disclosed herein.

Certain of the exemplary compounds described herein are produced as a mixture of enantiomers. To separate the enantiomers, the mixture was purified further by preparative chiral HPLC in accordance with the analytical conditions set forth in Table 1 below, and as specified herein for a given compound.

TABLE 1

| | | | | Flow | |
|---|---|---|---|---|---|
| | | | | Rate | |
| Method | Column | Solvent A | Solvent B | (mL/min) | Isocratic/Gradient |
| A | X-Select CSH | 0.1% TFA in | ACN | 1.2 | Time(min)/B Conc.: |
| | C18 | water | | | 0.01/5, 1.0/5, |
| | (4.6 × 150 mm, | | | | 8.0/100, 12.0/100, |
| | 5 μm) | | | | 14.0/5, 18.0/5. |
| B | X-Select CSH | 0.05% formic | ACN | 1.2 | 0% B to 98% B in |
| | C18 | acid in | | | 2.0 minute, Hold |
| | (3.0 × 50 mm, | water:ACN | | | until 3.0 min, At 3.2 |
| | 2.5 μm) | (95:5) | | | min B conc is 0% |
| | | | | | up to 4.0 min. |
| C | X-Select CSH | 0.1% TFA in | ACN | 1.2 | Time(min)/B Conc.: |
| | C18 | 95:5 | | | 0.01/5, 1.0/5, |
| | (4.6 × 150 mm, | water:ACN | | | 8.0/100, 12.0/100, |
| | 3.5 μm) | | | | 14.0/5, 18.0/5. |
| D | X-Select CSH | 5 mM | ACN | 1.0 | T/B %: 0.01/20, |
| | C18 | ammonium | | | 12.0/90, 16/90 |
| | (4.6 × 150 mm, | bicarbonate in | | | |
| | 3.5 μm) | water | | | |
| E | X-Select CSH | 2.5 mM | ACN | 1.2 | 0% B to 98% B in |
| | C18 | ammonium | | | 2.0 minute, hold |
| | (3.0 × 50 mm, | bicarbonate in | | | until 3.0 min, at 3.2 |
| | 2.5 μm) | water | | | min B conc is 0% |
| | | | | | up to 4.0 min . |
| F | X-Select CSH | 0.05% formic | 0.05% | 1.2 | 0% B to 98% B in |
| | C18 | acid in | formic acid | | 2.0 minute, Hold |
| | (3.0 × 50 mm, | water: ACN | in ACN | | until 3.0 min,At 3.2 |
| | 2.5 μm) | (95:5) | | | min B conc is 0% |
| | | | | | up to 4.0 min. |
| G | X-Select CSH | 0.05% TFA in | 0.05% TFA | 1.0 | T/B % :0.01/10, |
| | C18 | 95:5 | in 5:95 | | 12/90, 16/90 |
| | (4.6 × 150 mm, | water:ACN | water:ACN | | |
| | 3.5 μm) | | | | |
| H | X-Select CSH | 0.1% formic | ACN | 1.0 | Time(min)/ B Conc.: |
| | C18 | acid in | | | 0.01/10, 6.0/90, |
| | (4.6 × 150 mm, | water: ACN | | | 10.0/100, 12.0/100, |
| | 3.5 μm) | (95:5) | | | 14.0/10, 18.0/10. |
| I | X-Select CSH | 0.1% formic | ACN | 1.0 | Time(min)/ B Conc.: |
| | C18 | acid in 95:5 | | | 0.01/10, 6.0/90, |
| | (4.6 × 150 mm, | water: ACN | | | 10.0/100, 12.0/100, |
| | 5 μm) | | | | 14/10, 18.0/10. |
| J | X-Bridge BEH | 2.5 mM | ACN | 1.2 | 0% B to 98% B in |
| | C18 | ammonium | | | 2.0 minute, hold |
| | (3 × 50 mm, | bicarbonate in | | | until 3.0 min, at 3.2 |
| | 2.5 μm) | water | | | min B conc is 0% |
| | | | | | up to 4.0 min. |
| K | X-Bridge BEH | 5 mM | ACN | 1. | T/B %: 0.01/20, |
| | C18 | ammonium | | | 12.0/90, 16/90 |
| | (4.6 × 150 mm, | bicarbonate in | | | |
| | 3.5 μm) | water | | | |
| L | X-Bridge C18 | 5 mM | ACN | 1.0 | Time(min)/ B Conc.: |
| | (4.6 × 150 mm, | ammonium | | | 0.01 Pumps Pump B |
| | 5 μm) | bicarbonate in | | | Conc. 10 6.00 Pumps |
| | | water | | | Pump B Conc. 90 |
| | | | | | 10.00 Pumps Pump |
| | | | | | B Conc. 100 12.00 |
| | | | | | Pumps Pump B |
| | | | | | Conc. 100 14.00 |
| | | | | | Pumps Pump B |
| | | | | | Conc. 10 18.00 |
| | | | | | Pumps Pump B |
| | | | | | Conc. 10 18.00 |
| | | | | | Controller Stop |
| M | Chiral Pak AD- | 0.1% DEA in | 50:50 | 1.0 | 90:10 (A:B) |
| | H | n-hexane | MeOH:EtOH | | |
| | (4.6 × 250 mm, | | | | |
| | 5 μm) | | | | |
| N | CELLULOSE-4 | 0.1% TFA in | IPA | 1.0 | 95:5 (A:B) |
| | (4.6 × 250 mm, | n-hexane | | | |
| | 5μm) | | | | |
| 0 | Chiral Pak IC | 0.1% DEA in | 8:2 | 1.0 | 95:5 (A:B) |
| | (4.6 × 250 mm, | 1:1 n- | DCM:MeOH | | |
| | 5 μm) | hexane:MTBE | | | |
| P | Chiral Pak IC | 0.1% TFA in | IPA | 0.7 | 90:10 (A:B) |
| | (4.6 × 150 mm, | n-hexane | | | |
| | 3 μm) | | | | |
| Q | Chiral Pak IC | 0.1% TFA in | IPA | 0.7 | 98:2 (A:B) |
| | (4.6 × 150 mm, | n-hexane | | | |
| | 3 μm) | | | | |

### Example 1

### Synthesis and Characterization of Exemplary Compounds Based on Synthetic Scheme A

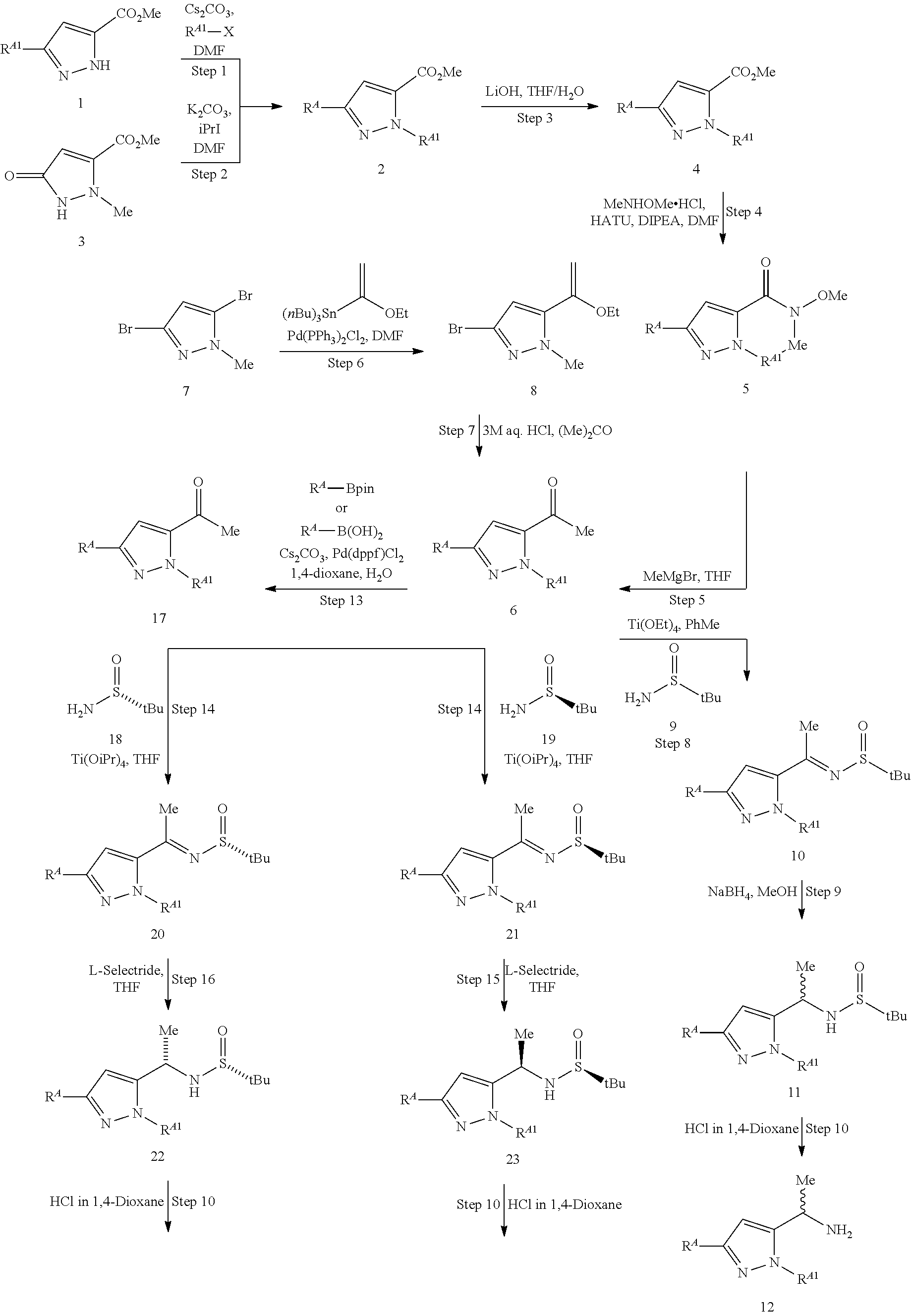

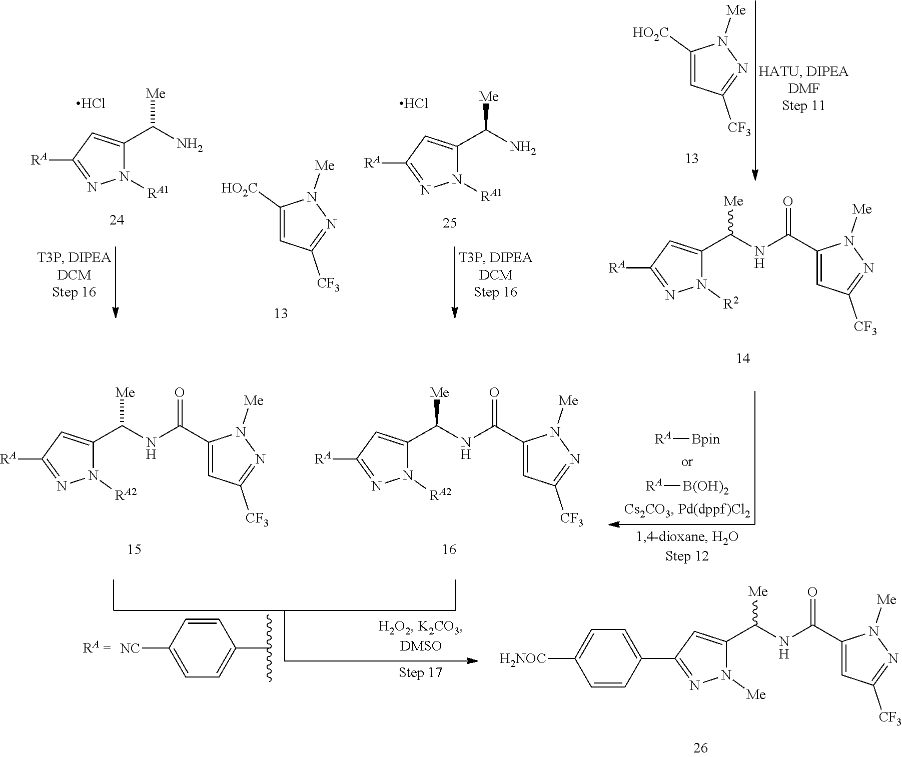

### Step 1

To a stirred solution of compound 1 (1 equiv.) in DMF (0.6 M) were added cesium carbonate (2 equiv.) and alkyl halide (1 equiv.) at about 0° C., and then stirring was continued at about 60° C. for around 12 hours. After completion of the reaction, the reaction mixture was diluted with water and extracted with ethyl acetate. The combined organic layers were dried over anhydrous sodium sulfate and concentrated under reduced pressure. The obtained crude residue was then purified by flash column chromatography, eluting with ethyl acetate/heptane mixtures, to afford compound 2.

### Step 2

In a sealed tube, to a stirred solution of compound 3 (1 equiv.) in DMF (0.64 M) were added alkyl halide (1.5 equiv.) and potassium carbonate (1.5 equiv.), and then stirring was continued at about 100° C. for around 4 hours. After completion of the reaction, the reaction mixture was diluted with water and extracted with ethyl acetate. The organic layer was washed with brine solution, dried over anhydrous sodium sulfate, and concentrated under reduced pressure to afford compound 2, which was used in Step 3 below without further purification.

### Step 3

To a stirred solution of compound 2 (1 equiv.) in a 1:3 water:THF (0.35 M) mixture was added lithium hydroxide hydrate (2 equiv.) at about 0° C., and then stirring was continued at room temperature for around 16 hours. After completion of the reaction, the organic solvent was distilled off under reduced pressure. To the crude residue was added water, which was acidified with 2 N aqueous hydrochloric acid. The precipitate was filtered to afford compound 4, which was used in Step 4 below without further purification.

### Step 4

To a stirred solution of compound 4 (1 equiv.) and N,O-dimethylhydroxylamine hydrochloride (2 equiv.) in DCM (0.8 M) were added HATU (1.5 equiv.) and DIPEA (3 equiv.) at about 0° C., and then stirring was continued at the same temperature for around 4 hours. After completion of the reaction, the reaction mixture was quenched with water and extracted with DCM. The combined organic layers were dried over anhydrous sodium sulfate and concentrated under reduced pressure. The obtained crude residue was then purified by column chromatography, eluting with ethyl acetate/hexane mixtures, to afford compound 5.

### Step 5

To a stirred solution of compound 5 (1 equiv.) in dry THF (0.2 M) was added methyl magnesium bromide solution (3 M, 2 equiv.) at about 0° C., and then stirring was continued for around 2 hours at the same temperature. After completion of the reaction, the reaction mixture was diluted with saturated aqueous ammonium chloride solution, extracted with ethyl acetate, dried over anhydrous sodium sulfate, and concentrated under reduced pressure. The obtained crude residue was then purified by column chromatography, eluting with ethyl acetate/hexane mixtures, to afford compound 6.

### Step 6

To a stirred solution of compound 7 (1 equiv.) and 1-ethoxyvinyltri-n-butyltin (1.2 equiv.) in DMF (0.4 M) was added bis(triphenylphosphine)palladium(II) dichloride (10 mol %) under a nitrogen atmosphere. After stirring at about 60° C. for around 4 hours, the reaction mixture was quenched with aqueous potassium fluoride solution, stirred for around 30 minutes, and filtered. The filtrate was extracted with ethyl acetate. The organic layer was dried over sodium sulfate, filtered, and concentrated under reduced pressure. The residue was purified by column chromatography on silica gel, eluting with ethyl acetate/petroleum ether mixtures, to afford compound 8.

### Step 7

To a stirred solution of compound 8 (1 equiv.) in acetone (0.36 M) was added 3 M aqueous hydrochloric acid (2.8 equiv.) at about 25° C. After stirring at about 25° C. for around 16 hours, the reaction was quenched with saturated aqueous sodium bicarbonate and extracted with ethyl acetate. The combined organic layer was washed with brine, dried over sodium sulfate, filtered, and concentrated under reduced pressure to afford compound 6, which was used directly in Step 8 below.

### Step 8

To a stirred solution of compound 6 (1 equiv.) and compound 9 (1.5 equiv.) in toluene (0.4 M) was added titanium (IV) isopropoxide (2 equiv.), and then stirring was continued for around 12 hours at about 80° C. After completion of the reaction, the reaction mixture was quenched with water and extracted with ethyl acetate. The organic layer was separated, dried over anhydrous sodium sulfate, and concentrated under reduced pressure. The obtained crude residue was then purified by column chromatography, eluting with ethyl acetate/hexane mixtures, to afford compound 10.

### Step 9

To a stirred solution of compound 10 (1 equiv.) in methanol (0.45 M) was added sodium borohydride (2 equiv.) portion-wise at about 0° C., and then stirring was continued for around 2 hours at room temperature. After completion of the reaction, the reaction mixture was quenched using water and extracted with ethyl acetate. The organic layer was separated, dried over anhydrous sodium sulfate, and concentrated under reduced pressure. The obtained crude residue was then purified by column chromatography, eluting with ethyl acetate/hexane mixtures, to afford compound 11.

### Step 10

To a stirred solution of compound 11 (1 equiv.) in 1,4-dioxane (1 M) was added 4 M HCl in dioxane (6 equiv.) at about 0° C., and then stirring was continued at room temperature for around 2 hours. The reaction mixture was concentrated under reduced pressure to obtain crude product which was washed with diethyl ether to afford compound 12.

### Step 11

To a stirred solution of compound 12 (1 equiv.) and compound 13 (2 equiv.) in DCM (0.2 M) were added HATU (1.5 equiv.) and DIPEA (3 equiv.) at about 0° C., and then stirring was continued at about 0° C. for around 2 hours. After completion of the reaction, the reaction mixture was quenched with water and extracted with DCM. The combined organic layers were dried over anhydrous sodium sulfate and concentrated under reduced pressure. The obtained crude residue was then purified by column chromatography, eluting with ethyl acetate/hexane mixtures, to afford compound 14.

### Step 12

To a stirred solution of compound 14 (1 equiv.) in a 1:4 water:1,4-dioxane (0.07 M) mixture were added cesium carbonate (2 equiv.) and boronic acid or boronic ester (2 equiv.) at room temperature. The reaction mixture was degassed under argon for around 5 minutes, followed by the addition of bis(triphenylphosphine)palladium(II) dichloride (5 mol %). The reaction mixture was microwaved at about 100° C. for around 1 hour. The reaction mixture was allowed cool to room temperature, filtered through a pad of celite, washing with ethyl acetate. The filtrate was washed with water, dried over anhydrous sodium sulfate, and concentrated under reduced pressure. The obtained crude residue was then purified by column chromatography, eluting with ethyl acetate, to afford compound 15 and 16, as a mixture of enantiomers.

To separate the enantiomers, the mixture was purified further by preparative chiral HPLC (CHIRAL PAK AD-H (250×4.6 mm, 5 μm) Mobile phase A: 0.1% DEA in n-Hexane; Mobile phase B: ETOH:MEOH (50:50); Program—A B 90:10; Flow rate: 1.0 ml/min) to afford compound 15 and compound 16.

### Step 13

A stirred solution of compound 6 (1 equiv.), boronic acid or boronic ester (1.3 equiv.), [1,1′-bis(diphenylphosphino)ferrocene]palladium(II) dichloride (5 mol %), and cesium carbonate (3 equiv.) in a 1:10 water:1,4-dioxane (0.35 M) mixture was stirred at about 80° C. for around 16 hours under a nitrogen atmosphere. The mixture was cooled to about 25° C., filtered, and concentrated to give the crude product, which was purified by flash chromatography on silica gel, eluting with ethyl acetate/petroleum ether mixtures to afford compound 17.

### Step 14

To a stirred solution of compound 17 (1 equiv.) in THF (0.15 M) was added rac-(R)-2-methylpropane-2-sulfinamide (18) or rac-(S)-2-methylpropane-2-sulfinamide (19) (1.5 equiv.) and titanium (IV) isopropoxide (10 equiv.) at about 25° C. under nitrogen atmosphere. The mixture was heated to about 65° C. and stirred for around 16 hours. The reaction was quenched with saturated aqueous sodium bicarbonate and filtered. The filtrate was extracted with ethyl acetate. The combined organic layer was dried over sodium sulfate, filtered, and concentrated under reduced pressure. The crude product was purified by column chromatography on silica gel, eluting with ethyl acetate/petroleum ether mixtures, to afford compound 20 or compound 21, depending on the enantiomer of sulfinamide used.

### Step 15

To a stirred solution of compound 20 or compound 21 (1 equiv.) in THF (0.13 M) was added L-Selectride (2 equiv.) at about −78° C. After stirring at about −78° C. for around 0.5 hour, the mixture was poured onto saturated aqueous ammonium chloride and extracted with ethyl acetate (×2). The combined organic layer was washed with brine, dried over anhydrous sodium sulfate, filtered, and concentrated. The residue was purified by flash column chromatography, eluting with 0-10% MeOH/DCM, to afford compound 22 or compound 23.

### Step 16

To a stirred solution of compound 13 (1.2 equiv.) in DCM (0.05 M) was added DIPEA (8 equiv.) and T3P (3 equiv.). After stirring at about 25° C. for around 30 minutes, compound 24 or compound 25 (1 equiv.) was added, and the reaction was stirred at about 25° C. for around 16 hours. The reaction was quenched with water and extracted with DCM. The combined organic layer was washed with brine, dried over sodium sulfate, filtered, and concentrated. The residue was purified by flash column chromatography, eluting with ethyl acetate/petroleum ether mixtures. The material was further purified by Supercritical Fluid Chromatography (Column: DAICEL CHIRALCEL OJ (250 mm×30 mm, 10 m); Condition: 0.1% NH₃H₂O-EtOH; Begin B: 15; End B: 15), to afford compound 15 or compound 16.

### Step 17

To a stirred solution of compound 15 and compound 16 (1 equiv.) in DMSO (0.24 M) was added potassium carbonate (3 equiv.) followed by 30% hydrogen peroxide (10 equiv.) at room temperature, and then stirring was continued at room temperature for around 12 hours. After the starting material was observed to be consumed, the reaction was quenched by adding water and was extracted with ethyl acetate. The combined organic layers were washed with water and brine. The organic layer was separated, dried over anhydrous sodium sulfate, and concentrated under reduced pressure. The obtained crude residue was then purified by column chromatography, eluting with 50-60% EtOAc:heptane, to afford compound 1022.

Compounds 1001-1018, 1020-1022, and 1031 were prepared from commercially available starting materials, and generally in accordance with Synthetic Scheme A and steps set forth above.

Exemplary compounds prepared in accordance with Synthetic Scheme A were characterized by NMR, HPLC, and LCMS. The data for NMR and LCMS, as well as the chiral separation method (where applicable) are provided in Table 2 below. For Compound Nos. 1001 and 1002, the following SFC chiral method was used: Column: Chiralcel OJ-3 150×4.6 mm I.D., 3 m. Mobile phase: A: CO₂ B: ethanol (0.05% DEA). Gradient: from 5% to 40% of B in 5 minutes and from 40% to 50% of B in 0.5 minutes, hold 5% of B for 1.5 minutes. Flow rate: 2.5 mL/min. Column temperature: 35° C. ABPR: 1500 psi.

TABLE 2

| Compound | | |
|---|---|---|
| No. | NMR | LCMS and Chiral Separation |
| 1001 | ¹H NMR (CDCl₃ 400 MHz) δ = | MS ESI calcd |
| | 8.64 (d, J = 5.2 Hz, 1H), 7.99 (s, | for C₁₈H₁₇F₆N₆O [M + H] + 447.1, |
| | 1H), 7.80 (d, J = 4.8 Hz, 1H), | found 447.1 |
| | 6.84 (s, 1H), 6.63 (s, 1H), 6.21- | 99.95% ee |
| | 6.32 (m, 1H), 5.47 (s, 1H), 4.25 | |
| | (s, 3H), 3.96 (s, 3H), 1.70 (d, J = | |
| | 7.2 Hz, 3H). | |
| 1002 | ¹H NMR (CDCl₃ 400 MHz) δ = | MS ESI calcd |
| | 8.60 (d, J = 5.2 Hz, 1H), 7.96 (s, | for C₁₈H₁₇F₆N₆O [M + H]⁺ 447.1, |
| | 1H), 7.79 (d, J = 5.2 Hz, 1H), | found 446.8 |
| | 6.88 (s, 1H), 6.60 (s, 1H), 6.47- | 99.98% ee |
| | 6.31 (m, 1H), 5.57-5.34 (m, 1H), | |
| | 4.26 (s, 3H), 3.95 (s, 3H), 1.69 | |
| | (d, J = 6.8 Hz, 3H); ¹⁹F NMR | |
| | (376.5 MHz, CDCl₃) δF = −62.261, −67.952. | |
| 1003 | ¹H NMR (400 MHz, DMSO-d₆) | B LCMS: 379.3 (M + H), Rt 1.749 |
| | δ 9.07 (d, J = 7.8 Hz, 1H), 8.56 | min, 97.93% |
| | (d, J = 6.0 Hz, 2H), 7.76-7.71 | M Chiral method: Rt 5.875min, |
| | (m, 2H), 7.41 (s, 1H), 6.95 (s, | 99.80% |
| | 1H), 5.32 (quin, J = 7.1 Hz, 1H), | |
| | 4.14 (s, 3H), 3.86 (s, 3H), 1.57 | |
| | (d, J = 6.9 Hz, 3H). | |
| 1004 | ¹H NMR (400 MHz, DMSO-d₆): | B LCMS: 461.00 (M + H), Rt 2.056 |
| | δ 9.11 (d, J = 7.8 Hz, 1H), 8.76 | min, 99.81% |
| | (d, J = 5.1 Hz, 1H), 8.20 (s, 1H), | N Chiral method: Rt 9.25 min, |
| | 8.07 (d, J = 5.0 Hz, 1H), 7.40 (s, | 100%. |
| | 1H), 7.16 (s, 1H), 5.41-5.31 (m, | |
| | 1H), 4.20 (dq, J = 1.9, 7.2 Hz, | |
| | 2H), 4.15 (s, 3H), 1.58 (d, J = 6.8 | |
| | Hz, 3H), 1.38 (t, J = 7.2 Hz, 3H). | |
| 1005 | ¹H NMR (400 MHz, | B LCMS: 393.1 (M + H), Rt 1.408 |
| | CHLOROFORM-d) δ 8.45 (d, J = | min, 99.73% |
| | 5.3 Hz, 1H), 7.51 (s, 1H), 7.40 | O Chiral method: Rt 11.996 min, |
| | (d, J = 5.0 Hz, 1H), 6.84 (s, 1H), | 98.55%. |
| | 6.57 (s, 1H), 6.38 (br d, J = 8.3 | |
| | Hz, 1H), 5.52-5.39 (m, 1H), | |
| | 4.25 (s, 3H), 3.93 (s, 3H), 2.57 | |
| | (s, 3H), 1.69 (d, J = 7.0 Hz, 3H). | |
| 1006 | ¹H NMR (400 MHz, DMSO-d₆) | B LCMS: 393.0 (M + H), Rt 1.398 |
| | δ 9.06 (d, J = 8 Hz, 1H), 8.42 (d, | min, 99.98% |
| | J = 8 Hz, 1H), 7.62 (s, 1H), 7.54 | O Chiral method: Rt 10.328 min, |
| | (d, J = 4.0 Hz, 1H), 7.41 (s, 1H), | 99.87%. |
| | 6.91 (s, 1H), 5.32 (q, J = 6 Hz, | |
| | 1H), 4.14 (s, 3H), 3.85 (s, 3H), | |
| | 1.56 (d, J = 8.0 Hz, 3H). (3H of | |
| | aromatic methyl group might be | |
| | merged with DMSO solvent | |
| | residual peak at δ 2.50). | |
| 1007 | ¹H NMR (400 MHz, DMSO-d₆) | B LCMS: 460.90 (M + H), Rt 2.055 |
| | δ 9.15 (br d, J = 7.9 Hz, 1H), | min, 95.697% |
| | 8.77 (d, J = 5.1 Hz, 1H), 8.22 (s, | N Chiral method: Rt 10.920 min, |
| | 1H), 8.09 (d, J = 5.1 Hz, 1H), | 97.36% |
| | 7.43 (s, 1H), 7.18 (s, 1H), 5.38 | |
| | (quin, J = 7.2 Hz, 1H), 4.22 (dq, | |
| | J = 2.0, 7.2 Hz, 2H), 4.16 (s, 3H), | |
| | 1.60 (d, J = 6.8 Hz, 3H), 1.39 (t, | |
| | J = 7.2 Hz, 3H). | |
| 1008 | ¹H NMR (400 MHz, DMSO-d₆) | B LCMS: 379.0 (M + H), Rt 1.470 |
| | δ 9.11 (br d, J = 7.5 Hz, 1H), | min, 99.813% |
| | 8.75 (br d, J = 6.4 Hz, 2H), 8.11 | M Chiral method: Rt 7.521min, |
| | (br d, J = 5.6 Hz, 2H), 7.43 (s, | 99.45% |
| | 1H), 7.17 (s, 1H), 5.35 (quin, J = | |
| | 7.2 Hz, 1H), 4.16 (s, 3H), 3.93 (s, | |
| | 3H), 1.59 (br d, J = 6.8 Hz, 3H). | |
| 1009 | ¹H NMR (400 MHz, DMSO-d₆) | B LCMS: 408.9 (M + H), Rt 1.338 |
| | δ 9.08 (br d, J = 7.7 Hz, 1H), | min, 95.384% |
| | 8.91 (s, 1H), 8.15 (d, J = 7.6 Hz, | |
| | 1H), 7.50 (br d, J = 8.1 Hz, 1H), | |
| | 7.43 (s, 1H), 6.86 (s, 1H), 5.42 (t, | |
| | J = 5.7 Hz, 1H), 5.34 (t, J = 7.2 | |
| | Hz, 1H), 4.58 (d, J = 5.6 Hz, | |
| | 2H), 4.16 (s, 3H), 3.85 (s, 3H), | |
| | 1.58 (br d, J = 6.6 Hz, 3H). | |
| 1010 | ¹H NMR (400 MHz, DMSO-d₆) | B LCMS: 377.9 (M + H), Rt 1.936 |
| | δ 9.06 (br d, J = 7.9 Hz, 1H), | min, 99.546% |
| | 7.78 (br d, J = 7.2 Hz, 2H), 7.46- | P Chiral method: Rt 8.661 min, |
| | 7.24 (m, 4H), 6.75 (s, 1H), 5.31 | 99.699%. |
| | (quin, J = 7.0 Hz, 1H), 4.15 (s, | |
| | 3H), 3.82 (s, 3H), 1.56 (br d, J = | |
| | 6.8 Hz, 3H). | |
| 1011 | ¹H NMR (400 MHz, DMSO-d₆) | B LCMS: 377.9 (M + H), Rt 1.955 |
| | δ 9.06 (d, J = 7.9 Hz, 1H), 7.81- | min, 99.849% |
| | 7.74 (m, 2H), 7.44-7.34 (m, | P Chiral method: Rt 11.846min, |
| | 3H), 7.28 (d, J = 7.3 Hz, 1H), | 98.952%. |
| | 6.75 (s, 1H), 5.31 (quin, J = 7.1 | |
| | Hz, 1H), 4.15 (s, 3H), 3.82 (s, | |
| | 3H), 1.56 (d, J = 6.8 Hz, 3H). | |
| 1012 | ¹H NMR (400 MHz, DMSO-d₆) | B LCMS: 394.0 (M + H), Rt 1.756 |
| | δ 9.10-9.02 (m, 3H), 7.41 (s, | min, 99.23% |
| | 1H), 6.93 (s, 1H), 5.37-5.27 (m, | |
| | 1H), 4.14 (s, 3H), 3.85 (s, 3H), | |
| | 2.63 (s, 3H), 1.56 (br d, J = 6.8 | |
| | Hz, 3H). | |
| 1013 | ¹H NMR (400 MHz, DMSO-d₆) | B LCMS: 409.1 (M + H), Rt 1.840 |
| | δ 9.06 (d, J = 7.8 Hz, 1H), 8.56 | min, 99.695% |
| | (d, J = 1.9 Hz, 1H), 8.06 (dd, J = | |
| | 2.4, 8.6 Hz, 1H), 7.41 (s, 1H), | |
| | 6.85 (d, J = 8.6 Hz, 1H), 6.75 (s, | |
| | 1H), 5.35-5.26 (m, 1H), 4.14 (s, | |
| | 3H), 3.87 (s, 3H), 3.81 (s, 3H), | |
| | 1.55 (d, J = 6.9 Hz, 3H). | |
| 1014 | ¹H NMR (400 MHz, DMSO-d₆) | J LCMS: 368.3 (M − H), Rt 1.885 |
| | δ 9.47 (d, J = 7.6 Hz, 1H), 7.83 | min, 99.653% |
| | (s, 1H), 6.75 (s, 1H), 5.32 (q, J = | Q Chiral method: Rt 8.028 min, |
| | 7.2 Hz, 1H), 4.13 (s, 3H), 3.87 (s, | 99.65% |
| | 3H), 1.52 (d, J = 6.8 Hz, 3H). | |
| 1015 | ¹H NMR (400 MHz, DMSO-d₆) | J LCMS: 368.2 (M − H), Rt 1.884 |
| | δ 9.03 (d, J = 7.6 Hz, 1H), 7.38 | min, 99.580% |
| | (s, 1H), 6.75 (s, 1H), 5.32 (q, J = | Q Chiral method: Rt 9.698 min, |
| | 7.2 Hz, 1H), 4.13 (s, 3H), 3.87 (s, | 98.86% |
| | 3H), 1.52 (d, J = 6.8 Hz, 3H). | |
| 1016 | ¹H NMR (400 MHz, DMSO-d₆) | B LCMS: 404.1 (M + H), Rt 2.071 |
| | δ 9.17 (d, J = 1.3 Hz, 1H), 9.09 | min, 99.653% |
| | (d, J = 7.8 Hz, 1H), 8.37 (dd, J = | |
| | 2.2, 8.2 Hz, 1H), 8.05 (dd, J = | |
| | 0.6, 8.2 Hz, 1H), 7.41 (s, 1H), | |
| | 7.06 (s, 1H), 5.33 (quin, J = 7.0 | |
| | Hz, 1H), 4.14 (s, 3H), 3.88 (s, | |
| | 3H), 1.57 (d, J = 6.8 Hz, 3H). | |
| 1017 | ¹H NMR (400 MHz, DMSO-d₆) | B LCMS: 393.1 (M + H), Rt 1.831 |
| | δ 9.04 (d, J = 7.8 Hz, 1H), 7.68 | min, 98.712% |
| | (d, J = 4.8 Hz, 2H), 7.41 (s, 1H), | |
| | 7.13 (quin, J = 4.4 Hz, 1H), 6.83 | |
| | (s, 1H), 5.37-5.28 (m, 1H), 4.14 | |
| | (s, 3H), 3.86 (s, 3H), 2.48 (s, | |
| | 3H), 1.55 (d, J = 7.0 Hz, 3H). | |
| 1018 | ¹H NMR (400 MHz, DMSO-d₆) | B LCMS: 446.95 (M + H), Rt 1.950 |
| | δ 9.17 (s, 1H), 9.09 (d, J = 7.9 | min, 99.160% |
| | Hz, 1H), 8.41 (dd, J = 1.5, 8.1 | |
| | Hz, 1H), 7.92 (d, J = 8.3 Hz, | |
| | 1H), 7.41 (s, 1H), 7.03 (s, 1H), | |
| | 5.34 (quin, J = 7.1 Hz, 1H), 4.14 | |
| | (s, 3H), 3.88 (s, 3H), 1.58 (d, J = | |
| | 6.9 Hz, 3H). | |
| 1020 | ¹H NMR (400 MHz, DMSO-d₆) | ELCMS: 447.1 (M + H), Rt 2.429 |
| | δ 9.08 (d, J = 7.2 Hz, 1H), 8.75 | min, 99.150% |
| | (d, J = 4.4 Hz, 1H), 8.19 (s, 1H), | |
| | 8.07 (d, J = 4.4 Hz, 1H), 7.41 (s, | |
| | 1H), 7.17 (s, 1H), 5.34 (quin, J = | |
| | 7.1 Hz, 1H), 4.14 (s, 3H), 3.88 (s, | |
| | 3H), 1.57 (d, J = 6 Hz, 3H). | |
| 1021 | ¹H NMR (400 MHz, DMSO-d₆) | E LCMS: 403.1 (M + H), Rt 1.988 |
| | δ 9.07 (d, J = 7.9 Hz, 1H), 7.98 | min, 98.980% |
| | (d, J = 8.7 Hz, 2H), 7.84 (d, J = | |
| | 8.6 Hz, 2H), 7.41 (s, 1H), 6.93 (s, | |
| | 1H), 5.32 (quin, J = 7.1 Hz, 1H), | |
| | 4.14 (s, 3H), 3.85 (s, 3H), 1.56 | |
| | (d, J = 6.8 Hz, 3H). | |
| 1022 | ¹H NMR (400 MHz, DMSO-d₆) | E LCMS: 421.2 (M + H), Rt 1.773 |
| | δ 9.07 (d, J = 7.8 Hz, 1H), 7.96 | min, 98.669% |
| | (br s, 1H), 7.97 (q, J = 8.8 Hz, | |
| | 4H), 7.42 (s, 1H), 7.33 (br s, 1H), | |
| | 6.85 (s, 1H), 5.32 (quin, J = 7.1 | |
| | Hz, 1H), 4.15 (s, 3H), 3.83 (s, | |
| | 3H), 1.56 (d, J = 6.8 Hz, 3H). | |
| 1031 | ¹H NMR (400 MHz, DMSO-d₆) | FLCMS: 359.95 (M + H), Rt 1.961 |
| | δ 8.94 (br d, J = 7.8 Hz, 1H), | min, 99.00% |
| | 7.36 (s, 1H), 5.61 (s, 1H), 5.19- | |
| | 5.11 (m, 1H), 4.61-4.52 (m, | |
| | 1H), 4.10 (s, 3H), 3.56 (s, 3H), | |
| | 1.41 (d, J = 6.8 Hz, 3H), 1.20 (d, | |
| | J = 5.9 Hz, 6H). | |

### Example 2

### Synthesis and Characterization of Exemplary Compounds Based on Synthetic Scheme B

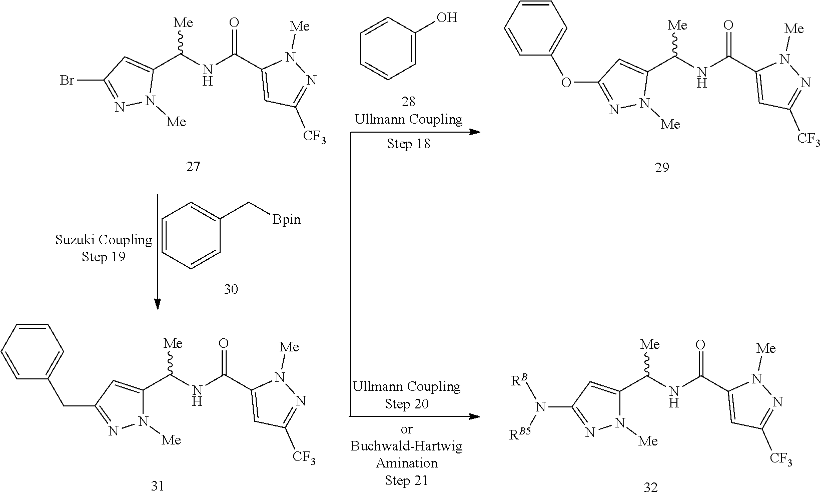

### Step 18

In a sealed tube, to a stirred solution of compound 27 (1 equiv.) in 1,4-dioxane (0.1 M) were added phenol 28 (2 equiv.), N,N-dimethyl glycine (0.6 equiv.), copper iodide (20 mol %), and cesium carbonate (2 equiv.), and then stirring was continued at around 130° C. for about 4 hours. After completion of reaction, water was added, and the reaction mixture was extracted with ethyl acetate. The combined organic layers were washed with brine solution, dried over anhydrous sodium sulfate, and concentrated under reduced pressure. The obtained crude residue was then purified by Combi-flash chromatography, eluting with 40% ethyl acetate/heptane, to afford compound 29.

### Step 19

To a stirred solution of compound 27 (400 mg, 1.05 mmol) in 1,4-dioxane (0.13 M) were added compound 30 (3 equiv.) and aqueous potassium carbonate (2 equiv.) at room temperature. The reaction mixture was purged with argon for around 10 minutes. To this resultant solution was added bis(diphenylphosphino)ferrocene]palladium(II) dichloride (10 mol %), and the solution stirred at about 100° C. for around 12 hours. The reaction mixture was allowed to cool to room temperature and filtered through a pad of celite, which was washed with ethyl acetate. The organic layer was washed with water, dried over anhydrous sodium sulfate, and concentrated under reduced pressure. The obtained crude residue was then purified by column chromatography, eluting with 50-60% EtOAc/heptane, followed by achiral prep to afford compound 31.

### Step 20

To a stirred solution of compound 27 (1 equiv.) and amine compound (2 equiv.) in DMSO (0.27 M) were added L-proline (40 mol %) and copper(I) iodide (20 mol %) at room temperature, and then stirring was continued at about 90° C. for around 16 hours. The reaction mixture was allowed to cool to room temperature and filtered through a pad of celite, which was washed with ethyl acetate. The organic layer was washed with water, dried over anhydrous sodium sulfate, and concentrated under reduced pressure. The obtained crude residue was then purified by column chromatography, eluting with ethyl acetate/heptane mixtures, followed by achiral prep to afford compound 32.

### Step 21

To a stirred solution of compound 27 (1 equiv.) in 1,4-dioxane (0.1 M) were added sodium tert-butoxide (2 equiv.) and amine compound (2 equiv.) at room temperature. The reaction mixture was purged with argon for around 10 minutes. To this solution, tris(dibenzylideneacetone)dipalladium(0) (10 mol %) and Brettphos (20 mol %) were added, and the solution was stirred at about 130° C. for around 16 hours. The reaction mixture was allowed to cool to room temperature and filtered through a pad of celite, which was washed with ethyl acetate. The organic layer was washed with water, dried over anhydrous sodium sulfate, and concentrated under reduced pressure. The obtained crude residue was then purified by column chromatography, eluting with ethyl acetate/heptane mixtures, to afford compound 32.

Compounds 1023-1028 and 1032 were prepared from commercially available starting materials, and generally in accordance with Synthetic Scheme B and steps set forth above.

Exemplary compounds prepared in accordance with Synthetic Scheme B were characterized by NMR, HPLC, and LCMS. The data for NMR and LCMS are provided in Table 3 below.

TABLE 3

| Compound | | |
|---|---|---|
| No. | NMR | LCMS |
| 1023 | ¹H NMR (400 MHz, DMSO-d₆) δ | B LCMS: 385.1 (M + H), Rt 1.936 |
| | 8.93 (br d, J = 8.2 Hz, 1H), 7.37 | min, 99.00% |
| | (s, 1H), 5.67 (s, 1H), 5.18-5.09 | |
| | (m, 1H), 4.10 (s, 3H), 3.53 (s, | |
| | 3H), 3.04-2.95 (m, 4H), 1.55- | |
| | 1.38 (m, 9H). | |
| 1024 | ¹H NMR (400 MHz, DMSO-d₆) δ | B LCMS: 393.1 (M + H), Rt 1.968 |
| | 9.01 (d, J = 8.0 Hz, 1H), 8.33 (s, | min, 99.61% |
| | 1H), 7.40 (s, 1H), 7.30 (d, J = 7.8 | |
| | Hz, 2H), 7.15 (t, J = 7.9 Hz, 2H), | |
| | 6.69 (t, J = 7.3 Hz, 1H), 5.82 (s, | |
| | 1H), 5.23 (quin, J = 7.2 Hz, 1H), | |
| | 4.14 (s, 3H), 3.67 (s, 3H), 1.49 (d, | |
| | J = 6.9 Hz, 3H). | |
| 1025 | ¹H NMR (400 MHz, DMSO-d₆) δ | B LCMS: 406.9 (M + H), Rt 2.089 |
| | 8.97 (br d, J = 8.1 Hz, 1H), 7.38 | min, 97.03% |
| | (s, 1H), 7.24 (t, J = 7.9 Hz, 2H), | |
| | 7.12 (br d, J = 7.9 Hz, 2H), 6.85 | |
| | (t, J = 7.2 Hz, 1H), 5.93 (s, 1H), | |
| | 5.24 (quin, J = 7.2 Hz, 1H), 4.14 | |
| | (s, 3H), 3.68 (s, 3H), 3.24 (s, 3H), | |
| | 1.47 (d, J = 6.9 Hz, 3H). | |
| 1026 | ¹H NMR (400 MHz, CDCl₃): δ | F LCMS: 385.0 (M + H), Rt 1.806 |
| | 6.88 (s, 1H), 6.52 (br d, J = 8.1 | min, 99.15% |
| | Hz, 1H), 5.55 (s, 1H), 5.33-5.21 | |
| | (m, 1H), 4.23 (s, 3H), 3.77-3.71 | |
| | (m, 3H), 3.69-3.62 (m, 1H), 1.99- | |
| | 1.84 (m, 2H), 1.79-1.67 (m, | |
| | 2H), 1.65-1.49 (m, 7H), 1H | |
| | merged in solvent peak. | |
| 1027 | ¹H NMR (400 MHz, DMSO-d₆) δ | F LCMS: 399.1 (M + H), Rt 1.741 |
| | 8.94 (d, J = 8.1 Hz, 1H), 7.40 (s, | min, 99.71% |
| | 1H), 5.65 (s, 1H), 5.17 (quin, J = | |
| | 7.2 Hz, 1H), 4.13 (s, 3H), 4.11- | |
| | 4.04 (m, 1H), 3.56 (s, 3H), 2.60 (s, | |
| | 3H), 1.75-1.42 (m, 11H). | |
| 1028 | ¹H NMR (400 MHz, DMSO-d₆) δ | F LCMS: 392.0 (M + H), Rt 2.066 |
| | 8.95 (br d, J = 7.8 Hz, 1H), 7.36 | min, 98.495% |
| | (s, 1H), 7.32-7.22 (m, 4H), 7.21- | |
| | 7.14 (m, 1H), 6.02 (s, 1H), 5.23 | |
| | (quin, J = 7.1 Hz, 1H), 4.12 (s, | |
| | 3H), 3.81 (s, 2H), 3.70 (s, 3H), | |
| | 1.44 (d, J = 6.8 Hz, 3H). | |
| 1032 | ¹H NMR (400 MHz, DMSO-d₆) δ | F LCMS: 393.95 (M + H), Rt 2.051 |
| | 9.01 (br d, J = 7.8 Hz, 1H), 7.41- | min, 97.37% |
| | 7.31 (m, 3H), 7.14-7.04 (m, 3H), | |
| | 5.88 (s, 1H), 5.26 (quin, J = 7.1 | |
| | Hz, 1H), 4.14 (s, 3H), 3.68 (s, | |
| | 3H), 1.47 (d, J = 6.8 Hz, 3H). | |

### Example 3

### Synthesis and Characterization of Exemplary Compounds Based on Synthetic Scheme C

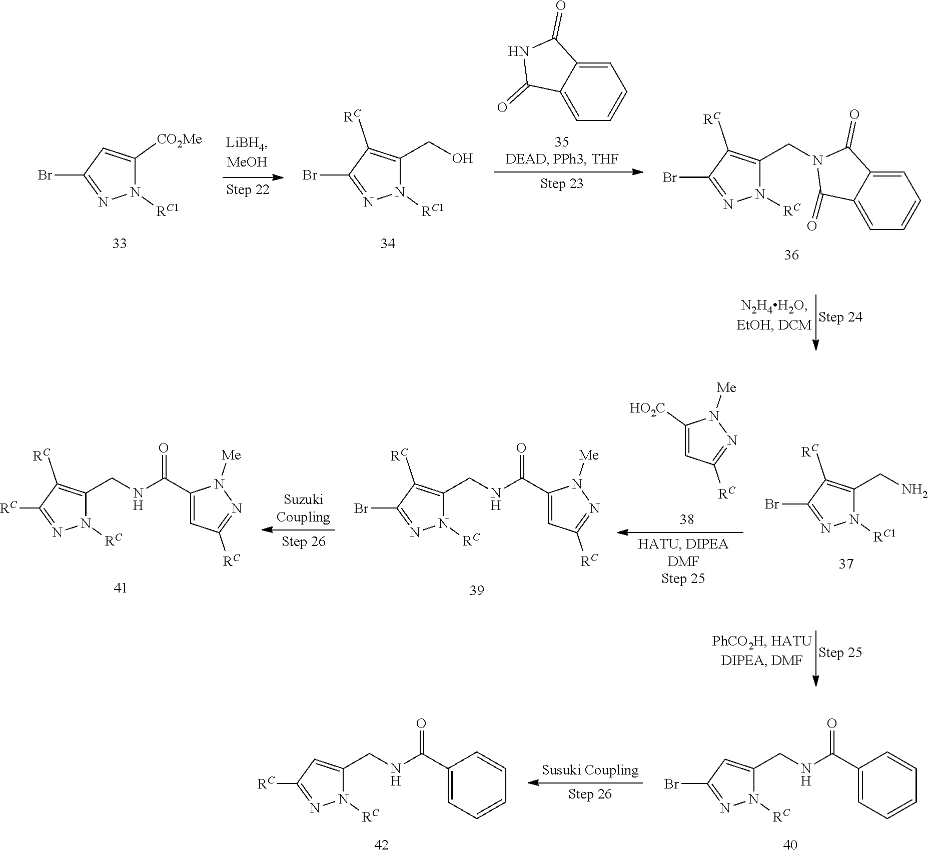

### Step 22

To a stirred solution of compound 33 (1 equiv.) in methanol (0.35 M) was added lithium borohydride (6 equiv.) portion-wise at about 0° C., and then stirring was continued at room temperature for around 1 hour. After completion of the reaction, the reaction mixture was quenched by adding ammonium chloride solution at about 0° C. and extracted with ethyl acetate. The combined organic layers were washed with water followed by brine, dried over anhydrous sodium sulfate, and concentrated under reduced pressure. The obtained crude residue was then purified by column chromatography, eluting with ethyl acetate/heptane mixtures, to afford compound 34.

### Step 23

To a stirred solution of compound 34 (1 equiv.) and phthalidimide (35) (1.1 equiv.) in THF (0.2 M) were added triphenylphosphine (1.5 equiv.) and DEAD (1.5 equiv.) at about 0° C., and then stirring was continued at room temperature for around 16 hours. After completion of the reaction, the reaction mixture was diluted with water and extracted with ethyl acetate. The combined organic layers were dried over anhydrous sodium sulfate and concentrated under reduced pressure. The obtained crude residue was then purified by column chromatography, eluting with ethyl acetate/hexane mixtures, to afford compound 36.

### Step 24

To a stirred solution of compound 36 (1 equiv.) in 1:1 DCM:EtOH (0.1 M) was added hydrazine hydrate (6 equiv.) at room temperature, and then stirring was continued at the same temperature for around 12 hours. After completion of the reaction, the reaction mixture was quenched using water and filtered through a short pad of celite. The organic layer of the filtrate was separated, dried over anhydrous sodium sulfate, and concentrated under reduced pressure. The obtained crude residue (compound 37) was used in the following reaction steps without further purification.

### Step 25

To a stirred solution of compound 37 (1 equiv.) and compound 38 or benzoic acid (1 equiv.) in DCM (0.1 M) were added HATU (1.5 equiv.) and DIPEA (3 equiv.) at about 0° C., and then stirring was continued at about 0° C. for around 2 hours. After completion of the reaction, the reaction mixture was quenched with water and extracted with DCM. The combined organic layers were dried over anhydrous sodium sulfate and concentrated under reduced pressure. The obtained crude residue was then purified by column chromatography, eluting with ethyl acetate/hexane mixtures, to afford compound 39 or compound 40.

### Step 26

To a stirred solution of compound 39 or compound 40 (1 equiv.) in 1:4 water:1,4-dioxane (0.07 M) were added cesium carbonate (2 equiv.) and boronic acid or boronic ester (2 equiv.) at room temperature. The reaction mixture was degassed under argon for around 5 minutes followed by the addition of bis(triphenylphosphine)palladium(II) dichloride (5 mol %). The reaction mixture was microwaved at about 100° C. for around 1 hour. Then the reaction mixture was allowed cool to room temperature, filtered through a pad of celite, and washed with ethyl acetate. The filtrate was washed with water, dried over anhydrous sodium sulfate, and concentrated under reduced pressure. The obtained crude residue was then purified by column chromatography, eluting with ethyl acetate, to afford compound 41 or compound 42.

Compounds 1019, 1029, 1030, 1033, and 1036-1041 were prepared from commercially available starting materials, and generally in accordance with Synthetic Scheme C and steps set forth above.

Exemplary compounds prepared in accordance with Synthetic Scheme C were characterized by NMR, HPLC, and LCMS. The data for NMR and LCMS are provided in Table 4 below.

TABLE 4

| Compound | | |
|---|---|---|
| No. | NMR | LCMS |
| 1019 | ¹H NMR (400 MHz, CDCl₃) δ 8.69 | B LCMS: 433.0 (M + H), Rt |
| | (d, J = 5.1 Hz, 1H), 8.03 (s, 1H), | 1.862 min, 99.52% |
| | 7.79 (dd, J = 1.3, 5.2 Hz, 1H), 6.82 | |
| | (s, 1H), 6.65 (s, 1H), 6.36-6.29 (m, | |
| | 1H), 4.70 (d, J = 5.9 Hz, 2H), 4.26 | |
| | (s, 3H), 3.98 (s, 3H). | |
| 1029 | 1¹H NMR (400 MHz, DMSO-d₆) δ | B LCMS: 447.1 (M + H), Rt |
| | 9.28 (br t, J = 5.5 Hz, 1H), 8.74 (d, | 2.107 min, 99.61% |
| | J = 5.1 Hz, 1H), 8.17 (s, 1H), 8.06 (d, | |
| | J = 5.0 Hz, 1H), 7.38 (s, 1H), 7.04 | |
| | (s, 1H), 4.58 (d, J = 5.6 Hz, 2H), | |
| | 4.25 (q, J = 7.2 Hz, 2H), 4.16 (s, | |
| | 3H), 1.40 (t, J = 7.2 Hz, 3H). | |
| 1030 | ¹H NMR (400 MHz, DMSO-d₆) δ | F LCMS: 446.95 (M + H), Rt |
| | 9.12 (br t, J = 5.1 Hz, 1H), 8.78 (d, | 2.003 min, 98.32% |
| | J = 4.9 Hz, 1H), 8.04 (s, 1H), 7.94 (d, | |
| | J = 4.9 Hz, 1H), 7.33 (s, 1H), 4.53 | |
| | (d, J = 5.4 Hz, 2H), 4.14 (s, 3H), | |
| | 3.95 (s, 3H), 2.31 (s, 3H). | |
| 1033 | ¹H NMR (400 MHz, DMSO-d₆) δ | FLCMS: 414.95 (M + H), Rt |
| | 9.16 (br t, J = 5.3 Hz, 1H), 8.70 (d, | 1.949 min, 99.27% |
| | J = 5.3 Hz, 1H), 8.14 (s, 1H), 8.02 (d, | |
| | J = 5.3 Hz, 1H), 7.20-6.83 (m, 3H), | |
| | 4.52 (d, J = 5.6 Hz, 2H), 4.08 (s, | |
| | 3H), 3.90 (s, 3H). | |
| 1036 | ¹H NMR (400 MHz, DMSO-d₆) δ | F LCMS: 395.0 (M + H), Rt |
| | 9.21 (t, J = 5.4 Hz, 1H), 8.14 (m, | 1.951 min, 97.24% |
| | 1H), 7.37-7.34 (m, 2H), 7.13- | |
| | 7.11 (m, 1H), 6.84 (s, 1H), 4.54 (d, | |
| | J = 5.5 Hz, 2H), 4.15 (s, 3H), 3.89 (s, | |
| | 3H), 3.85 (s, 3H). | |
| 1037 | ¹H NMR (400 MHz, DMSO-d₆) δ | F LCMS: 409.1 (M + H), Rt |
| | 9.21 (t, J = 5.4 Hz, 1H), 8.14 (d, J = | 1.984 min, 97.82% |
| | 5.4 Hz, 1H), 7.37 (s, 1H), 7.36 - | |
| | 7.34 (m, 1H), 7.12 (s, 1H), 6.84 (s, | |
| | 1H), 4.54 (d, J = 5.5 Hz, 2H), 4.33- | |
| | 4.27 (m, 2H) 4.15 (s, 3H), 3.89 (s, | |
| | 3H), 1.31 (t, J = 7 Hz, 3H). | |
| 1038 | ¹H NMR (400 MHz, DMSO-d₆) δ | F LCMS: 405.0 (M + H), Rt |
| | 9.23 (br t, J = 5.4 Hz, 1H), 8.35 (d, | 1.539 min, 99.82% |
| | J = 5.4 Hz, 1H), 7.64 (s, 1H), 7.46 | |
| | (dd, J = 1.2, 5.1 Hz, 1H), 7.38 (s, | |
| | 1H), 6.84 (s, 1H), 4.56 (d, J = 5.9 | |
| | Hz, 2H), 4.16 (s, 3H), 3.89 (s, 3H), | |
| | 2.13 (quin, J = 6.4 Hz, 1H), 0.97- | |
| | 0.88 (m, 4H). | |
| 1039 | ¹H NMR (400 MHz, DMSO-d₆) δ | F LCMS: 407.1 (M + H), Rt |
| | 8.94 (br t, J = 5.6 Hz, 1H), 8.73 (d, | 1.978 min, 99.87% |
| | J = 5.1 Hz, 1H), 8.17 (s, 1H), 8.05 (d, | |
| | J = 5.0 Hz, 1H), 7.00 (s, 1H), 6.74 | |
| | (s, 1H), 4.52 (d, J = 5.6 Hz, 2H), | |
| | 3.99 (s, 3H), 3.93 (s, 3H), 2.87 (spt, | |
| | J = 6.9 Hz, 1H), 1.19 (d, J = 6.9 Hz, | |
| | 6H). | |
| 1040 | ¹H NMR (400 MHz, DMSO-d₆) δ | I LCMS: 361.05 (M + H), Rt |
| | 9.09-9.03 (m, 1H), 8.73 (d, J = 5.0 | 1.877 min, 99.917% |
| | Hz, 1H), 8.16 (s, 1H), 8.06-8.02 | |
| | (m, 1H), 7.92-7.88 (m, 2H), 7.59- | |
| | 7.44 (m, 3H), 7.00 (s, 1H), 4.58 (d, | |
| | J = 5.5 Hz, 2H), 3.95 (s, 3H). | |
| 1041 | ¹H NMR (400 MHz, DMSO-d₆) δ | F LCMS: 396.95 (M + H), Rt |
| | 9.21 (br s, 1H), 8.45 (d, J = 2.9 Hz, | 1.695 min, 95.13% |
| | 1H), 7.74 (d, J = 6.3 Hz, 1H), 7.36 | |
| | (s, 1H), 6.74 (d, J = 3.8 Hz, 1H), | |
| | 4.58 (br d, J = 3.6 Hz, 2H), 4.15 (s, | |
| | 3H), 3.93 (s, 3H), 2.48 (br s, 3H). | |

### Example 4

### Synthesis and Characterization of Exemplary Compounds Based on Synthetic Scheme D

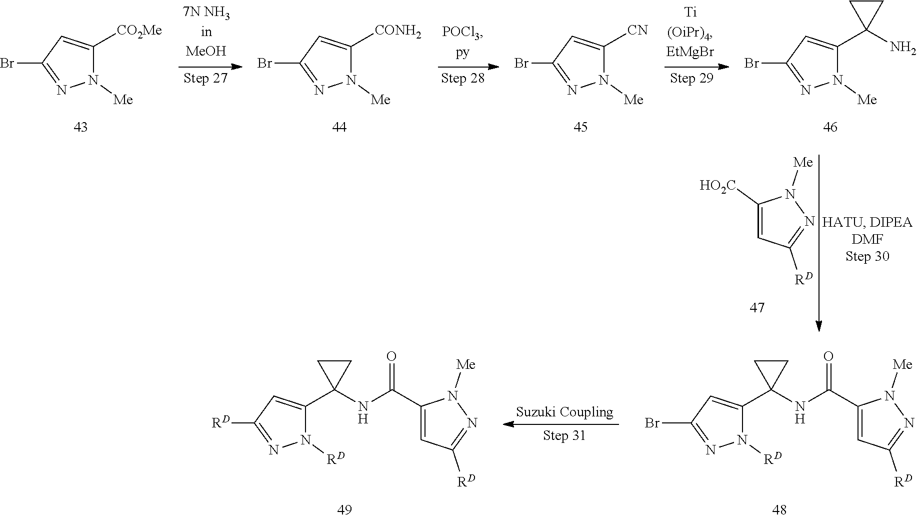

### Step 27

A suspension of compound 43 (1 equiv.) in 7 M ammonia in methanol (1 equiv.) was stirred at about 60° C. for around 24 hours. After completion of the reaction, the reaction mixture was concentrated under reduced pressure and washed with diethyl ether to afford compound 44, which was used in Step 28 below without further purification.

### Step 28

A suspension of compound 44 (1 equiv.) in phosphoryl chloride (9 equiv.) was heated at about 110° C. for around 16 hours. After completion of the reaction, the reaction was allowed to cool to room temperature, concentrated to dryness under reduced pressure, and poured onto ice. The mixture was neutralized with addition of 50% aqueous sodium hydroxide solution and extracted with ethyl acetate. The organic layer was washed with water and saturated brine solution, dried over anhydrous sodium sulfate, and concentrated under reduced pressure. The obtained crude residue was then purified by flash column chromatography, eluting with ethyl acetate/hexane mixtures, to afford compound 45.

### Step 29

To a stirred solution of compound 45 (1 equiv.) and titanium isopropoxide (2.5 equiv.) in THF (0.2 M) was added 1 M ethyl magnesium bromide (3.5 equiv.) at about −78° C. The resulting solution was stirred for around 10 minutes. The solution was then allowed to warm to room temperature and stirred for around 2 hours, followed by the addition of boron trifluoride diethyl etherate (4 equiv.). The reaction mixture was stirred at room temperature for 16 hours. After completion of the reaction, 1 N hydrochloric acid and diethyl ether were added, followed by 10% sodium hydroxide solution. The reaction mixture was extracted with ethyl acetate. The organic layer was dried over anhydrous sodium sulfate and concentrated under reduced pressure to afford compound 46.

### Step 30

To a stirred solution of compound 47 (1 equiv.) and compound 46 (2 equiv.) in DCM (0.1 M) were added HATU (1.5 equiv.) and DIPEA (3 equiv.) at about 0° C., and then stirring was continued at about 0° C. for around 2 hours. After completion of the reaction, the reaction mixture was quenched with water and extracted with DCM. The combined organic layers were dried over anhydrous sodium sulfate and concentrated under reduced pressure. The obtained crude residue was then purified by column chromatography, eluting with ethyl acetate/hexane mixtures, to afford compound 48.

### Step 31

To a stirred solution of compound 48 (1 equiv.) in 1:4 water:1,4-dioxane (0.07 M) were added cesium carbonate (2 equiv.) and boronic acid or boronic ester (2 equiv.) at room temperature. The reaction mixture was degassed under argon for around 5 minutes, followed by the addition of bis(triphenylphosphine)palladium(II) dichloride (5 mol %). The reaction mixture was degassed and then heated at about 130° C. for around 16 hours. Then the reaction mixture was allowed to cool to room temperature and filtered through a pad of celite, which was washed with ethyl acetate. The filtrate was washed with water, dried over anhydrous sodium sulfate, and concentrated under reduced pressure. The obtained crude residue was then purified by column chromatography, eluting with ethyl acetate/n-hexane, to afford compound 49.

Compounds 1034 and 1035 were prepared from commercially available starting materials, and generally in accordance with Synthetic Scheme D and steps set forth above.

Exemplary compounds prepared in accordance with Synthetic Scheme D were characterized by NMR, HPLC, and LCMS. The data for NMR and LCMS are provided in Table 5 below.

TABLE 5

| Compound | | |
|---|---|---|
| No. | NMR | LCMS |
| 1034 | ¹H NMR (400 MHz, DMSO-d₆) δ | FLCMS: 441.1 (M + H), Rt 2.073 |
| | 9.28 (br s, 1H), 8.74 (br s, 1H), | min, 99.93% |
| | 8.19 (br s, 1H), 8.03 (br s, 1H), | |
| | 7.21-7.07 (m, 2H), 7.04-6.84 | |
| | (m, 1H), 4.03 (br d, J = 3.9 Hz, | |
| | 6H), 1.29 (br s, 4H). | |
| 1035 | ¹H NMR (400 MHz, DMSO-d₆) δ | F LCMS: 459.1 (M + H), Rt 2.093 |
| | 9.34 (s, 1H), 8.74 (d, J = 4.9 Hz, | min, 98.69% |
| | 1H), 8.15 (s, 1H), 8.03 (d, J = 4.9 | |
| | Hz, 1H), 7.34 (s, 1H), 7.11 (s, 1H), | |
| | 4.05 (d, J = 11.7 Hz, 6H), 1.30 (s, | |
| | 4H). | |

### Example 5—Synthesis of 1-methyl-N-(2-(1-methyl-3-(2-(trifluoromethyl)pyridin-4-yl)-1H-pyrazol-5-yl)propan-2-yl)-3-(trifluoromethyl)-1H-pyrazole-5-carboxamide (Compound 1063)

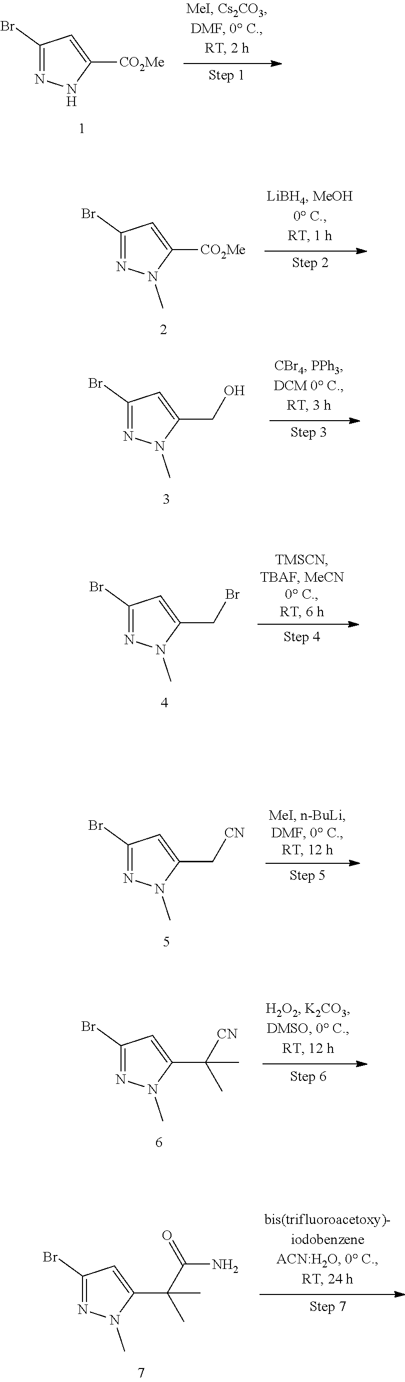

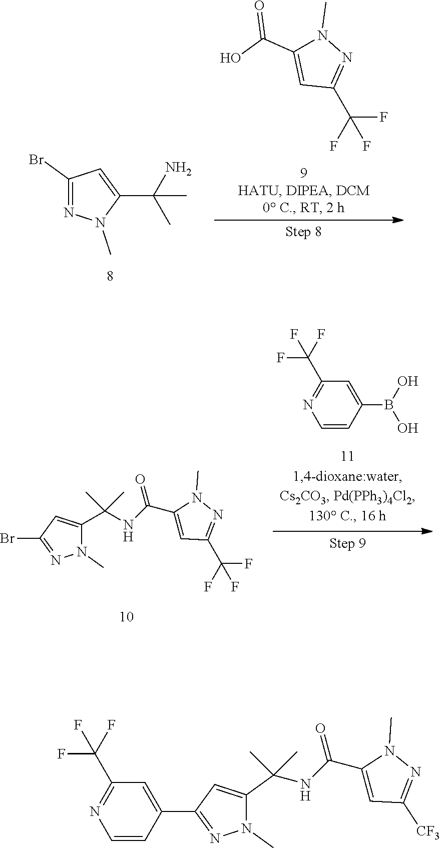 [SMILES: Cn1nc(C(F)(F)F)cc1C(=O)O ; Cn1nc(Br)cc1C(C)(C)N ; OB(O)c1ccnc(C(F)(F)F)c1 ; Cn1nc(C(F)(F)F)cc1C(=O)NC(C)(C)c1cc(Br)nn1C ; Cn1nc(C(F)(F)F)cc1C(=O)NC(C)(C)c1cc(-c2ccnc(C(F)(F)F)c2)nn1C]

### Step 1: Synthesis of methyl 3-bromo-1-methyl-1H-pyrazole-5-carboxylate (2)

To a stirred solution of compound 1 (5.0 g, 24.389 mmol) in DMF (50 mL), were added Cs₂CO₃ (15.8 g, 48.778 mmol) and methyl iodide (6.9 g, 48.778 mmol) at 0° C. The reaction mixture was stirred at room temperature for 12 hours. After completion of the reaction (monitored by TLC), the reaction mixture was diluted with water and extracted with EtOAc. The combined organic layers were washed with water followed by brine, dried over anhydrous Na₂SO₄, and concentrated under reduced pressure. The crude compound was purified by Combi-Flash chromatography (eluting with 40-50% EtOAc in heptane) to afford the title compound 2 (3.3 g, 15.031 mmol, 61% yield) as off white solid.

### Step 2: Synthesis of (3-bromo-1-methyl-1H-pyrazol-5-yl)methanol (3)

To a stirred solution of compound 2 (3.8 g, 17.348 mmol) in methanol (50 mL) was added lithium borohydride (1.89 g, 86.742 mmol) at 0° C. The reaction mixture was stirred at room temperature for 1 hour. After completion of reaction (monitored by TLC), the reaction mixture was quenched with saturated NH₄Cl solution at 0° C. and extracted with EtOAc. The combined organic layers were washed with water followed by brine, dried over anhydrous Na₂SO₄, and concentrated under reduced pressure to afford the title compound 3 (2.9 g, crude) as an off-white solid. This compound was used as such for the next step without any further purification.

### Step 3: Synthesis of 3-bromo-5-(bromomethyl)-1-methyl-1H-pyrazole (4)

To a stirred solution of compound 3 (3.0 g, 15.704 mmol) in DCM (20 mL) were added tri phenyl phosphine (6.1 g, 23.557 mmol) and CBr₄ (7.8 g, 23.557 mmol) at 0° C., and the reaction mixture was stirred at the room temperature for 3 hours. After completion of the reaction (monitored by TLC), the reaction mixture was diluted with water and extracted with EtOAc. The combined organic layers were washed with water followed by brine, dried over anhydrous Na₂SO₄, and concentrated under reduced pressure. The crude compound was purified by Combi Flash chromatography (eluting with 10-20% EtOAc in heptane) to afford the title compound 4 (2.8 g, 10.5 mmol, 66.7% yield) as off white solid.

### Step 4: Synthesis of 2-(3-bromo-1-methyl-1H-pyrazol-5-yl) acetonitrile (6)

To a stirred solution of compound 4 (2.8 g, 11.027 mmol) in ACN (25 mL) were added TBAF (1.1 mL, 16.541 mmol) and TMSCN (2.07 mL, 16.541 mmol) drop wise at 0° C. The reaction mixture was stirred at the room temperature for 6 hours. After completion of reaction (monitored by TLC), the reaction mixture was diluted with water and extracted with EtOAc. The combined organic layers were washed with water followed by brine, dried over anhydrous Na₂SO₄, and concentrated under reduced pressure. The crude compound was purified by Combi Flash chromatography (eluting with 10-20% EtOAc in heptane) to afford title compound 5 (1.6 g, 7.278 mmol, 66.0% yield) as yellow liquid.

### Step 5: Synthesis of 2-(3-bromo-1-methyl-1H-pyrazol-5-yl)-2-methylpropanenitrile (6)

To a stirred solution of compound 5 (0.1 g, 0.499 mmol) in THF (20 mL) was added nBuLi (1.6 M in hexane, 0.7 mL, 1.249 mmol) at 0° C., and the mixture was for 10 minutes. To this solution, methyl iodide (0.14 g, 0.999 mmol) was added, and the reaction mixture was stirred at room temperature for 12 hours. After completion of the reaction (monitored by TLC), the reaction mixture was diluted with ice cold water and extracted with EtOAc. The combined organic layers were washed with water followed by brine, dried over anhydrous Na₂SO₄, and concentrated under reduced pressure. The crude compound was purified by Combi-Flash chromatography (eluting with 20-30% EtOAc in heptane) to afford the title compound 6 (0.1 g, 0.346 mmol, 69.2% yield) as colorless liquid.

### Step 6: Synthesis of 2-(3-bromo-1-methyl-1H-pyrazol-5-yl)-2-methylpropanamide (7)

To a stirred solution of compound 6 (0.1 g, 0.438 mmol) in DMSO (10 mL), was added K₂CO₃ (0.06 g, 0.438 mmol) at 0° C. To this solution, H₂O₂ (0.07 mL, 2.192 mmol) was added at 0° C. temperature, and the reaction was stirred at room temperature for 12 h. After completion of the reaction (monitored by TLC), the reaction mixture was quenched with ice cold water and extracted with ethyl acetate. The combined organic layers were washed with water followed by brine, dried over anhydrous Na₂SO₄, filtered and concentrated under reduced pressure to afford title compound 7 (0.1 g, crude) as colourless liquid. This compound was used as such for the next step without any further purification.

### Step 7: Synthesis of 2-(3-bromo-1-methyl-1H-pyrazol-5-yl)propan-2-amine (8)

To a stirred solution of compound 7 (0.1 g, 0.406 mmol) in ACN:water (2:2 mL), was added bis(trifluoroacetoxy)-iodobenzene (0.174 g, 0.406 mmol) at 0° C., and the reaction was stirred at room temperature for 24 hours. After completion of the reaction (monitored by TLC), the reaction mixture was quenched with ice cold water and extracted with methyl tert-butyl ether. The aqueous layer was basified with 10% NaOH solution and extracted with EtOAc. The combined organic layers were washed with water followed by brine, dried over anhydrous Na₂SO₄, filtered and concentrated under reduced pressure to afford title compound 8 (0.07 g, crude) as pale brown solid. This compound was used as such for the next step without any further purification.

### Step 8: Synthesis of N-(2-(3-bromo-1-methyl-1H-pyrazol-5-yl)propan-2-yl)-1-methyl-3-(trifluoromethyl)-1H-pyrazole-5-carboxamide (10)

To a stirred solution of compound 9 (0.097 g, 0.504 mmol) in DCM (5 mL) were added DIPEA (0.24 mL, 1.375 mmol) and HATU (0.26 g, 0.687 mmol) at 0° C. To this solution, was compound 8 (0.1 g, 0.458 mmol) was added, and the reaction mixture was stirred at room temperature for 2 hours. After completion of the reaction (monitored by TLC), the reaction mixture was diluted with water and extracted with EtOAc. The combined organic layers were washed with water followed by brine, dried over anhydrous Na₂SO₄, and concentrated under reduced pressure. The crude compound was purified by Combi-Flash chromatography (eluting with 40-50% EtOAc in heptane) to afford the title compound 9 (0.13 g, 0.125 mmol, 27.3% yield) as off white solid.

### Step 9: Synthesis of 3-(difluoromethyl)-1-methyl-N-(2-(1-methyl-3-(2-(trifluoromethyl)pyridin-4-yl)-1H-pyrazol-5-yl)propan-2-yl)-1H-pyrazole-5-carboxamide (Compound 1063)

To a stirred solution of compound 9 (0.13 g, 0.329 mmol) in 1,4-dioxane:water (4:1 mL), were added compound 10 (0.108 g, 0.395 mmol) followed by Cs₂CO₃ (0.21 g, 0.659 mmol), and the reaction mixture was degassed with Argon gas for 15 minutes. To this solution, Pd(PPh₃)₄Cl₂ (23.1 mg, 0.033 mmol) was added under Argon atmosphere. The reaction mixture was stirred at 130° C. for 16 hours. After completion of the reaction (monitored by TLC), the reaction mixture was cooled to room temperature, filtered through a pad of Celite and washed with ethyl acetate. The filtrate was diluted with water and extracted with EtOAc. The combined organic layers were washed with water followed by brine, dried over anhydrous Na₂SO₄, filtered and concentrated under reduced pressure. The crude compound was purified by Combi Flash chromatography (eluting with 50-60% EtOAc in heptane) to afford the title compound 1063 (23 mg, 0.049 mmol, 15.0% yield) as off white solid.

Data for Compound 1063: HPLC: Rt 7.618 min, 99.14%. Column: X-Select CSH C18 (4.6*150) mm 5u Mobile Phase: A —0.1% Formic acid in water:Acetonitrile (95:05) B—Acetonitrile Flow Rate: 1.0. mL/minute Gradient program: Time (min)/B Conc.: 0.01/10, 6.0/90, 10.0/100, 12.0/100, 14/10, 18.0/10. LCMS: 461.00 (M+H), Rt 1.961 min, 99.04%. Column: X-Select CSH C₁₈ (3.0*50) mm 2.5 um Mobile Phase: A: 0.05% Formic acid in water: ACN(95:05) B: 0.05% Formic acid in ACN Inj Volume: 2.0 μL Flow Rate: 1.2. mL/minute Column oven Temp: 50° C. Gradient program: 2% B to 98% B in 2.0 minute, Hold till 3.0 min, At 3.2 min B conc is 2% up to 4.0 min. ¹H NMR (400 MHz, DMSO-d6): δ 8.79 (s, 1H), 8.75 (d, J=5.1 Hz, 1H), 8.19 (s, 1H), 8.05 (d, J=4.8 Hz, 1H), 7.52 (s, 1H), 7.09 (s, 1H), 4.03 (s, 3H), 3.90 (s, 3H), 1.74 (s, 6H).

### Example 6—Synthesis of 1-methyl-N-(1-(1-methyl-3-(2-(trifluoromethyl)pyridin-4-yl)-1H-pyrazol-5-yl)cyclobutyl)-3-(trifluoromethyl)-1H-pyrazole-5-carboxamide (Compound 1063)

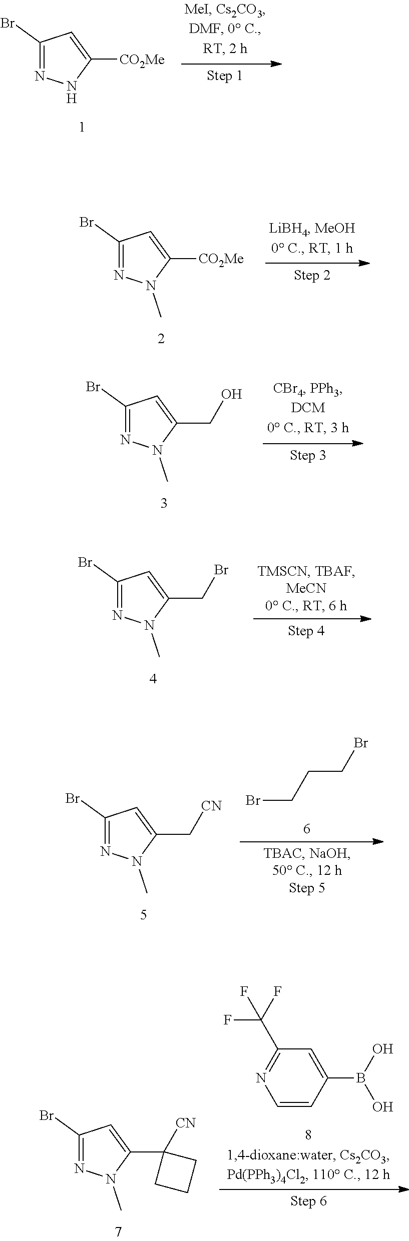

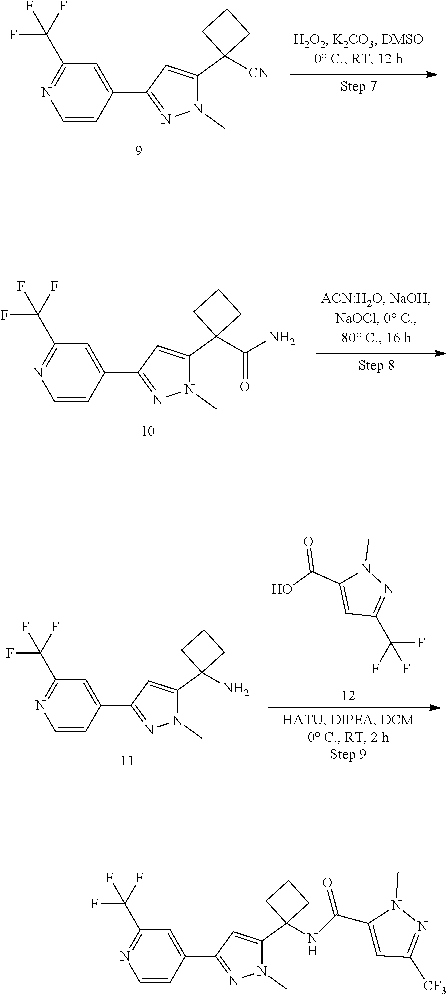 [SMILES: Cn1nc(-c2ccnc(C(F)(F)F)c2)cc1C1(C#N)CCC1 ; Cn1nc(-c2ccnc(C(F)(F)F)c2)cc1C1(C(N)=O)CCC1 ; Cn1nc(-c2ccnc(C(F)(F)F)c2)cc1C1(N)CCC1 ; Cn1nc(C(F)(F)F)cc1C(=O)O ; Cn1nc(C(F)(F)F)cc1C(=O)NC1(c2cc(-c3ccnc(C(F)(F)F)c3)nn2C)CCC1]

### Step 1: Synthesis of methyl 3-bromo-1-methyl-1H-pyrazole-5-carboxylate (2)

To a stirred solution of compound 1 (5.0 g, 24.389 mmol) in DMF (50 mL), were added Cs₂CO₃ (15.8 g, 48.778 mmol) and methyl iodide (6.9 g, 48.778 mmol) at 0° C. The reaction mixture was stirred at room temperature for 12 hours. After completion of the reaction (monitored by TLC), the reaction mixture was diluted with water and extracted with EtOAc. The combined organic layers were washed with water followed by brine, dried over anhydrous Na₂SO₄, and concentrated under reduced pressure. The crude compound was purified by Combi-Flash chromatography (eluting with 40-50% EtOAc in heptane) to afford the title compound 2 (3.3 g, 15.031 mmol, 61% yield) as off white solid.

### Step 2: Synthesis of (3-bromo-1-methyl-1H-pyrazol-5-yl)methanol (3)

To a stirred solution of compound 2 (3.8 g, 17.348 mmol) in methanol (50 mL) was added lithium borohydride (1.89 g, 86.742 mmol) at 0° C. The reaction mixture was stirred at room temperature for 1 hour. After completion of reaction (monitored by TLC), the reaction mixture was quenched with saturated NH₄Cl solution at 0° C. and extracted with EtOAc. The combined organic layers were washed with water followed by brine, dried over anhydrous Na₂SO₄, and concentrated under reduced pressure to afford the title compound 3 (2.9 g, crude) as off white solid. This compound was used as such for the next step without any further purification.

### Step 3: Synthesis of 3-bromo-5-(bromomethyl)-1-methyl-1H-pyrazole (4)

To a stirred solution of compound 3 (3.0 g, 15.704 mmol) in DCM (20 mL) were added tri phenyl phosphine (6.1 g, 23.557 mmol) and CBr₄ (7.8 g, 23.557 mmol) at 0° C., and the reaction mixture was stirred at the room temperature for 3 hours. After completion of reaction (monitored by TLC), the reaction mixture was diluted with water and extracted with EtOAc. The combined organic layers were washed with water followed by brine, dried over anhydrous Na₂SO₄, and concentrated under reduced pressure. The crude compound was purified by Combi Flash chromatography (eluting with 10-20% EtOAc in heptane) to afford the title compound 4 (2.8 g, 10.5 mmol, 66.7% yield) as off white solid.

### Step 4: Synthesis of 2-(3-bromo-1-methyl-1H-pyrazol-5-yl) acetonitrile (5)

To a stirred solution of compound 4 (2.8 g, 11.027 mmol) in ACN (25 mL) were added TBAF (1.1 mL, 16.541 mmol) and TMSCN (2.07 mL, 16.541 mmol) drop wise at 0° C. The reaction mixture was stirred at the room temperature for 6 hours. After completion of reaction (monitored by TLC), the reaction mixture was diluted with water and extracted with EtOAc. The combined organic layers were washed with water followed by brine, dried over anhydrous Na₂SO₄, and concentrated under reduced pressure. The crude compound was purified by Combi Flash chromatography (eluting with 10-20% EtOAc in heptane) to afford title compound 5 (1.6 g, 7.278 mmol, 66.0% yield) as yellow liquid.

### Step 5: Synthesis of 1-(3-bromo-1-methyl-1H-pyrazol-5-yl)cyclobutane-1-carbonitrile (7)

To a stirred mixture of compound 5 (1.2 g, 5.998 mmol), compound 6 (9.17 mL, 89.982 mmol) was added TEBAC (0.41 g, 1.799 mmol) in 50% aqueous solution of NaOH (5.9 g, 149.97 mmol). The reaction mixture was stirred at 50° C. for 12 hours. After completion of the reaction (monitored by TLC), the reaction mixture was cooled to room temperature and diluted with ice cold water and extracted with DCM. The combined organic layers were washed with water followed by brine, dried over anhydrous Na₂SO₄, and concentrated under reduced pressure. The crude compound was purified by Combi-Flash chromatography (eluting with 10-20% EtOAc in heptane) to afford the title compound 7 (1.0 g, 2.415 mmol, 40.2% yield) as colorless liquid.

### Step 6: Synthesis of 1-(1-methyl-3-(2-(trifluoromethyl)pyridin-4-yl)-1H-pyrazol-5-yl)cyclobutane-1-carbonitrile (9)

To a stirred solution of compound 7 (0.4 g, 1.666 mmol) in 1,4-dioxane:water (8:2 mL), were added compound 8 (0.54 g, 1.999 mmol) followed by Cs₂CO₃ (1.0 g, 3.331 mmol), and the reaction mixture was degassed with Argon gas for 15 min. To this solution, Pd(PPh₃)₄Cl₂ (0.11 g, 0.166 mmol) was added under Argon atmosphere. The reaction mixture was stirred at 110° C. for 12 hours. After completion of the reaction (monitored by TLC), the reaction mixture was cooled to room temperature, filtered through a pad of Celite and washed with ethyl acetate. The filtrate was diluted with water and extracted with EtOAc. The combined organic layers were washed with water followed by brine, dried over anhydrous Na₂SO₄, filtered and concentrated under reduced pressure. The crude compound was purified by Combi Flash chromatography (eluting with 30-40% EtOAc in heptane) to afford the title compound 9 (60 mg, 0.099 mmol, 23.9% yield) as off white solid.

### Step 7: Synthesis of 1-(1-methyl-3-(2-(trifluoromethyl)pyridin-4-yl)-1H-pyrazol-5-yl)cyclobutane-1-carboxamide (10)

To a stirred solution of compound 9 (0.2 g, 0.653 mmol) in DMSO (2 mL), were added K₂CO₃ (0.09 g, 0.653 mmol) and H₂O₂ (0.1 mL, 3.264 mmol) dropwise at 0° C. The reaction was stirred at room temperature for 12 hours. After completion of the reaction (monitored by TLC), the reaction mixture was quenched with ice cold water and the precipitated solid was filtered off and dried over vacuo to afford title compound 10 (0.15 g, crude) as off white solid. This compound was used as such for the next step without any further purification.

### Step 8: Synthesis of 1-(1-methyl-3-(2-(trifluoromethyl)pyridin-4-yl)-1H-pyrazol-5-yl)cyclobutan-1-amine (11)

To a stirred solution of compound 10 (0.15 g, 0.462 mmol) in 1,4 dioxane:water (4:2 mL) were added NaOH (0.04 g, 1.156 mmol) followed by NaOCI (0.85 g, 1.156 mmol) at 0° C., and the reaction mixture stirred at 80° C. for 16 hours. After completion of the reaction (monitored by TLC), the reaction mixture was cooled to room temperature, diluted with water, and extracted with DCM followed by brine. The combined organic layers were dried over anhydrous Na₂SO₄, filtered and concentrated under reduced pressure to afford the title compound 11 (0.14 g, crude) as colorless liquid. This compound was used as such for the next step without any further purification.

### Step 9: Synthesis of 1-methyl-N-(1-(1-methyl-3-(2-(trifluoromethyl)pyridin-4-yl)-1H-pyrazol-5-yl)cyclobutyl)-3-(trifluoromethyl)-1H-pyrazole-5-carboxamide (Compound 1064)

To a stirred solution of compound 12 (0.086 g, 0.445 mmol) in DCM (5 mL) were added DIPEA (0.21 mL, 1.215 mmol) and HATU (0.23 g, 0.607 mmol) at 0° C. To this solution, compound 11 (0.12 g, 0.405 mmol) was added, and the reaction mixture was stirred at room temperature for 2 hours. After completion of the reaction (monitored by TLC), the reaction mixture was diluted with water and extracted with DCM. The combined organic layers were washed with water followed by brine, dried over anhydrous Na₂SO₄, and concentrated under reduced pressure. The crude compound was purified by Combi-Flash chromatography (eluting with 40-50% EtOAc in heptane) to afford the title compound Compound 1064 (30 mg, 0.060 mmol, 14.9% yield) as off white solid.

Data: HPLC: Rt 7.704 min, 95.28%. Column: X-Select CSH C18 (4.6*150) mm 5u Mobile Phase: A —0.1% Formic acid in water:Acetonitrile (95:05) B—Acetonitrile Flow Rate: 1.0. mL/minute Gradient program: Time (min)/B Conc.: 0.01/10, 6.0/90, 10.0/100, 12.0/100, 14/10, 18.0/10. LCMS: 473.20 (M+H), Rt 1.913 min, 96.99%. Column: X-Select CSH (3.0*50) mm 2.5u Mobile Phase: A: 0.05% Formic acid in water:ACN (95:5) B: 0.05% Formic acid in ACN Inj Volume: 2.0 μL Flow Rate: 1.2. mL/minute Column oven temperature: 50 C Gradient program: 0% B to 98% B in 2.0 min, hold till 3.0 min, at 3.2 min B conc is 0% up to 4.0 min. ¹H NMR (400 MHz, CDCl₃) δ 8.71 (d, J=5.1 Hz, 1H), 8.07 (s, 1H), 7.84 (d, J=5.1 Hz, 1H), 6.84 (s, 1H), 6.79 (s, 1H), 6.40 (s, 1H), 4.15 (s, 3H), 3.94 (s, 3H), 2.83-2.71 (m, 4H), 2.26-2.15 (m, 1H), 2.09-2.00 (m, 1H).

II. Efficacy of exemplary compounds in the inhibition of KCNT1

### Example B1

### KCNT1—Patch Clamp Assay

Inhibition of KCNT1 (KNa1.1, Slack) was evaluated using a tetracycline inducible cell line (HEK-TREX). Currents were recorded using the SyncroPatch 384PE automated, patch clamp system. Pulse generation and data collection were performed with PatchController384 V1.3.0 and DataController384 V1.2.1 (Nanion Technologies). The access resistance and apparent membrane capacitance were estimated using built-in protocols. Current was recorded in perforated patch mode (10 μM escin) from a population of cells. The cells were lifted, triturated, and resuspended at 800,000 cells/ml. The cells were allowed to recover in the cell hotel prior to experimentation. Currents were recorded at room temperature. The external solution contained the following (in mM): NaCl 105, NMDG 40, KCl 4, MgCl₂ 1, CaCl₂ 5, and HEPES 10 (pH=7.4, Osmolarity ˜300 mOsm). The extracellular solution was used as the wash, reference, and compound delivery solution. The internal solution contained the following (in mM): NaCl 70, KF 70, KCl 10, EGTA 5, HEPES 5, and Escin 0.01 (pH=7.2, Osmolarity ˜295 mOsm). Escin is made at a 5 mM stock in water, aliquoted, and stored at −20° C. The compound plate was created at 2× concentrated in the extracellular solution. The compound was diluted to 1:2 when added to the recording well. The amount of DMSO in the extracellular solution was held constant at the level used for the highest tested concentration. A holding potential of −80 mV with a 100 ms step to 0 mV was used. Mean current was measured during the step to 0 mV. 100 M Bepridil was used to completely inhibit KCNT1 current to allow for offline subtraction of non-KCNT1 current. The average mean current from 3 sweeps was calculated and the percent inhibition of each compound was calculated. The percent inhibition as a function of the compound concentration was fit with a Hill equation to derive IC₅₀, slope, minimum parameters, and maximum parameters. If KCNT1 inhibition was less than 50% at the highest tested concentration or if an IC₅₀ could not be calculated, then a percent inhibition was reported in place of the IC₅₀.

Results from this example are summarized in Table 6 below. In this table, “A” indicates IC₅₀ of less than or equal to 1 μM; “B” indicates inhibition of between 1 μM to 20 μM; and “C” indicates inhibition of greater than or equal to 20 μM.

TABLE 6

| Compound No. | WT |
|---|---|
| 1001 | A |
| 1002 | A |
| 1003 | C |
| 1004 | A |
| 1005 | B |
| 1006 | B |
| 1007 | A |
| 1008 | B |
| 1009 | C |
| 1010 | B |
| 1011 | B |
| 1012 | C |
| 1013 | A |
| 1014 | B |
| 1015 | B |
| 1016 | C |
| 1017 | B |
| 1018 | B |
| 1019 | A |
| 1020 | A |
| 1021 | B |
| 1022 | C |
| 1023 | B |
| 1024 | B |
| 1025 | B |
| 1026 | C |
| 1027 | B |
| 1028 | B |
| 1029 | A |
| 1030 | A |
| 1031 | C |
| 1032 | B |
| 1033 | A |
| 1034 | A |
| 1035 | A |
| 1036 | A |
| 1037 | A |
| 1038 | A |
| 1039 | A |
| 1040 | A |
| 1041 | A |
| 1063 | A |
| 1064 | A |

### Example B2: Pharmacokinetics

Pharmacokinetic data was obtained for Compounds 1063 and 1064 as detailed herein.

Kinetic Solubility Assay: The Kinetic solubility assay employed the shake flask method followed by HPLC-UV analysis. The following step-wise procedure is used:
1) Weigh and dissolve the samples in 100% DMSO as the stock solution of 10 mM. About 10 μL (compound/Media) of stock solution is needed in this assay.
2) Add the test compounds and controls (10 mM in DMSO, 10 μL/vial) into the 50 mM pH 7.4 phosphate buffer (490 μL/well) placed in a Mini-Uniprep filter.
3) Vortex the samples of kinetic solubility for 2 minutes.
4) Incubate and shake the solubility solutions on an orbital shaker with 800 rpm at room temperature for 24 hours.
5) Centrifuge at 4000 rpm, 20° C. for 10 minutes.
6) Transfer 400 μL (with or without dilution) of each solubility supernatant into 96-deep well for analysis after the samples will be directly filtered by the syringeless filter device.
7) Determine the test compound concentration of the filtrate using HPLC-UV.
8) Inject at least 5 UV standard solutions into HPLC from low to high concentration subsequently and then test the Kinetic solubility supernatant in duplicate.
9) Use the QC samples to monitor Kinetic solubility determination process.

Log D: The Log D assay is a miniaturized 1-octanol/buffer shake flask method followed by LC/MS/MS analysis. It is typically measured by determining the partition of a compound between an organic solvent (1-octanol) and an aqueous buffer (0.1 M phosphate buffer, pH 7.4; Varied buffer pH can be set). Since log D is pH dependent, the pH of the aqueous phase is always specified and is commonly measured at pH 7.4, the physiological pH of body fluids. The following Log D method was used to calculate the Log D values in Table 8 below:
1) Dissolve appropriate test compounds in 100% DMSO to 10 mM solutions.
2) Transfer the test compounds (10 mM in DMSO; 2 μL/well) and QC samples (10 mM in DMSO; 2 L/well) from storage tubes to the 96-well polypropylene cluster tubes.
3) Add Buffer-saturated 1-octanol (149 μL/well) and 1-octanol saturated buffer (149 μL/well) to the well, respectively.
4) Vigorously mix each of the tubes on their sides for 3 minutes and then shake at a speed of 880 rpm at room temperature for 1 hour.
5) Centrifuge the tubes at 4000 rpm for 5 minutes.
6) Dilute the sample of buffer layer by a factor of 20 fold and the sample of 1-octanol layer by a factor of 200 fold with internal standard (IS) solution. Note: the dilution factor is mainly based on the properties of the compound.
7) Analyze the sample using a triple quadrupole mass spectrometer. Correct the peak areas by dilution factors and embedded internal standard, and the ratio of the corrected peak areas will be used to calculate the results (Log D value).
8) Use the QC samples to monitor the process Log D determination.
9) Data Analysis: The Log D value for each compound is calculated by the following equation:

$$Log {D}_{octbuffer} = log ( \frac{{[ 200 - fold dilution of compound ]}_{octanol} \times 200}{{[ 20 - fold compound ]}_{buffer} \times 20} ) .$$

Different dilution value in the equation will be performed with different dilution factor for sample handling.

MW, X Log P and TPSA: These data points were all calculated using Dotmatics.

Liver Microsome Metabolic Stability Assay (NADPH):
1) Test compounds were incubated at 37° C. with liver microsomes (pooled from multiple donors) at 1.0 μM in the presence of NADPH (˜1.0 mM) at 0.5 mg/ml microsomal protein.
2) Positive controls include testosterone (3A4 substrate), propafenone (2D6) and diclofenac (2C₉). They are also incubated with microsomes in the presence of NADPH.
3) Time samples (0, 5, 15, 30, 45 and 60 minutes) are removed and immediately mixed with cold acetonitrile containing internal standard (IS). Test compound incubated with microsomes without NADPH for 60 min are also included.
4) Duplicate point for each test condition (n=2).
5) Samples are analyzed by LC/MS/MS; disappearance of test compound is assessed base on peak area ratios of analyte/IS (no standard curve).
6) An excel data summary, calculated intrinsic clearance and T1/2 values are provided.
7) The following equation is used to calculate the microsome clearance:

$$\text{?} = {C}_{0} \cdot \text{?}$$

$${T}_{1 / 2} = \frac{Ln 2}{- \text{?}} = \frac{0.693}{- \text{?}}$$

$$\text{?} = \frac{0.693}{In vitro {T}_{1 / 2}} \cdot \frac{1}{mg / mL microsomal protein in reaction system}$$

$$\text{?} \text{indicates text missing or illegible when filed}$$

The mg microsomal protein/g liver weight is 45 for 5 species. The liver weight values will use 40 g/kg, 30 g/kg, 32 g/kg, 20 g/kg and 88 g/kg for rat, monkey, dog, human and mouse, respectively. The liver clearance will be calculated using CL_{int(mic)} with the following equation:

$$\text{?} = \text{?} \cdot \frac{mg microsomes}{g liver} \cdot \frac{g liver}{kg body weight} .$$

$$\text{?} \text{indicates text missing or illegible when filed}$$

Results from this example are summarized in Table 7 below.

TABLE 7

| Com- | Kinetic | | | | | |
|---|---|---|---|---|---|---|
| pound | Solubility | Log D | | | | CLᵢₙₜ |
| No. | (μM) | 7.4 | MW | XLogP | TPSA | (μL/min/mg) |
| 1063 | <1.6 | 3 | 460.38 | 3.4 | 77.6 | HClᵢₙₜ: <9.6 |
| 1064 | <1.6 | 4.4 | 472.39 | 3.5 | 77.6 | HClᵢₙₜ: <9.6 |

1. A compound of Formula (I) having a pyrazole core: 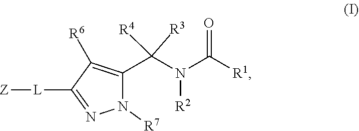
or a pharmaceutically acceptable salt thereof, wherein:
R¹ is chosen from a 5- or 6-membered heteroaryl or an aryl, wherein the heteroaryl or aryl optionally comprises at least one substituent independently chosen from an alkyl, a haloalkyl, a carbocyclyl, or —CN;
R² is —H;
R³ is chosen from —H or an alkyl;
R⁴ is chosen from —H or an alkyl,
or R³ and R⁴ are taken together with the carbon atom to which they are attached to form an optionally substituted 3- to 6-membered carbocyclyl or heterocyclyl;
Z is chosen from 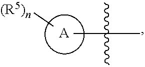
a haloalkyl, or an alkoxy;
ring A is chosen from a 5- or 6-membered heteroaryl, an aryl, a heterocyclyl or a carbocyclyl;
R⁵ is independently chosen from an alkyl, a carbocyclyl, an alkoxy, —C(O)NH₂, —CN, or a halogen, wherein the alkyl, carbocyclyl or alkoxy optionally comprises at least one halogen substituent, or wherein the alkyl optionally comprises at least one —OH substituent;
n is 0, 1, 2, 3, or 4;
L is absent or is chosen from —NR^{a}—, —CH₂—, or —O—,
R^{a} is chosen from —H or an alkyl,
R⁶ is chosen from —H or an alkyl; and
R⁷ is an alkyl.

2. The compound of claim 1, or a pharmaceutically acceptable salt thereof, wherein the compound of Formula (I) is a compound of Formula (II) having a pyrazole core: 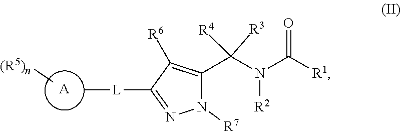
or a pharmaceutically acceptable salt thereof, wherein:
R¹ is chosen from a pyrazolyl or a phenyl, wherein the pyrazolyl or phenyl optionally comprises at least one substituent independently chosen from a C₁₋₄ alkyl, a C₁₋₄ haloalkyl, or a C₃₋₅ carbocyclyl;
R² is —H;
R³ is chosen from —H or an alkyl;
R⁴ is chosen from —H or a C₁₋₄ alkyl,
or R³ and R⁴ are taken together with the carbon atom to which they are attached to form an optionally substituted 3-5 membered carbocyclyl or heterocyclyl;
ring A is chosen from a pyridyl, a phenyl, a pyrimidinyl, a piperidinyl, or a cyclopentyl,
R⁵ is chosen from a C₁₋₄ alkyl, a C₃₋₅ carbocyclyl, a C₁₋₄ alkoxy, —C(O)NH₂, —CN, or a halogen, wherein the alkyl, carbocyclyl or alkoxy optionally comprises at least one halogen substituent, or wherein the alkyl optionally comprises at least one —OH substituent;
n is 0, 1, 2, 3, or 4;
L is absent or is chosen from —NR^{a}—, —CH₂—, or —O—,
R^{a} is chosen from —H or a C₁₋₄ alkyl,
R⁶ is chosen from —H or a C₁₋₄ alkyl; and
R⁷ is a C₁₋₄ alkyl.

3. The compound of claim 1, or a pharmaceutically acceptable salt thereof, wherein R¹ is a pyrazolyl comprising at least one substituent chosen from —CH₃, —CF₃, —C(CH₃)₃, —CHF₂, —CH(CH₃)₂, or a cyclopropyl.

4. The compound of claim 1, or a pharmaceutically acceptable salt thereof, wherein R¹ is a phenyl.

5. The compound of claim 1, or a pharmaceutically acceptable salt thereof, wherein R⁴ is chosen from —H or —CH₃.

6. The compound of claim 1, or a pharmaceutically acceptable salt thereof, wherein R³ and R⁴ are taken together with the carbon atom to which they are attached to form an optionally substituted cyclopropyl, cyclobutyl, or oxetanyl.

7. The compound of claim 1, or a pharmaceutically acceptable salt thereof, wherein ring A is a pyridyl.

8. The compound of claim 1, or a pharmaceutically acceptable salt thereof, wherein R⁵ at each occurrence is independently chosen from —CH₃, —CH₂CH₃, —CF₃, —OCH₃, —OCH₂CH₃, —OCH(CH₃)₂, —CH₂OH, —CN, —C(O)NH₂, or a cyclopropyl.

9. The compound of claim 1, or a pharmaceutically acceptable salt thereof, wherein n is 0, 1, or 2.

10. The compound of claim 1, or a pharmaceutically acceptable salt thereof, wherein L is absent.

11. The compound of claim 1, or a pharmaceutically acceptable salt thereof, wherein R⁶ is chosen from —H or —CH₃.

12. The compound of claim 1, or a pharmaceutically acceptable salt thereof, wherein R⁷ is chosen from —CH₃ or —CH₂CH₃.

13. The compound of claim 1, or a pharmaceutically acceptable salt thereof, wherein the compound of Formula (I) is chosen from a compound of Formula (II-A), (II-B), or (II-C) having a pyrazole core: 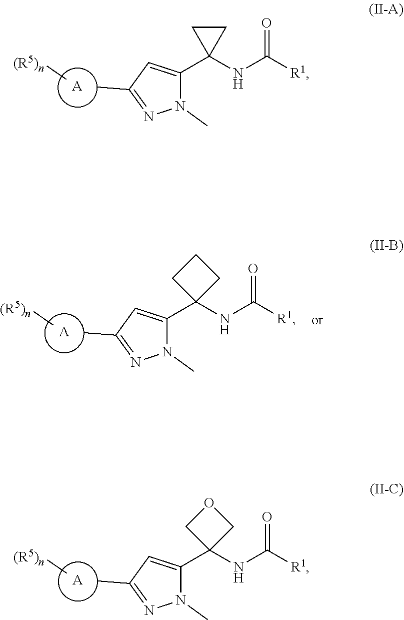
or a pharmaceutically acceptable salt thereof.

14. The compound of claim 13, or a pharmaceutically acceptable salt thereof, wherein the compound of Formula (I) is chosen from a compound of Formula (III-A), (III-B), or (III-C) having a pyrazole core: 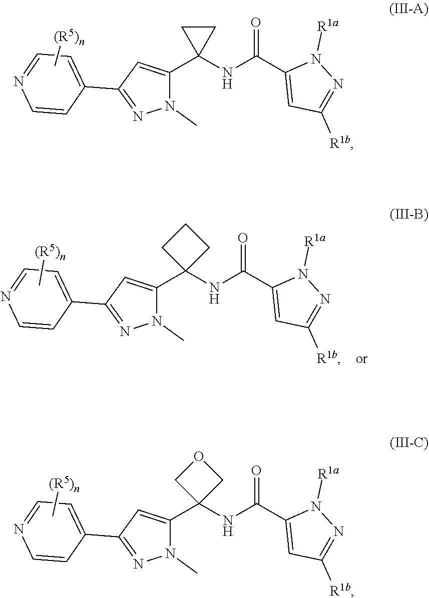
or a pharmaceutically acceptable salt thereof, wherein:
R^{1a} is chosen from —CH₃, —C(CH₃)₃, —CHF₂, —CH(CH₃)₂, or a cyclopropyl;
R^{1b} is chosen from —CH₃, —CF₃, —C(CH₃)₃, —CHF₂, —CH(CH₃)₂, or a cyclopropyl; and
R⁵ at each occurrence is independently chosen from a C₁₋₄ alkyl, a C₁₋₄ haloalkyl, a C₁₋₄ alkoxy, or a C₃₋₅ carbocyclyl.

15. The compound of claim 14, or a pharmaceutically acceptable salt thereof, wherein the compound of Formula (I) is chosen from a compound of Formula (III-A-i), (III-B-i), or (III-C-i) having a pyrazole core: 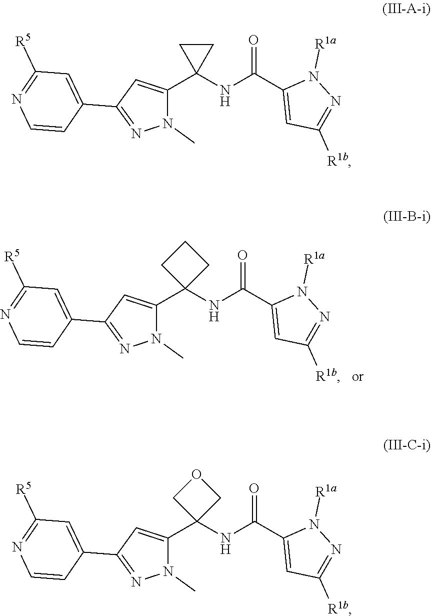
or a pharmaceutically acceptable salt thereof, wherein:
R^{1a} is chosen from —CH₃, —C(CH₃)₃, —CHF₂, —CH(CH₃)₂, or a cyclopropyl;
R^{1b} is chosen from —CH₃, —CF₃, —C(CH₃)₃, —CHF₂, —CH(CH₃)₂, or a cyclopropyl; and
R⁵ is chosen from —CF₃, —CH₃, —CH₂CH₃, —OCH₃, —OCH₂CH₃, or a cyclopropyl.

16. The compound of claim 14, or a pharmaceutically acceptable salt thereof, wherein the compound of Formula (I) is chosen from a compound of Formula (III-D-i) or (III-E-i) having a pyrazole core: 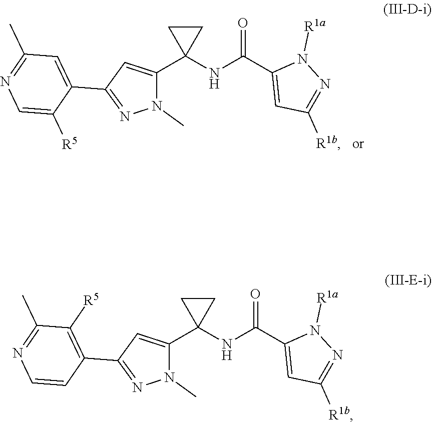
or a pharmaceutically acceptable salt thereof, wherein:
R^{1a} is chosen from —CH₃, —C(CH₃)₃, —CHF₂, —CH(CH₃)₂, or a cyclopropyl;
R^{1b} is chosen from —CH₃, —CF₃, —C(CH₃)₃, —CHF₂, —CH(CH₃)₂, or a cyclopropyl; and
R⁵ is chosen from —F or —CN.

17. The compound of claim 1, wherein the compound is chosen from any of the following compounds or enantiomers thereof, or a pharmaceutically acceptable salt thereof:

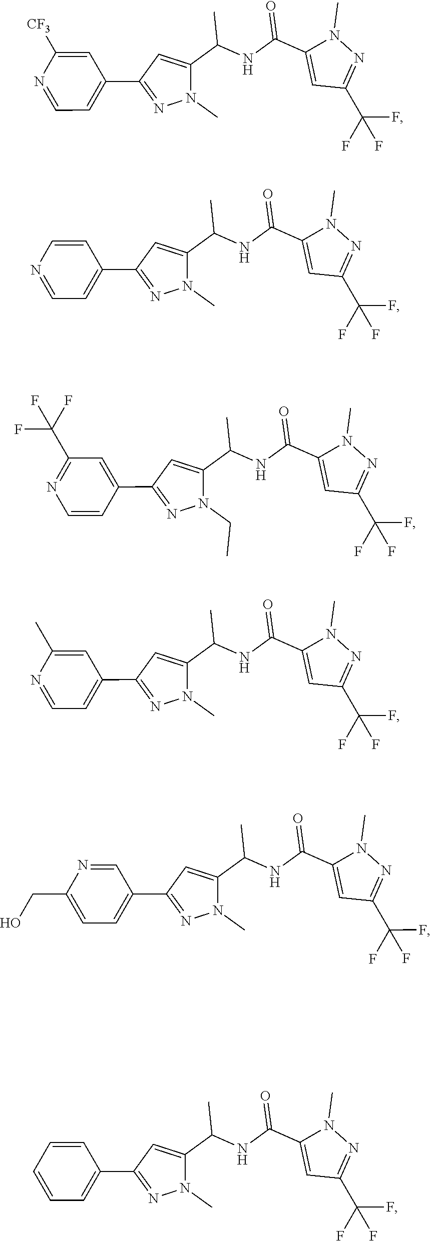

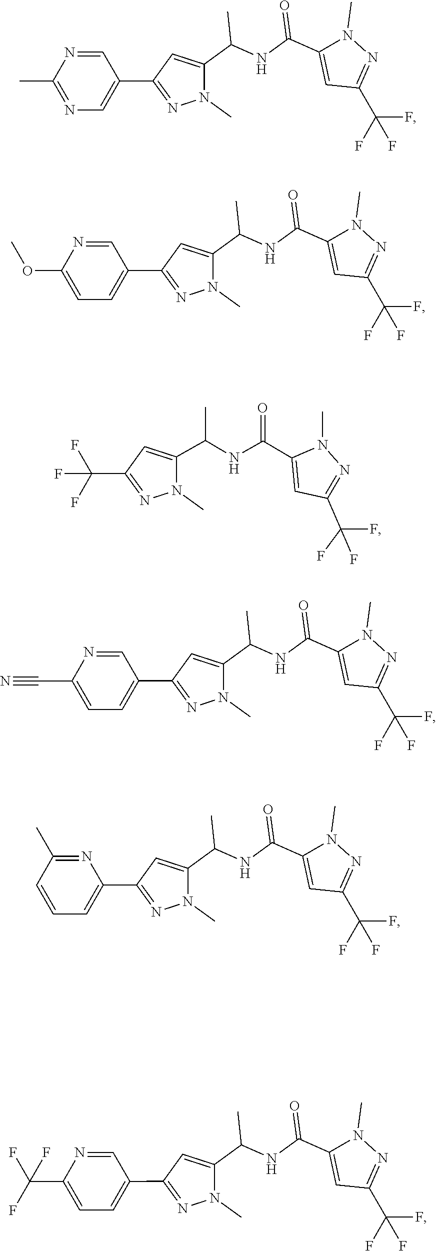

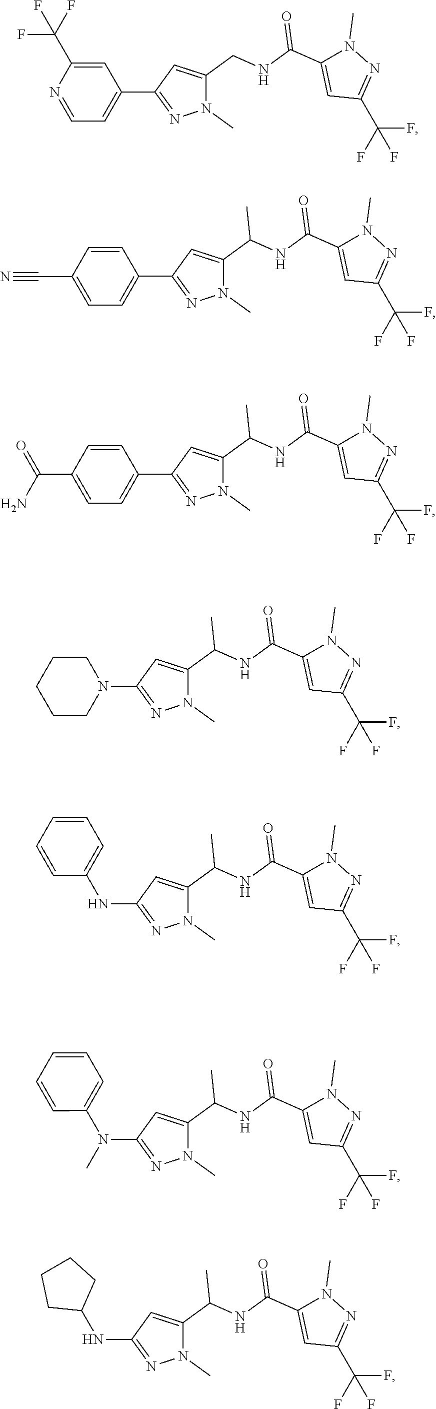

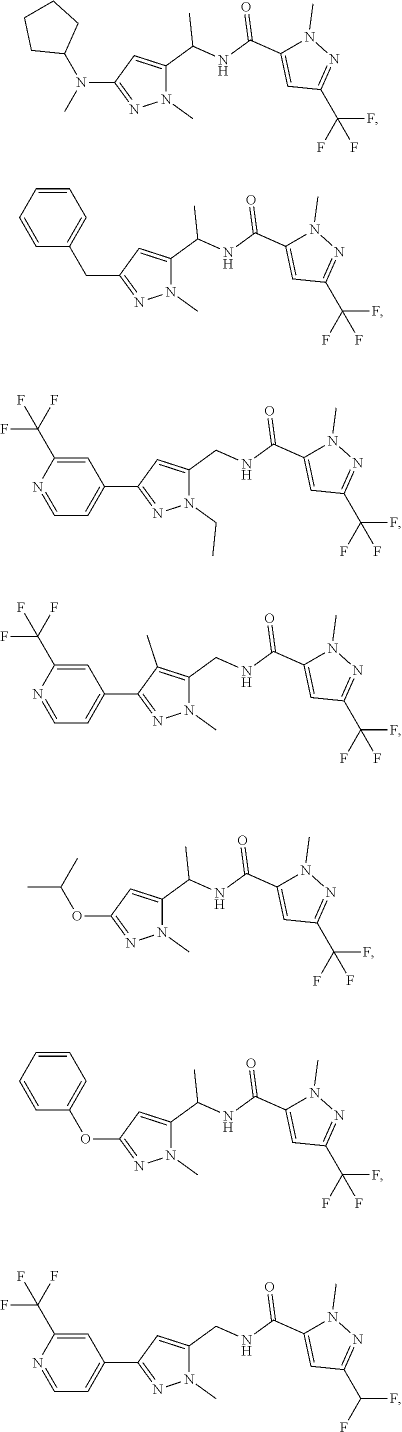

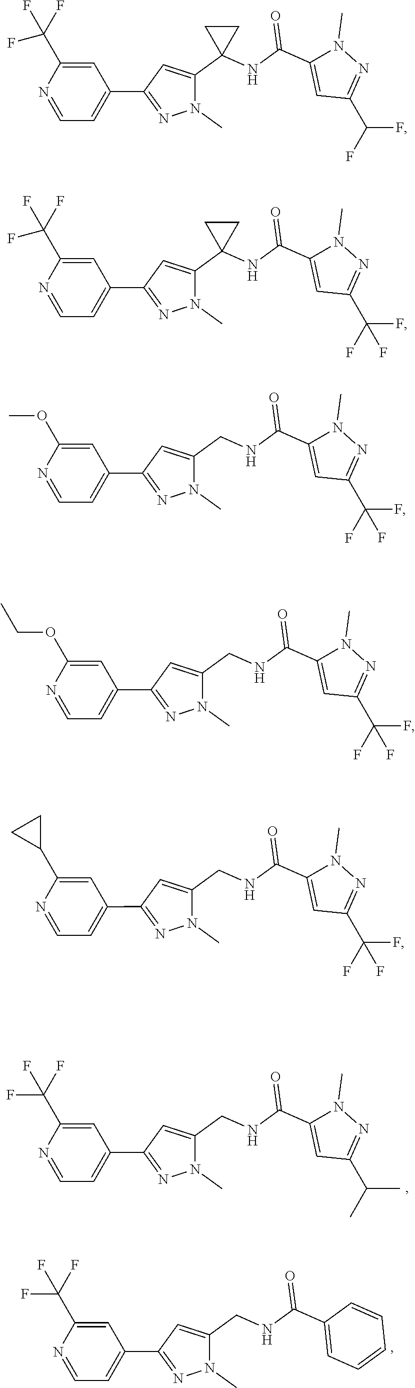

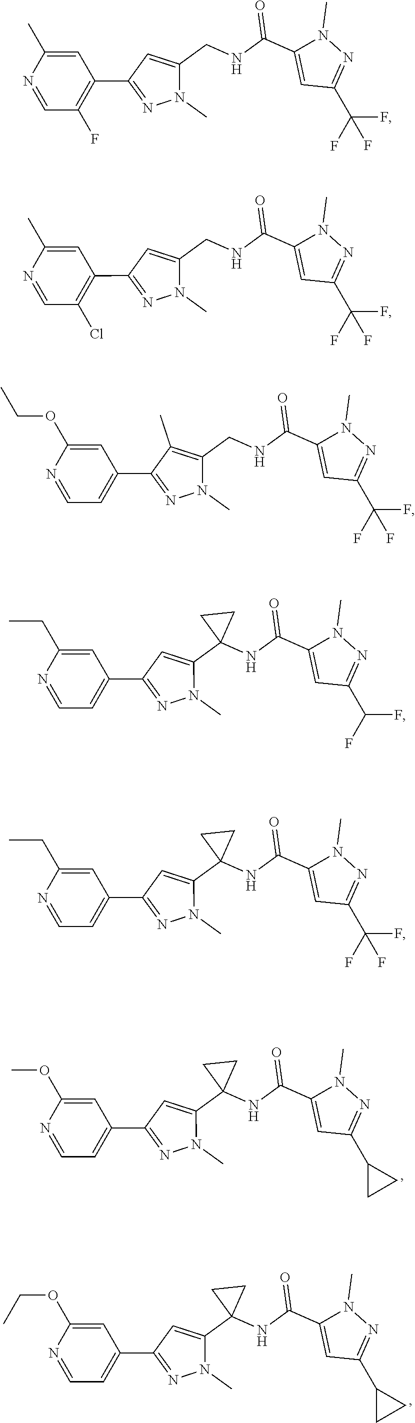

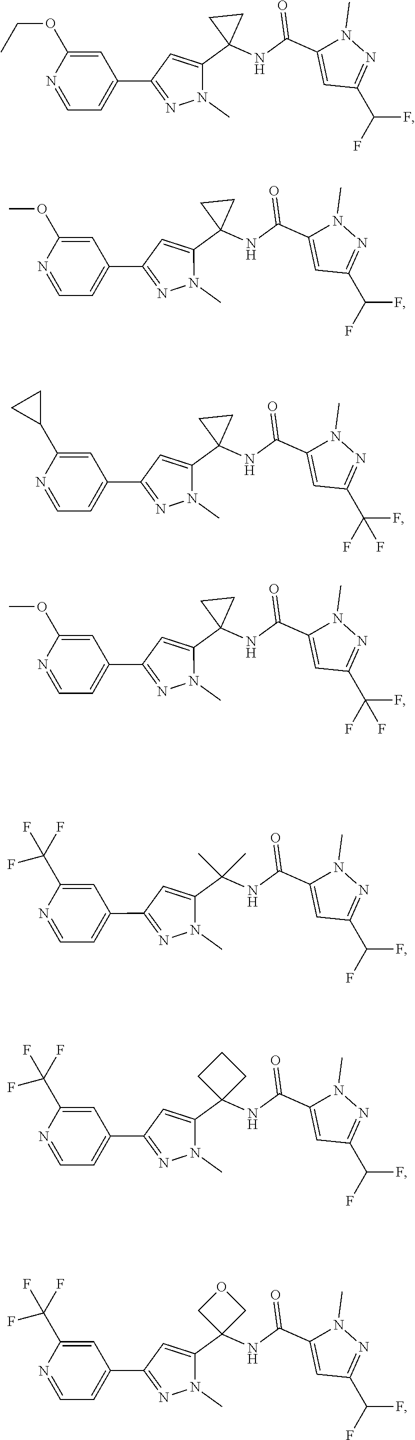

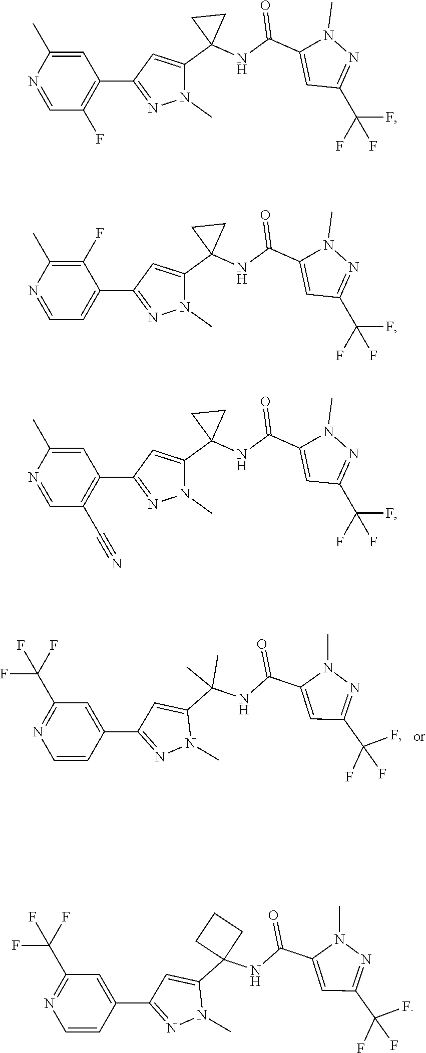

18. A pharmaceutical composition, comprising:
a compound of claim 1, or a pharmaceutically acceptable salt thereof; and
at least one pharmaceutically acceptable excipient.

19. A method of treating a neurological disorder, a disorder associated with excessive neuronal excitability, or a disorder associated with a gain-of-function mutation of a gene, wherein the method comprises administering to a subject in need thereof an effective amount of a compound of claim 1.

20. The method of claim 19, wherein the disorder is a disorder associated with a gain-of-function mutation of KCNT1.

21. The method of claim 19, wherein the neurological disorder, the disorder associated with excessive neuronal excitability, or the disorder associated with a gain-of-function mutation of a gene is epilepsy, an epilepsy syndrome, an encephalopathy, a genetic or pediatric epilepsy, a genetic or pediatric epilepsy syndrome, a cardiac dysfunction, malignant migrating focal seizures of infancy (MMFSI) or epilepsy of infancy with migrating focal seizures (EIMFS), autosomal dominant nocturnal frontal lobe epilepsy (ADNFLE), West syndrome, infantile spasms, epileptic encephalopathy, focal epilepsy, Ohtahara syndrome, developmental and epileptic encephalopathy, Lennox-Gastaut syndrome, seizures, leukodystrophy, leukoencephalopathy, intellectual disability, multifocal epilepsy, drug-resistant epilepsy, temporal lobe epilepsy, cerebellar ataxia, cardiac arrhythmia, Brugada syndrome, and myocardial infarction, pain and related conditions, a muscle disorder, itch and pruritis, ataxia, a psychiatric disorder, a learning disorder, Fragile X, neuronal plasticity, an autism spectrum disorder, epileptic encephalopathy with SCN1A, SCN2A, and/or SCN8A mutations, early infantile epileptic encephalopathy, Dravet syndrome, Dravet syndrome with SCN1A mutation, generalized epilepsy with febrile seizures, intractable childhood epilepsy with generalized tonic-clonic seizures, benign familial neonatal-infantile seizures, SCN2A epileptic encephalopathy, focal epilepsy with SCN3A mutation, cryptogenic pediatric partial epilepsy with SCN3A mutation, SCN8A epileptic encephalopathy, Rasmussen encephalitis, malignant migrating partial seizures of infancy, KCNQ2 epileptic encephalopathy, or KCNT1 epileptic encephalopathy.

22.-31. (canceled)
